(19) United States
(12) Patent Application Publication
(10) Pub. No.: US 2021/0101990 A1
(43) Pub. Date: Apr. 8, 2021

(54) COMPOSITIONS AND METHODS FOR THE DEPLETION OF CD134+ CELLS

(71) Applicant: Magenta Therapeutics, Inc., Cambridge, MA (US)

(72) Inventors: Megan D. Hoban, Medford, MA (US); Anthony Boitano, Newton, MA (US); Michael Cooke, Boston, MA (US)

(73) Assignee: Magenta Therapeutics, Inc., Cambridge, MA (US)

(21) Appl. No.: 16/932,319

(22) Filed: Jul. 17, 2020

Related U.S. Application Data
(63) Continuation of application No. PCT/IB2019/050446, filed on Jan. 18, 2019.
(60) Provisional application No. 62/619,106, filed on Jan. 18, 2018.

(51) Int. Cl. C07K 16/28; A61K 47/68

(52) U.S. Cl. CPC C07K 16/2878; A61K 35/28; A61K 47/6849; A61K 47/6831

(57) ABSTRACT

The invention provides methods of preventing and treating graft-versus-host-disease and autoimmune diseases, such as those arising from transplant therapy, by selective depletion of hematopoietic cells through the use of antibody-drug conjugates (ADCs) that specifically bind CD134 or CD278. The compositions and methods described herein can be used to treat a variety of pathologies, including autoimmune diseases, stem cell disorders, and other blood conditions.

### RELATED APPLICATIONS

This application is a continuation of PCT Appln. No. PCT/IB2019/050446, filed on Jan.18, 2019, which claims priority to U.S. Provisional Application No. 62/619,106, filed on Jan. 18, 2018. The contents of the aforementioned applications are incorporated by reference herein in their entirety.

### SEQUENCE LISTING

The Sequence Listing submitted herewith as a text file named “M103034_1380US_Seq_List,” created on Jul. 17, 2020, and having a size of 4 kilobytes is hereby incorporated by reference pursuant to 37 C.F.R. §1.52(e)(5).

### FIELD OF THE INVENTION

The present invention relates to the field of transplant therapy and provides methods for the treatment of autoimmune diseases or graft-versus-host disease (GVHD) by administration of antibodies, antibody-drug conjugates, and ligand-drug conjugates capable of binding an antigen expressed by hematopoietic cells.

### BACKGROUND OF THE INVENTION

While hematopoietic stem cells have significant therapeutic potential, a limitation that has hindered their use in the clinic has been the development of graft-versus-host disease (GVHD) some days or weeks after the cell transplant. While significant advances have been made with regard to the treatment of GVHD following transplantation, there is still a need in the art for improved methods, particularly with respect to reducing mortality rates from GVHD. Conventional treatment of GVHD requires systemic immunosuppressive therapy with potent drugs such as corticosteroids and cyclosporine. Agents such as mycophenolate mofetil, rapamycin (sirolimus), imatinib, and rituximab are used in patients with steroid-refractory GVHD. However, these treatments have limited efficacy and often cause severe adverse effects. Only 50% of patients with GVHD are able to discontinue immunosuppressive treatment within 5 years after diagnosis, and 10% require continued treatment beyond 5 years. The remaining 40% die or develop recurrent malignancy before GVHD resolves. Five year survival rates of patients with high risk GVHD (platelet counts <100,000/microliter or progressive onset from GVHD) is only 40-50%. Thus, the development of innovative strategies to prevent and treat GVHD represents an important unmet clinical need.

Like, GVHD, autoimmune diseases such as multiple sclerosis, rheumatoid arthritis, intestinal bowel disease, psoriasis, lupus, and Type 1 diabetes are characterized by an abnormal immune response directed against normal self tissues. Autoimmune diseases are characterized by production of autoreactive T cells and antibodies reactive with host tissues (autoantibodies). Traditional therapies for autoimmune disease include immunosuppressive agents that globally dampen immune responses. The benefits of such agents are often tempered by susceptibility to opportunistic infections, long-term risk of malignancy, toxicity and other unfavorable side effects. Thus, there is a need to develop a strategy to more specifically target the cellular mediators of both GVHD and autoimmune diseases.

### SUMMARY OF THE INVENTION

The present invention provides methods for preventing and treating acute and chronic forms of graft-versus-host disease (GVHD) or autoimmune diseases in a patient such as a human patient, receiving hematopoietic stem cell transplant therapy so as to reduce the morbidity and mortality associated with GVHD and autoimmune diseases. The invention additionally features methods of treating a variety of hematopoietic conditions, such as sickle cell anemia, thalassemia, Fanconi anemia, Wiskott-Aldrich syndrome, adenosine deaminase deficiency-severe combined immunodeficiency, metachromatic leukodystrophy, Diamond-Blackfan anemia and Schwachman-Diamond syndrome, human immunodeficiency virus infection, and acquired immune deficiency syndrome, among others.

In certain embodiments, the methods and compositions disclosed herein are used to treat or prevent allograft rejection in a human patient who received (or is going to be receiving) an allogeneic bone marrow transplant.

The invention features methods of treating a patient with antibodies, antibody-drug conjugates (ADCs), ligands, and ligand-drug conjugates capable of binding proteins expressed by hematopoietic cells, such as CD134 or CD278, so as to deplete a population of hematopoietic cells, such as T cells, within the patient. This selective depletion of T cells in turn improves overall and relapse-free patient survival while significantly decreasing GVHD and autoimmune diseases.

In a first aspect, the invention features a method of treating or preventing graft-versus-host disease (GVHD) in a human patient in need thereof by administering to the patient an effective amount of an antibody, or antigen-binding fragment thereof, or an antibody drug conjugate (ADC), capable of binding CD134.

In a second aspect, the invention provides a method of depleting a population of CD134 positive cells in a human patient suffering from or at risk for GVHD by administering to the patient an effective amount of an antibody, or antigen-binding fragment thereof, or an antibody drug conjugate (ADC),capable of binding CD134.

In a third aspect, the invention features a method of treating an autoimmune disease in a human patient in need thereof by administering to the patient an effective amount of an antibody, or antigen-binding fragment thereof, or antibody drug conjugate (ADC), capable of binding CD134.

In a fourth aspect, the invention provides a method of depleting a population of CD134 positive cells in a human patient suffering from or at risk for an autoimmune disease by administering to the patient an effective amount of an antibody, or antigen-binding fragment thereof, or an antibody drug conjugate (ADC), capable of binding CD134.

In another aspect, the invention features a method of treating or preventing allograft rejection in a human patient in need thereof by administering to the patient an effective amount of an antibody, or antigen-binding fragment thereof, or antibody drug conjugate (ADC), capable of binding CD134. In certain embodiments, the allograft rejection is host versus graft disease (HvGD).

In another aspect, the invention features a method of depleting a population of CD134 positive cells in a human patient suffering from or at risk for an allograft rejection by administering to the patient an effective amount of an antibody, or antigen-binding fragment thereof, or antibody drug conjugate (ADC), capable of binding CD134. In certain embodiments, the allograft rejection is host versus graft disease (HvGD).

In another aspect, the invention features a method of treating or preventing GVHD in a human patient in need thereof by administering to the patient an effective amount of an antibody, or antigen-binding fragment thereof, or an antibody drug conjugate (ADC), capable of binding CD278.

In a another aspect, the invention provides a method of depleting a population of CD278 positive cells in a human patient suffering from or at risk for GVHD by administering to the patient an effective amount of an antibody, or antigen-binding fragment thereof, or an antibody drug conjugate (ADC),capable of binding CD278.

In a another aspect, the invention features a method of treating an autoimmune disease in a human patient in need thereof by administering to the patient an effective amount of an antibody, or antigen-binding fragment thereof, or antibody drug conjugate (ADC), capable of binding CD278.

In a another aspect, the invention provides a method of depleting a population of CD278+ cells in a human patient suffering from or at risk for an autoimmune disease by administering to the patient an effective amount of an antibody, or antigen-binding fragment thereof, or an antibody drug conjugate (ADC), capable of binding CD278.

In another aspect, the invention features a method of treating or preventing allograft rejection in a human patient in need thereof by administering to the patient an effective amount of an antibody, or antigen-binding fragment thereof, or antibody drug conjugate (ADC), capable of binding CD278. In certain embodiments, the allograft rejection is host versus graft disease (HvGD).

In another aspect, the invention features a method of depleting a population of CD278 positive cells in a human patient suffering from or at risk for an allograft rejection by administering to the patient an effective amount of an antibody, or antigen-binding fragment thereof, or antibody drug conjugate (ADC), capable of binding CD278. In certain embodiments, the allograft rejection is host versus graft disease (HvGD).

In some embodiments, the antibody, antigen-binding fragment thereof, or antibody-drug conjugate binds human CD134, the amino acid sequence of which is provided below (NCBI Reference Sequence: NP_003318.1):

MCVGARRLGR GPCAALLLLG LGLSTVTGLH CVGDTYPSND
RCCHECRPGN GMVSRCSRSQNTVCRPCGPG FYNDVVSSKP
CKPCTWCNLR SGSERKQLCT ATQDTVCRCR
AGTQPLDSYKPGVDCAPCPP GHFSPGDNQA CKPWTNCTLA
GKHTLQPASN SSDAICEDRD PPATQPQETQGPPARPITVQ
PTEAWPRTSQ GPSTRPVEVP GGRAVAAILG LGLVLGLLGP
LAILLALYLLRRDQRLPPDA HKPPGGGSFR TPIQEEQADA
HSTLAKI: (SEQ ID NO: 1)

In some embodiments, the antibody, antigen-binding fragment thereof, or antibody-drug conjugate binds human CD278, the amino acid sequence of which is provided below (NCBI Reference Sequence: NP_036224.1):

(SEQ ID NO: 2)
MKSGLWYFFLFCLRIKVLTGEINGSANYEMFIFHNGGVQILCKYPDIVQQ
FKMQLLKGGQILCDLTKTKGSGNTVSIKSLKFCHSQLSNNSVSFFLYNLD
HSHANYYFCNLSIFDPPPFKVTLTGGYLHIYESQLCCQLKFWLPIGCAAF
VVVCILGCILICWLTKKKYSSSVHDPNGEYMFMRAVNTAKKSRLTDVTL

In some embodiments, the anti-CD134 or anti-CD278 antibody, or antigen-binding fragment thereof is selected from the group consisting of a monoclonal antibody or antigen-binding fragment thereof, a polyclonal antibody or antigen-binding fragment thereof, a humanized antibody or antigen-binding fragment thereof, a bispecific antibody or antigen-binding fragment thereof, a dual-variable immunoglobulin domain, a single-chain Fv molecule (scFv), a diabody, a triabody, a nanobody, an antibody-like protein scaffold, a Fv fragment, a Fab fragment, a F(ab′)₂ molecule, and a tandem di-scFv. In another embodiment, the anti-CD134 or anti-CD278 antibody, or antigen-binding fragment thereof is an IgG and contains a human IgG1, IgG2, IgG3, or IgG4 isotype Fc domain.

In some embodiments, the anti-CD134 or anti-CD278 antibody has an isotype selected from the group consisting of IgG, IgA, IgM, IgD, and IgE.

In some embodiments, the Fc domain is a human IgG1 isotype Fc domain. In some embodiments, the Fc domain is a human IgG2 isotype Fc domain. In some embodiments, the Fc domain is a human IgG3 isotype Fc domain. In some embodiments, the Fc domain is a human IgG4 isotype Fc domain.

In another aspect, the invention features a method of treating GVHD in a human patient in need thereof, the method comprising administering to the patient an effective amount of an anti-CD134 ADC.

In another aspect, the invention features a method of depleting a population of CD134 positive cells in a human patient suffering from or at risk for GVHD, the method comprising administering to the patient an effective amount of an anti-CD134 or a soluble CD134 ligand.

In another aspect, the invention features a method of treating an autoimmune disease in a human patient in need thereof, the method comprising administering to the patient an effective amount of an anti-CD134 ADC or a soluble CD134 ligand.

In another aspect, the invention features a method of depleting a population of CD134 positive cells in a human patient suffering from or at risk for an autoimmune disease, the method comprising administering to the patient an effective amount of an anti-CD134 ADC or a soluble CD134 ligand.

In another aspect, the invention features a method of treating GVHD in a human patient in need thereof, the method comprising administering to the patient an effective amount of an anti-CD278 ADC.

In another aspect, the invention features a method of depleting a population of CD134 positive cells in a human patient suffering from or at risk for GVHD, the method comprising administering to the patient an effective amount of an anti-CD278 ADC.

In another aspect, the invention features a method of treating an autoimmune disease in a human patient in need thereof, the method comprising administering to the patient an effective amount of an anti-CD278 ADC.

In another aspect, the invention features a method of depleting a population of CD278 positive cells in a human patient suffering from or at risk for an autoimmune disease, the method comprising administering to the patient an effective amount of an anti-CD278 ADC.

In another aspect, the invention features a method of depleting alloreactive T cells in a human patient who received an allogeneic transplant, the method comprising administering an anti-CD134 ADC (or an anti-CD278 ADC) to the human patient such that alloreactive T cells are depleted, wherein the ADC comprises an anti-CD134 antibody (or an anti-CD278) conjugated to a cytotoxin. In some embodiments, the transplant is a bone marrow transplant, a peripheral blood transplant, or a cord blood transplant. In some embodiments, the transplant comprises hematopoietic cells. In some embodiments, the hematopoietic stem cells or progeny thereof maintain hematopoietic stem cell functional potential after two or more days following transplantation of the hematopoietic stem cells into the patient. In some embodiments, the cytotoxin is an RNA polymerase inhibitor. In other embodiments, the RNA polymerase inhibitor is an amatoxin.

In some embodiments, the antibody (e.g., anti-CD134 antibody or anti-CD278 antibody) or antigen-binding fragment thereof is conjugated to a cytotoxin, such as a microtubule-binding agent or an RNA polymerase inhibitor. In some embodiments, the anti-CD134 or anti-CD278 antibody or antigen-binding fragment thereof, or soluble CD134 or CD278 ligand is conjugated to a microtubule-binding agent by way of a linker. In some embodiments, the anti-CD134 or anti-CD278 antibody or antigen-binding fragment thereof, or soluble CD134 or CD278 ligand is conjugated to an RNA polymerase inhibitor by way of a linker.

In some embodiments, the microtubule-binding agent is a maytansine or a maytansinoid.

In some embodiments, the maytansinoid is selected from the group consisting of DM1, DM3, and DM4, and maytansinol.

In some embodiments, the maytansinoid is a maytansinol analog.

In some embodiments, the RNA polymerase inhibitor is an amatoxin.

In some embodiments of any of the above aspects, the cytotoxin is an amatoxin or derivative thereof, such as α-amanitin, β-amanitin, γ-amanitin, ε-amanitin, amanin, amaninamide, amanullin, amanullinic acid, or proamanullin. In one embodiment, the cytotoxin is an amanitin. In some embodiments of any of the above aspects, the cytotoxin is an amatoxin, and the antibody or the antigen-binding fragment thereof is conjugated to the amatoxin through a linker and chemical moiety to form an ADC represented by the formula Ab-Z-L-Am, wherein Ab is the antibody or antigen-binding fragment thereof, L is a linker, Z is a chemical moiety, and Am is the amatoxin.

In some embodiments, the amatoxin is conjugated to a linker. In some embodiments, the amatoxin-linker conjugate Am-L-Z is represented by formula (I)

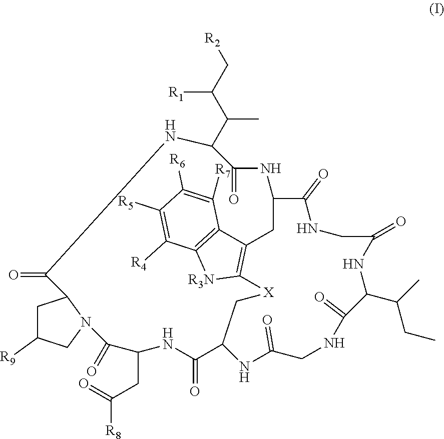

wherein R₁ is H, OH, OR_{A}, or OR_{C};

R₂ is H, OH, OR_{B}, or OR_{C};

R_{A} and R_{B}, when present, together with the oxygen atoms to which they are bound, combine to form an optionally substituted 5-membered heterocycloalkyl group;

R₃ is H, R_{C}, or R_{D};

R₄ is H, OH, OR_{C}, OR_{D}, R_{C}, or R_{D};

R₅ is H, OH, OR_{C}, OR_{D}, R_{C}, or R_{D};

R₆ is H, OH, OR_{C}, OR_{D}, R_{C}, or R_{D};

R₇ is H, OH, OR_{C}, OR_{D}, R_{C}, or R_{D};

R₈ is OH, NH₂, OR_{C}, OR_{D}, NHR_{C}, or NR_{C}R_{D};

R₉ is H, OH, OR_{C}, or OR_{D};

X is —S—, —S(O)—, or —SO₂—;

R_{C} is -L-Z;

R_{D} is optionally substituted alkyl (e.g., C₁-C₆ alkyl), optionally substituted heteroalkyl (e.g., C₁-C₆ heteroalkyl), optionally substituted alkenyl (e.g., C₂-C₆ alkenyl), optionally substituted heteroalkenyl (e.g., C₂-C₆ heteroalkenyl), optionally substituted alkynyl (e.g., C₂-C₆ alkynyl), optionally substituted heteroalkynyl (e.g., C₂-C₆ heteroalkynyl), optionally substituted cycloalkyl, optionally substituted heterocycloalkyl, optionally substituted aryl, or optionally substituted heteroaryl;

L is a linker, such as optionally substituted alkylene (e.g., C₁-C₆ alkylene), optionally substituted heteroalkylene (C₁-C₆ heteroalkylene), optionally substituted alkenylene (e.g., C₂-C₆ alkenylene), optionally substituted heteroalkenylene (e.g., C₂-C₆ heteroalkenylene), optionally substituted alkynylene (e.g., C₂-C₆ alkynylene), optionally substituted heteroalkynylene (e.g., C₂-C₆ heteroalkynylene), optionally substituted cycloalkylene, optionally substituted heterocycloalkylene, optionally substituted arylene, or optionally substituted heteroarylene; a dipeptide, —(C═O)—, a peptide, or a combination thereof; and

Z is a chemical moiety formed from a coupling reaction between a reactive substituent present on L and a reactive substituent present within an antibody, or an antigen-binding fragment thereof, that binds CD134 or CD278.

In some embodiments, Am contains exactly one R_{C} substituent.

In some embodiments, the linker L and the chemical moiety Z, taken together as L-Z, is

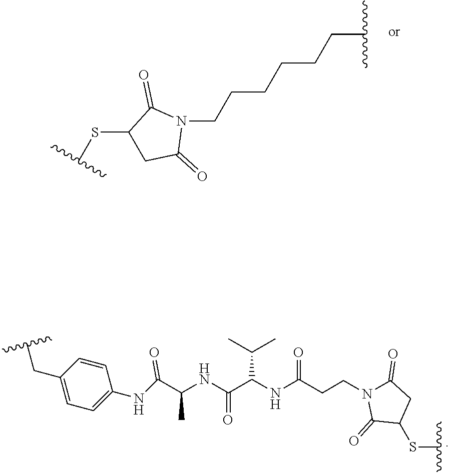

where S is a sulfur atom which represents the reactive substituent present within the antibody or antigen-binding fragment thereof, that binds CD134 or CD278 (e.g., from the —SH group of a cysteine residue).

In some embodiments, L-Z is

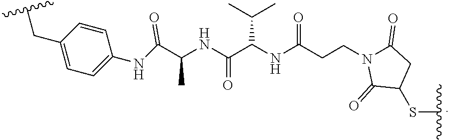

In some embodiments, Am-L-Z-Ab is:

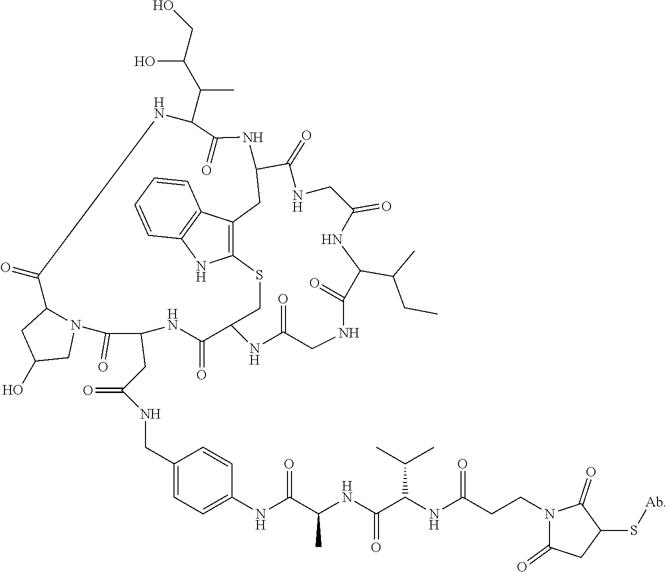

In some embodiments, Am-L-Z is represented by formula (IA)

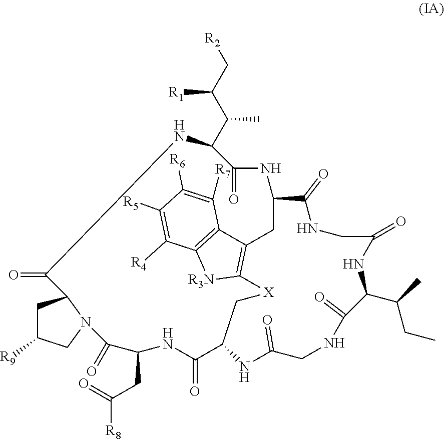

wherein R₁ is H, OH, OR_{A}, or OR_{C};

R₂ is H, OH, OR_{B}, or OR_{C};

R_{A} and R_{B}, when present, together with the oxygen atoms to which they are bound, combine to form an optionally substituted 5-membered heterocycloalkyl group;

R₃ is H, R_{C}, or R_{D};

R₄ is H, OH, OR_{C}, OR_{D}, R_{C}, or R_{D};

R₅ is H, OH, OR_{C}, OR_{D}, R_{C}, or R_{D};

R₆ is H, OH, OR_{C}, OR_{D}, R_{C}, or R_{D};

R₇ is H, OH, OR_{C}, OR_{D}, R_{C}, or R_{D};

R₈ is OH, NH₂, OR_{C}, OR_{D}, NHR_{C}, or NR_{C}R_{D};

R₉ is H, OH, OR_{C}, or OR_{D};

X is —S—, —S(O)—, or —SO₂—;

R_{C} is -L-Z;

R_{D} is optionally substituted alkyl (e.g., C₁-C₆ alkyl), optionally substituted heteroalkyl (e.g., C₁-C₆ heteroalkyl), optionally substituted alkenyl (e.g., C₂-C₆ alkenyl), optionally substituted heteroalkenyl (e.g., C₂-C₆ heteroalkenyl), optionally substituted alkynyl (e.g., C₂-C₆ alkynyl), optionally substituted heteroalkynyl (e.g., C₂-C₆ heteroalkynyl), optionally substituted cycloalkyl, optionally substituted heterocycloalkyl, optionally substituted aryl, or optionally substituted heteroaryl;

L is a linker, such as optionally substituted alkylene (e.g., C₁-C₆ alkylene), optionally substituted heteroalkylene (C₁-C₆ heteroalkylene), optionally substituted alkenylene (e.g., C₂-C₆ alkenylene), optionally substituted heteroalkenylene (e.g., C₂-C₆ heteroalkenylene), optionally substituted alkynylene (e.g., C₂-C₆ alkynylene), optionally substituted heteroalkynylene (e.g., C₂-C₆ heteroalkynylene), optionally substituted cycloalkylene, optionally substituted heterocycloalkylene, optionally substituted arylene, optionally substituted heteroarylene; , a dipeptide, —(C═O)—, a peptide, or a combination thereof;

Z is a chemical moiety formed from a coupling reaction between a reactive substituent present on L and a reactive substituent present within an antibody, or an antigen-binding fragment thereof, that binds CD134 or CD278, and

wherein Am contains exactly one R_{C} substituent.

In some embodiments, the linker L and the chemical moiety Z, taken together as L-Z, is

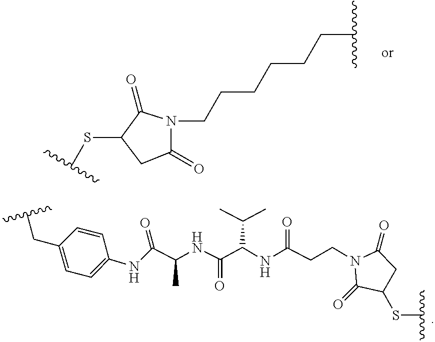

In some embodiments, L-Z is

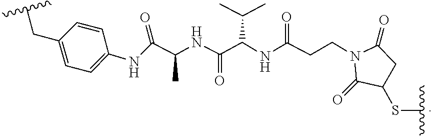

In some embodiments, Am-L-Z-Ab is

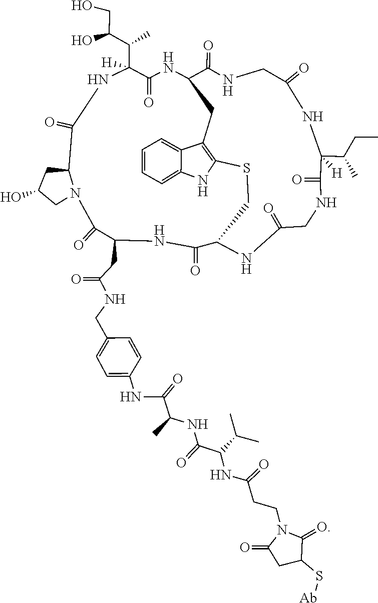

In some embodiments, Am-L-Z-Ab is

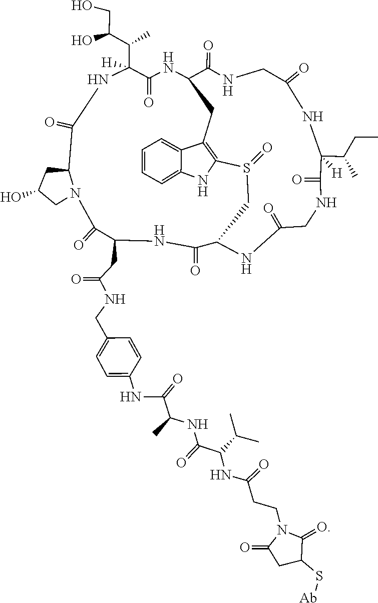

In some embodiments, Am-L-Z is represented by formula (IB)

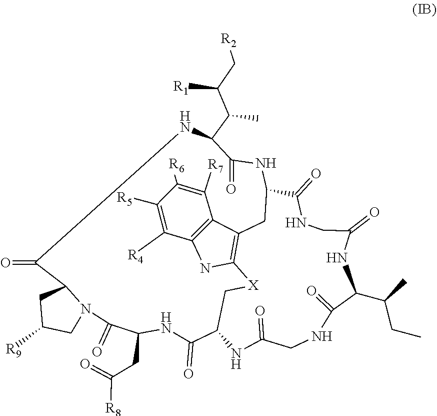

wherein R₁ is H, OH, OR_{A}, or OR_{C};

R₂ is H, OH, OR_{B}, or OR_{C};

R_{A} and R_{B}, when present, together with the oxygen atoms to which they are bound, combine to form an optionally substituted 5-membered heterocycloalkyl group;

R₃ is H, R_{C}, or R_{D};

R₄ is H, OH, OR_{C}, OR_{D}, R_{C}, or R_{D};

R₅ is H, OH, OR_{C}, OR_{D}, R_{C}, or R_{D};

R₆ is H, OH, OR_{C}, OR_{D}, R_{C}, or R_{D};

R₇ is H, OH, OR_{C}, OR_{D}, R_{C}, or R_{D};

R₈ is OH, NH₂, OR_{C}, OR_{D}, NHR_{C}, or NR_{C}R_{D};

R₉ is H, OH, OR_{C}, or OR_{D};

X is —S—, —S(O)—, or —SO₂—;

R_{C} is -L-Z;

R_{D} is optionally substituted alkyl (e.g., C₁-C₆ alkyl), optionally substituted heteroalkyl (e.g., C₁-C₆ heteroalkyl), optionally substituted alkenyl (e.g., C₂-C₆ alkenyl), optionally substituted heteroalkenyl (e.g., C₂-C₆ heteroalkenyl), optionally substituted alkynyl (e.g., C₂-C₆ alkynyl), optionally substituted heteroalkynyl (e.g., C₂-C₆ heteroalkynyl), optionally substituted cycloalkyl, optionally substituted heterocycloalkyl, optionally substituted aryl, or optionally substituted heteroaryl;

L is a linker, such as optionally substituted alkylene (e.g., C₁-C₆ alkylene), optionally substituted heteroalkylene (C₁-C₆ heteroalkylene), optionally substituted alkenylene (e.g., C₂-C₆ alkenylene), optionally substituted heteroalkenylene (e.g., C₂-C₆ heteroalkenylene), optionally substituted alkynylene (e.g., C₂-C₆ alkynylene), optionally substituted heteroalkynylene (e.g., C₂-C₆ heteroalkynylene), optionally substituted cycloalkylene, optionally substituted heterocycloalkylene, optionally substituted arylene, optionally substituted heteroarylene; , a dipeptide, —(C═O)—, a peptide, or a combination thereof;

Z is a chemical moiety formed from a coupling reaction between a reactive substituent present on L and a reactive substituent present within an antibody, or an antigen-binding fragment thereof, that binds CD134 or CD278; and

wherein Am contains exactly one R_{C} substituent.

In some embodiments, Am-L-Z is represented by formula (I), (IA), or (IB), wherein R₁ is H,

OH, OR_{A}, or OR_{C};

R₂ is H, OH, OR_{B}, or OR_{C};

R_{A} and R_{B}, when present, together with the oxygen atoms to which they are bound, combine to form:

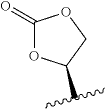 [SMILES: *[C@@H]1COC(=O)O1]

R₃ is H or R_{C};

R₄ is H, OH, OR_{C}, OR_{D}, R_{C}, or R_{D};

R₅ is H, OH, OR_{C}, OR_{D}, R_{C}, or R_{D};

R₆ is H, OH, OR_{C}, OR_{D}, R_{C}, or R_{D};

R₇ is H, OH, OR_{C}, OR_{D}, R_{C}, or R_{D};

R₈ is OH, NH₂, OR_{C}, or NHR_{C};

R₉ is H or OH; and

wherein X, R_{C} and R_{D} are each as defined above.

In some embodiments, Am-L-Z is represented by formula (I), (IA), or (IB),

wherein R₁ is H, OH, OR_{A}, or OR_{C};

R₂ is H, OH, OR_{B}, or OR_{C};

R_{A} and R_{B}, together with the oxygen atoms to which they are bound, combine to form:

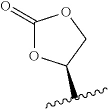 [SMILES: *[C@@H]1COC(=O)O1]

R₃ is H or R_{C};

R₄ and R₅ are each independently H, OH, OR_{C}, R_{C}, or OR_{D};

R₆ and R₇ are each H;

R₈ is OH, NH₂, OR_{C}, or NHR_{C};

R₉ is H or OH; and

wherein X and R_{C} are as defined above.

In some embodiments, Am-L-Z is represented by formula (I), (IA), or (IB),

wherein R₁ is H, OH, or OR_{A};

R₂ is H, OH, or OR_{B};

R_{A} and R_{B}, together with the oxygen atoms to which they are bound, combine to form:

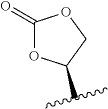 [SMILES: *[C@@H]1COC(=O)O1]

R₃, R₄, R₆, and R₇ are each H;

R₅ is OR_{C};

R₈ is OH or NH₂;

R₉ is H or OH; and

wherein X and R_{C} are as defined above.

In some embodiments, Am-L-Z is represented by formula (I), (IA), or (IB),

wherein R₁ and R₂ are each independently H or OH;

R₃ is R_{C};

R₄, R₆, and R₇ are each H;

R₅ is H, OH, or OC₁-C₆ alkyl;

R₈ is OH or NH₂;

R₉ is H or OH; and

wherein X and R_{C} are as defined above.

In some embodiments, Am-L-Z is represented by formula (I), (IA), or (IB),

wherein R₁ and R₂ are each independently H or OH;

R₃, R₆, and R₇ are each H;

R₄ and R₅ are each independently H, OH, OR_{C}, or R_{C};

R₈ is OH or NH₂;

R₉ is H or OH; and

wherein X and R_{C} are as defined above.

In some embodiments, Am-L-Z is represented by formula (I), (IA), or (IB),

wherein R₁ and R₂ are each independently H or OH;

R₃, R₆, and R₇ are each H;

R₄ and R₅ are each independently H or OH;

R₈ is OH, NH₂, OR_{C}, or NHR_{C};

R₉ is H or OH; and

wherein X and R_{C} are as defined above.

In some embodiments, the linker L and the chemical moiety Z, taken together as L-Z, is

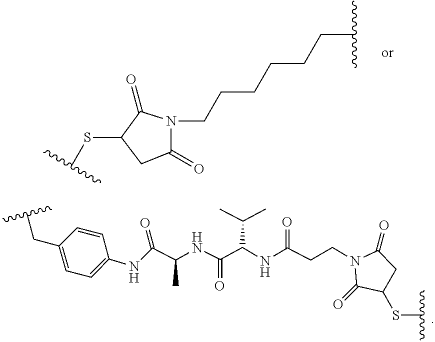

In some embodiments, L-Z is

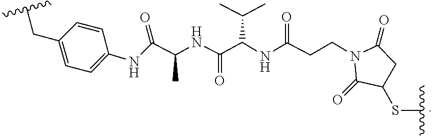

In some embodiments, Am-L-Z-Ab is

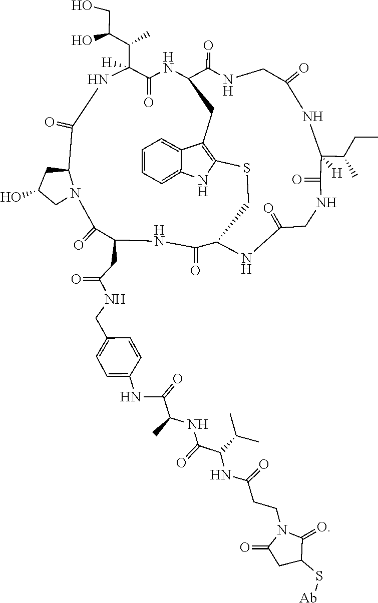

In some embodiments, Am-L-Z-Ab is

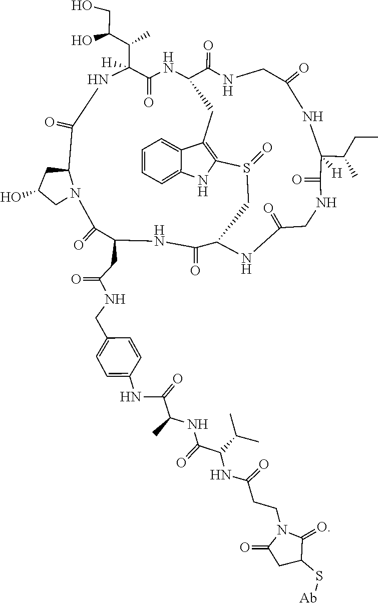

In some embodiments, the Am-L-Z-Ab precursor is

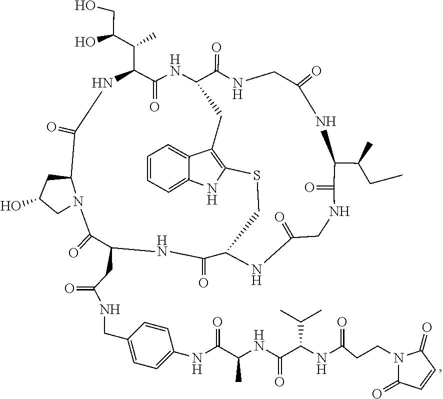 [SMILES: CC[C@H](C)[C@@H]1NC(=O)CNC(=O)[C@@H]2Cc3c([nH]c4ccccc34)SC[C@H](NC(=O)CNC1=O)C(=O)N[C@@H](CC(=O)NCc1ccc(NC(=O)[C@H](C)NC(=O)[C@@H](NC(=O)CCN3C(=O)C=CC3=O)C(C)C)cc1)C(=O)N1C[C@H](O)C[C@H]1C(=O)N[C@@H]([C@@H](C)[C@@H](O)CO)C(=O)N2]

wherein the maleimide reacts with a thiol group found on a cysteine in the antibody.

In some embodiments, the Am-L-Z-Ab precursor is

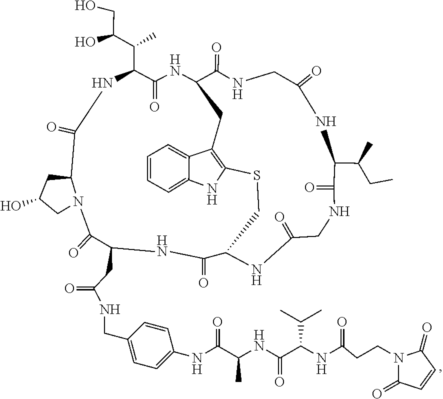 [SMILES: CC[C@H](C)[C@@H]1NC(=O)CNC(=O)[C@H]2Cc3c([nH]c4ccccc34)SC[C@H](NC(=O)CNC1=O)C(=O)N[C@@H](CC(=O)NCc1ccc(NC(=O)[C@H](C)NC(=O)[C@@H](NC(=O)CCN3C(=O)C=CC3=O)C(C)C)cc1)C(=O)N1C[C@H](O)C[C@H]1C(=O)N[C@@H]([C@@H](C)[C@@H](O)CO)C(=O)N2]

wherein the maleimide reacts with a thiol group found on a cysteine in the antibody.

In some embodiments, Am-L-Z is represented by formula (II), formula (IIA), or formula (IIB)

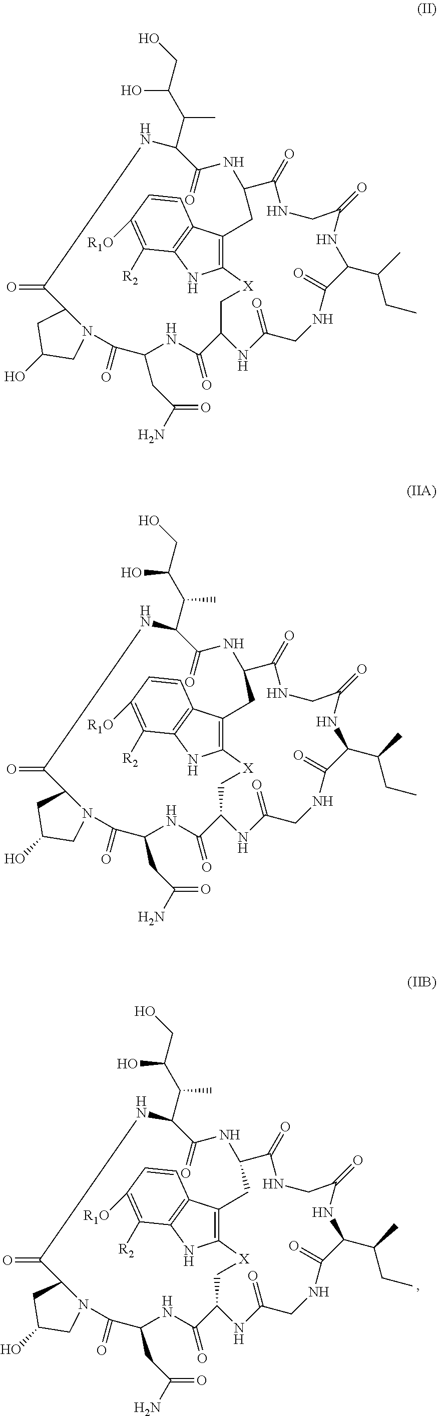

wherein X is S, SO, or SO₂; R₁ is H or a linker covalently bound to the antibody or antigen-binding fragment thereof through a chemical moiety Z, formed from a coupling reaction between a reactive substituent present on the linker and a reactive substituent present within an antibody, or antigen-binding fragment thereof; and R₂ is H or a linker covalently bound to the antibody or antigen-binding fragment thereof through a chemical moiety Z, formed from a coupling reaction between a reactive substituent present on the linker and a reactive substituent present within an antibody, or antigen-binding fragment thereof; wherein when R₁ is H, R₂ is the linker, and when R₂ is H, R₁ is the linker.

In some embodiments, the linker comprises a —(CH)₂ₙ— unit, where n is an integer from 2-6.

In some embodiments, R₁ is the linker and R₂ is H, and the linker and chemical moiety, together as L-Z, is

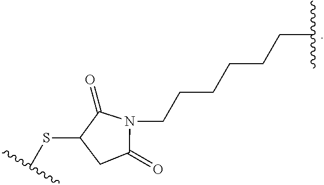 [SMILES: *CCCCCCN1C(=O)CC(S*)C1=O]

In some embodiments, Am-L-Z-Ab is one of:

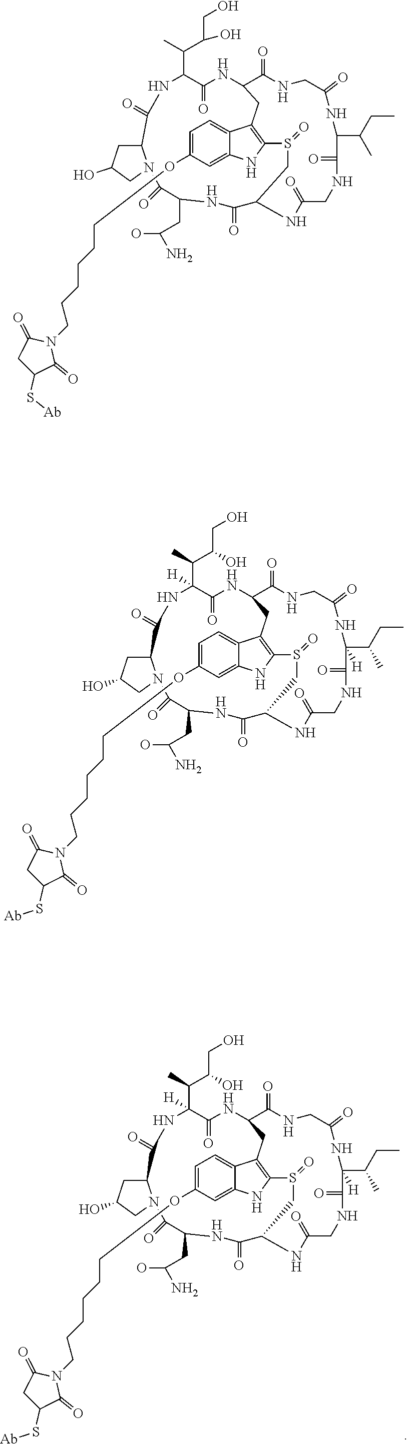

In some embodiments, the anti-CD134 or anti-CD278 antibody, antigen-binding fragment thereof, ADC, or soluble CD134 ligand is delivered into the patient prior to the patient receiving a transplant comprising hematopoietic stem cells.

In some embodiments, the anti-CD134 or anti-CD278 antibody, antigen-binding fragment thereof, ADC, or soluble CD134 ligand conjugated to a cytotoxin, such as a microtubule binding agent, is delivered into the patient about 3 days (for example, from about 1 hour to about 7 days (e.g., about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 2 days, about 3 days, about 4 days, about 5 days, about 6 days, or about 7 days) prior to administration of the hematopoietic stem cells into the patient.

In some embodiments, the anti-CD134 or anti-CD278 antibody, antigen-binding fragment thereof, ADC, or soluble CD134 ligand is delivered into the patient concomitant with the patient receiving a transplant that includes hematopoietic stem cells.

In some embodiments, the anti-CD134 or anti-CD278 antibody, antigen-binding fragment thereof, ADC, or soluble CD134 ligand is delivered into the patient after the patient receives a transplant comprising hematopoietic stem cells.

In some embodiments, the anti-CD134 or anti-CD278 antibody, the antigen-binding fragment thereof, ADC, or soluble CD134 ligand (e.g., conjugated to a cytotoxin, such as a microtubule binding agent) is delivered into the patient, for example, about 1 hour to 10 days (e.g., about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 2 days, about 3 days, about 4 days, about 5 days, about 6 days, about 7 days, about 8 days, about 9 days, or about 10 days) or more after the administration of the exogenous hematopoietic stem cell transplant. For example, the anti-CD134 or anti-CD278 antibody, antigen-binding fragment thereof, antibody-drug conjugate may be administered about 3 to 4 days after the transplant.

In some embodiments, the transplant is allogeneic. In some embodiments, the transplant is autologous.

In some embodiments, the transplant is a bone marrow transplant, a peripheral blood transplant, or a cord blood transplant.

In some embodiments, the transplant includes hematopoietic cells (e.g., hematopoietic stem cells).

In some embodiments, the hematopoietic stem cells or progeny thereof maintain hematopoietic stem cell functional potential after two or more days following transplantation of hematopoietic stem cells into the patient.

In some embodiments, the hematopoietic stem cells or progeny thereof maintain hematopoietic stem cell functional potential after two or more days (for example, from about 2 to about 5 days, from about 2 to about 7 days, from about 2 to about 20 days, from about 2 to about 30 days, such as 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or more) following transplantation of the hematopoietic stem cells into the patient.

In some embodiments, the hematopoietic stem cells or progeny thereof are capable of localizing to hematopoietic tissue, such as the bone marrow, and/or reestablishing hematopoiesis following transplantation of the hematopoietic stem cells into the patient.

In some embodiments, upon transplantation into the patient, the hematopoietic stem cells give rise to recovery of a population of cells selected from the group consisting of megakaryocytes, thrombocytes, platelets, erythrocytes, mast cells, myeoblasts, basophils, neutrophils, eosinophils, microglia, granulocytes, monocytes, osteoclasts, antigen-presenting cells, macrophages, dendritic cells, natural killer (NK) cells, T cells and B cells.

In some embodiments, the transplant comprises leukocytes.

In some embodiments, upon transplantation into the patient, the hematopoietic cells are selected from the group consisting of T cells, B cells, dendritic cells, natural killer (NK) cells, macrophages, cancer cells, neutrophils, basophils, and eosinophils.

In some embodiments, upon transplantation into the patient, the leukocytes are selected from the group consisting of T cells, B cells, dendritic cells, natural killer (NK) cells, macrophages, cancer cells, neutrophils, basophils, and eosinophils.

In some embodiments, the CD134 positive cells are selected from the group consisting of activated T cells, B cells, dendritic cells, NK cells, macrophages, cancer cells, neutrophils, basophils, and eosinophils. In some embodiments, the cell demonstrates reactivity against an antigen of the patient. In some embodiments, the CD278 positive cells are selected from the group consisting of activated T cells, B cells, dendritic cells, NK cells, macrophages, cancer cells, neutrophils, basophils, and eosinophils. In some embodiments, the anti-CD134 or anti-CD278 antibody, the antigen-binding fragment thereof, ADC, or soluble CD134 ligand is internalized by a T cell upon contact. In other embodiments, the anti-CD134 or anti-CD278 antibody, the antigen-binding fragment thereof, ADC, or soluble CD134 ligand promotes death or suppresses proliferation of a T cell.

In some embodiments, the invention provides a method of depleting a population of CD134+ cells in a human patient suffering from or at risk for GVHD by administering to the patient an effective amount of an antibody, or antigen-binding fragment, ADC, or soluble CD134 ligand capable of binding CD134 and is conjugated to a cytotoxin such as a microtubule-binding agent, wherein the hematopoietic cells comprising CD134+ cells are selected from the group consisting of T cells, B cells, dendritic cells, natural killer (NK) cells, macrophages, cancer cells, neutrophils, basophils, and eosinophils.

In some embodiments, the invention provides a method of depleting a population of CD278+ cells in a human patient suffering from or at risk for GVHD by administering to the patient an effective amount of an antibody, or antigen-binding fragment, ADC, or soluble CD278 ligand capable of binding CD278 and is conjugated to a cytotoxin such as a microtubule-binding agent, wherein the hematopoietic cells comprising CD278+ cells are selected from the group consisting of T cells, B cells, dendritic cells, natural killer (NK) cells, macrophages, cancer cells, neutrophils, basophils, and eosinophils.

In some embodiments, CD134+ cells selected from the group consisting of T cells, B cells, dendritic cells, natural killer (NK) cells, macrophages, cancer cells, neutrophils, basophils, and eosinophils demonstrate reactivity against an antigen of the patient.

In some embodiments, CD278+ cells selected from the group consisting of T cells, B cells, dendritic cells, natural killer (NK) cells, macrophages, cancer cells, neutrophils, basophils, and eosinophils demonstrate reactivity against an antigen of the patient.

In some embodiments, the antibody, antigen-binding fragment thereof, ADC, or soluble CD134 ligand is internalized by a CD134+ cell following administration to the patient. For instance, the antibody, antigen-binding fragment thereof, ADC, or soluble CD134 ligand may be internalized by a CD134+ T cell by receptor mediated endocytosis (e.g., upon binding to cell-surface CD134). In some embodiments, a cytotoxin covalently bound to the anti-CD134 antibody, antigen-binding fragment thereof, or ADC, may be released intracellularly by chemical cleavage (for instance, by enzymatic or non-specific cleavage of a linker described herein). The cytotoxin may then access its intracellular target (such as the mitotic spindle apparatus, nuclear DNA, ribosomal RNA, or topoisomerases, among others) so as to promote the death of a CD134+ T cell.

In some embodiments, the antibody, antigen-binding fragment thereof, ADC, or soluble CD278 ligand is internalized by a CD278+ cell following administration to the patient. For instance, the antibody, antigen-binding fragment thereof, ADC, or soluble CD278 ligand may be internalized by a CD278+ T cell by receptor mediated endocytosis (e.g., upon binding to cell-surface CD278). In some embodiments, a cytotoxin covalently bound to the anti-CD278 antibody, antigen-binding fragment thereof, or ADC, may be released intracellularly by chemical cleavage (for instance, by enzymatic or non-specific cleavage of a linker described herein). The cytotoxin may then access its intracellular target (such as the mitotic spindle apparatus, nuclear DNA, ribosomal RNA, or topoisomerases, among others) so as to promote the death of a CD278+ T cell.

In some embodiments, the anti-CD134 antibody, antigen-binding fragment thereof, or ADC, or soluble CD134 ligand is capable of promoting mitotic arrest and suppressing proliferation (for instance, by suppressing microtubule dynamic instability) of the CD134+ T cell. In other embodiments, the anti-CD278 antibody, antigen-binding fragment thereof, or ADC, or soluble CD278 ligand is capable of promoting mitotic arrest and suppressing proliferation (for instance, by suppressing microtubule dynamic instability) of the CD278+ T cell.

In some embodiments, the anti-CD134 antibody, antigen-binding fragment thereof, ADC, or soluble CD134 ligand may promote the death of a cell by recruiting one or more complement proteins, natural killer (NK) cells, macrophages, neutrophils, and/or eosinophils upon administration to the patient. In some embodiments, the recruitment is to a T cell. In some embodiments, the anti-CD278 antibody, antigen-binding fragment thereof, ADC, or soluble CD278 ligand may promote the death of a cell by recruiting one or more complement proteins, natural killer (NK) cells, macrophages, neutrophils, and/or eosinophils upon administration to the patient. In some embodiments, the recruitment is to a T cell.

In some embodiments, the anti-CD134 antibody, antigen-binding fragment thereof, ADC, or soluble CD134 ligand may promote the death of a CD134+ T cell by recruiting one or more complement proteins, natural killer (NK) cells, macrophages, neutrophils, and/or eosinophils upon administration to the patient. In some embodiments, the anti-CD278 antibody, antigen-binding fragment thereof, ADC, or soluble CD278 ligand may promote the death of a CD278+ T cell by recruiting one or more complement proteins, natural killer (NK) cells, macrophages, neutrophils, and/or eosinophils upon administration to the patient.

In some embodiments, the antibody or antigen-binding fragment thereof, antibody-drug conjugate, or soluble CD134 ligand is used to treat a T- or B cell-driven autoimmune disease. In some embodiments, the autoimmune disease is multiple sclerosis, rheumatoid arthritis, intestinal bowel disease, psoriasis, lupus, or Type 1 diabetes.

In some embodiments, the invention provides a method of depleting a population of CD278+ cells in a human patient suffering from or at risk for GVHD by administering to the patient an effective amount of an antibody, or antigen-binding fragment, conjugated to a cytotoxin such as a microtubule-binding agent. In certain embodiments, the hematopoietic cells comprise T cells.

In some embodiments, the anti-CD278 antibody, antigen-binding fragment thereof, or ADC is internalized by a CD278+ cell following administration to the patient. For instance, the anti-CD278 antibody, antigen-binding fragment thereof, ADC, may be internalized by a CD278+ T cell by receptor mediated endocytosis (e.g., upon binding to cell-surface CD278). In some embodiments, a cytotoxin covalently bound to the antibody, antigen-binding fragment thereof, or ADC, may be released intracellularly by chemical cleavage (for instance, by enzymatic or non-specific cleavage of a linker described herein). The cytotoxin may then access its intracellular target (such as the mitotic spindle apparatus, nuclear DNA, ribosomal RNA, or topoisomerases, among others) so as to promote the death of a CD278+ T cell.

In some embodiments, the anti-CD278 antibody, antigen-binding fragment thereof, or ADC, is capable of promoting mitotic arrest and suppressing proliferation (for instance, by suppressing microtubule dynamic instability) of the CD278+ T cell.

In some embodiments, the anti-CD278 antibody or antigen-binding fragment thereof, antibody-drug conjugate, or ADC thereof is used to treat a T- or B cell-driven autoimmune disease.

In some embodiments, the method is used to treat one or more disorders or cancers in a patient, such as a patient that has received a transplant comprising hematopoietic stem cells. For instance, the patient may be one that is suffering from a stem cell disorder. In some embodiments, the patient is suffering from a hemoglobinopathy disorder, such as sickle cell anemia, thalassemia, Fanconi anemia, and Wiskott-Aldrich syndrome. The patient may be suffering from an immunodeficiency disorder, such as a congenital immunodeficiency disorder or an acquired immunodeficiency disorder (e.g., human immunodeficiency virus or acquired immune deficiency syndrome). In some embodiments, the patient is suffering from a metabolic disorder, such as glycogen storage diseases, mucopolysaccharidoses, Gaucher's Disease, Hurlers Disease, sphingolipidoses, and metachromatic leukodystrophy. In some embodiments, the patient is suffering from cancer, such as leukemia, lymphoma, multiple myeloma and myelodysplastic syndrome, and neuroblastoma. In some embodiments, the patient is suffering from a disorder selected from the group consisting of adenosine deaminase deficiency and severe combined immunodeficiency, hyper immunoglobulin M syndrome, Chediak-Higashi disease, hereditary lymphohistiocytosis, osteopetrosis, osteogenesis imperfecta, storage diseases, thalassemia major, systemic sclerosis, systemic lupus erythematosus, multiple sclerosis, and juvenile rheumatoid arthritis. In some embodiments, the patient has received a transplant comprising a population of hematopoietic stem cells. In other embodiments, the method treats the disorder of cancer.

In a further aspect, the invention features a method of treating, preventing or reducing graft versus host disease (GVHD) in a human patient in need thereof, the method comprising administering an anti-CD134 antibody drug conjugate (ADC) to the human patient such that GVHD is prevented, wherein the ADC comprises an anti-CD134 antibody linked to a cytotoxin. In one embodiment, the cytotoxin is a microtubule-binding agent or an RNA polymerase inhibitor. In one embodiment, the method comprises administering the ADC to the patient prior to the patient receiving a transplant comprising hematopoietic stem cells. In another embodiment, the method comprising administering the ADC to the patient about three days prior to the patient receiving a transplant comprising hematopoietic stem cells. In another embodiment, the method comprises administering the ADC to the patient concomitant with the patient receiving a transplant comprising hematopoietic stem cells. In a further embodiment, the method comprises administering the ADC to the patient after the patient receives a transplant comprising hematopoietic stem cells. In yet another embodiment, the method comprising administering the ADC to the patient about 1 hour to about 10 days (e.g., about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 2 days, about 3 days, about 4 days, about 5 days, about 6 days, about 7 days, about 8 days, about 9 days, or about 10 days) after the patient receives a transplant comprising hematopoietic stem cells. In a further embodiment, the method comprising administering the ADC to the patient about 3 to 4 days after the patient receives a transplant comprising hematopoietic stem cells. In other embodiments, the transplant is allogeneic.

In yet another aspect, the invention features a method of depleting a population of CD134 positive cells in a human subject having GVHD or at risk of developing GVHD, the method comprising administering an anti-CD134 ADC to the human patient such that the population of CD134 cells is depleted, wherein the ADC comprises an anti-CD134 antibody linked to a cytotoxin. In one embodiment, the method comprises administering the ADC to the patient prior to the patient receiving a transplant comprising hematopoietic stem cells. In another embodiment, the method comprising administering the ADC to the patient about three days prior to the patient receiving a transplant comprising hematopoietic stem cells. In another embodiment, the method comprises administering the ADC to the patient concomitant with the patient receiving a transplant comprising hematopoietic stem cells. In a further embodiment, the method comprises administering the ADC to the patient after the patient receives a transplant comprising hematopoietic stem cells. In yet another embodiment, the method comprising administering the ADC to the patient about 1 hour to 10 days (e.g., about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 2 days, about 3 days, about 4 days, about 5 days, about 6 days, about 7 days, about 8 days, about 9 days, or about 10 days) after the patient receives a transplant comprising hematopoietic stem cells. In a further embodiment, the method comprising administering the ADC to the patient about 3 to 4 days after the patient receives a transplant comprising hematopoietic stem cells. In other embodiments, the transplant is allogeneic.

In a further aspect, the invention features a method of treating ,preventing or reducing graft versus host disease (GVHD) in a human patient in need thereof, the method comprising administering an anti-CD278 antibody drug conjugate (ADC) to the human patient such that GVHD is prevented, wherein the ADC comprises an anti-CD278 antibody linked to a cytotoxin. In one embodiment, the method comprises administering the ADC to the patient prior to the patient receiving a transplant comprising hematopoietic stem cells. In another embodiment, the method comprising administering the ADC to the patient about three days prior to the patient receiving a transplant comprising hematopoietic stem cells. In another embodiment, the method comprises administering the ADC to the patient concomitant with the patient receiving a transplant comprising hematopoietic stem cells. In a further embodiment, the method comprises administering the ADC to the patient after the patient receives a transplant comprising hematopoietic stem cells. In yet another embodiment, the method comprising administering the ADC to the patient about 1 hour to 10 days (e.g., e.g., about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 2 days, about 3 days, about 4 days, about 5 days, about 6 days, about 7 days, about 8 days, about 9 days, or about 10 days) after the patient receives a transplant comprising hematopoietic stem cells. In a further embodiment, the method comprising administering the ADC to the patient about 3 to 4 days after the patient receives a transplant comprising hematopoietic stem cells. In other embodiments, the transplant is allogeneic.

In yet another aspect, the invention features a method of depleting a population of CD278 positive cells in a human subject having GVHD or at risk of developing GVHD, the method comprising administering an anti-CD278 ADC to the human patient such that the population of CD278 cells is depleted, wherein the ADC comprises an anti-CD278 antibody linked to a cytotoxin. In one embodiment, the method comprises administering the ADC to the patient prior to the patient receiving a transplant comprising hematopoietic stem cells. In another embodiment, the method comprising administering the ADC to the patient about three days prior to the patient receiving a transplant comprising hematopoietic stem cells. In another embodiment, the method comprises administering the ADC to the patient concomitant with the patient receiving a transplant comprising hematopoietic stem cells. In a further embodiment, the method comprises administering the ADC to the patient after the patient receives a transplant comprising hematopoietic stem cells. In yet another embodiment, the method comprising administering the ADC to the patient about 1 hour to 10 days (e.g., e.g., about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 2 days, about 3 days, about 4 days, about 5 days, about 6 days, about 7 days, about 8 days, about 9 days, or about 10 days) after the patient receives a transplant comprising hematopoietic stem cells. In a further embodiment, the method comprising administering the ADC to the patient about 3 to 4 days after the patient receives a transplant comprising hematopoietic stem cells. In other embodiments, the transplant is allogeneic.

In yet another aspect, the invention features a method treating allograft rejection in a human patient in need thereof by administering an anti-CD134 antibody drug conjugate (ADC) to the human patient such that GVHD is treated, wherein the ADC comprises an anti-CD134 antibody linked to a cytotoxin which is a microtubule-binding agent or an RNA polymerase inhibitor.

In yet another aspect, the invention features a method treating allograft rejection in a human patient in need thereof by administering an anti-CD278 antibody drug conjugate (ADC) to the human patient such that GVHD is treated, wherein the ADC comprises an anti-CD278 antibody linked to a cytotoxin which is a microtubule-binding agent or an RNA polymerase inhibitor.

In yet another aspect, the invention features a method depleting a population of CD134 positive cells in a human subject having allograft rejection or at risk of developing allograft rejection, by administering an anti-CD134 ADC to the human patient such that the population of CD134 cells is depleted, wherein the ADC comprises an anti-CD134 antibody linked to a cytotoxin which is a microtubule-binding agent or an RNA polymerase inhibitor.

In yet another aspect, the invention features a method depleting a population of CD134 positive cells in a human subject having allograft rejection or at risk of developing allograft rejection, by administering an anti-CD278 ADC to the human patient such that the population of CD278 cells is depleted, wherein the ADC comprises an anti-CD278 antibody linked to a cytotoxin which is a microtubule-binding agent or an RNA polymerase inhibitor.

In yet another aspect, the invention features an antibody drug conjugate (ADC) comprising an anti-CD134 antibody (or an anti-CD278 antibody) conjugated to a cytotoxin via a peptide linker, wherein the cytotoxin is a microtubule-binding agent or an RNA polymerase inhibitor. In some embodiments, the RNA polymerase inhibitor is an amatoxin. In other embodiments, the amatoxin is an amanitin. In yet other embodiments, the amanitin is selected from the group consisting of α-amanitin, β-amanitin, γ-amanitin, ε-amanitin, amanin, amaninamide, amanullin, amanullinic acid, and proamanullin.

In certain embodiments, the ADC of the invention is of formula Ab-Z-L-Am, wherein Ab is an anti-CD278 antibody (or an anti-CD278 antibody), L is a linker, Z is a chemical moiety, and Am is the amatoxin, and wherein Am-L-Z is represented by formula (I)

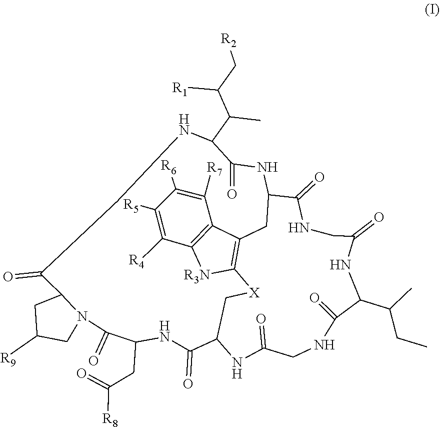

wherein R₁ is H, OH, OR_{A}, or OR_{C};

R₂ is H, OH, OR_{B}, or OR_{C};

R_{A} and R_{B}, when present, together with the oxygen atoms to which they are bound, combine to form an optionally substituted 5-membered heterocycloalkyl group;

R₃ is H, R_{C}, or R_{D};

R₄ is H, OH, OR_{C}, OR_{D}, R_{C}, or R_{D};

R₅ is H, OH, OR_{C}, OR_{D}, R_{C}, or R_{D};

R₆ is H, OH, OR_{C}, OR_{D}, R_{C}, or R_{D};

R₇ is H, OH, OR_{C}, OR_{D}, R_{C}, or R_{D};

R₈ is OH, NH₂, OR_{C}, OR_{D}, NHR_{C}, or NR_{C}R_{D};

R₉ is H, OH, OR_{C}, or OR_{D};

X is —S—, —S(O)—, or —SO₂—;

R_{C} is -L-Z;

R_{D} is optionally substituted alkyl (e.g., C₁-C₆ alkyl), optionally substituted heteroalkyl (e.g., C₁-C₆ heteroalkyl), optionally substituted alkenyl (e.g., C₂-C₆ alkenyl), optionally substituted heteroalkenyl (e.g., C₂-C₆ heteroalkenyl), optionally substituted alkynyl (e.g., C₂-C₆ alkynyl), optionally substituted heteroalkynyl (e.g., C₂-C₆ heteroalkynyl), optionally substituted cycloalkyl, optionally substituted heterocycloalkyl, optionally substituted aryl, or optionally substituted heteroaryl; and

L is a linker, such as optionally substituted alkylene (e.g., C₁-C₆ alkylene), optionally substituted heteroalkylene (C₁-C₆ heteroalkylene), optionally substituted alkenylene (e.g., C₂-C₆ alkenylene), optionally substituted heteroalkenylene (e.g., C₂-C₆ heteroalkenylene), optionally substituted alkynylene (e.g., C₂-C₆ alkynylene), optionally substituted heteroalkynylene (e.g., C₂-C₆ heteroalkynylene), optionally substituted cycloalkylene, optionally substituted heterocycloalkylene, optionally substituted arylene, or optionally substituted heteroarylene; a dipeptide, —(C═O)—, a peptide, or a combination thereof; and

Z is a chemical moiety formed from a coupling reaction between a reactive substituent present on L and a reactive substituent present within the antibody, or an antigen-binding fragment thereof, that binds CD278. In other embodiments, the ADC is of formula Ab-Z-L-Am, wherein Ab is an anti-CD278 antibody (or an anti-CD278 antibody), L is a linker, Z is a chemical moiety, and Am is the amatoxin, and wherein Am-L-Z-Ab is represented by

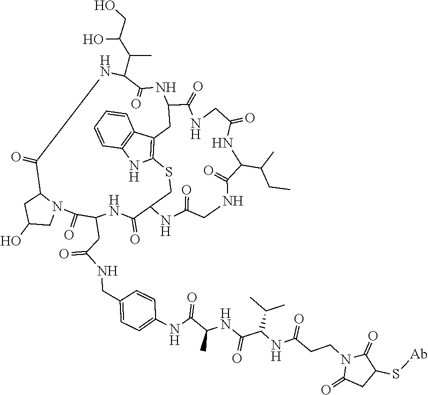

In certain embodiments, the anti-CD134 antibody is antibody BER-ACT35, antibody 443318, or antibody 7D6 as described herein. In certain embodiments, the anti-CD278 antibody is antibody DX29 or antibody 669238 as described herein.

In yet another aspect, the invention features a pharmaceutical composition including an antibody drug conjugate (ADC) comprising an anti-CD134 antibody (or an anti-CD278 antibody) conjugated to a cytotoxin via a peptide linker, wherein the cytotoxin, and a pharmaceutically active carrier.

In yet another aspect, the invention features a method of treating graft failure or GVHD in a human patient in need thereof, by administering an effective amount of an ADC as described herein to the human patient, wherein the human patient previously received a transplant. In some embodiments, the human patient received the transplant no more than 4 days prior to the administration of the ADC.

In yet another aspect, the invention features a method of treating human patient at risk of having graft failure or GVHD, by administering an effective amount of an ADC as described herein to the human patient at risk of having graft failure or GVHD, and subsequently administering a transplant to the human subject. In some embodiments, the ADC is administered to the human patient as a single dose.

### BRIEF DESCRIPTION OF THE FIGURES

FIG. 1 graphically depicts results of a flow cytometry assay measuring the expression of CD134 on both activated and resting regulatory T cells (Tregs). The results show that at 24 hours post activation, T cells were 56.9% positive for CD134 compared to the control (0 hours activation).

FIG. 2 graphically depicts results of a Treg flow analysis of fresh whole blood from three individual healthy donor controls showing CD134 is expressed on activated T cells but not significantly expressed on resting T cells.

FIG. 3 graphically depict results of an in vitro cell binding assay showing anti-CD134 antibodies binding to activated T cells.

FIGS. 4A-4D graphically depict results of an in vitro cell binding assay showing anti-CD278 antibodies (FIG. 4A) and anti-CD134 antibodies (FIG. 4C) binding to activated T cells. FIG. 4B and FIG. 4D show the same results, respectively, in comparison to an anti-CD45 positive control.

FIG. 5 graphically depict results of an in vitro T cell killing assay including an anti-CD134-amanitin ADC (i.e., “CD134-Amanitin”) and an anti-CD278-amanitin ADC (i.e., “CD278-Amanitin”) in comparison to a negative control (i.e., “hIgG-Amanitin”). The results show the number of viable activated T cells (y-axis) as a function of antibody concentration (x-axis).

FIG. 6A graphically depicts results of an in vitro T cell killing assay including an anti-CD134-amanitin ADC (i.e., “CD134-ACT35-mIgG1-Amanitin”) and anti-CD278-amanitin ADCs (i.e., “CD278-DX29-mIgG1-Amanitin” and “CD278-669238-mIgG1-Amanitin”) in comparison to a negative control (i.e., “hIgG-Amanitin”). The results show the number of viable activated T cells (y-axis) as a function of antibody concentration (x-axis).

FIG. 6B graphically depicts results of an in vitro T cell killing assay including an anti-CD134-MMAF ADC (i.e., “CD134-ACT35-mIgG1-MMAF”) and anti-CD278-MMAF ADCs (i.e., “CD278-DX29-mIgG1-MMAF” and “CD278-669238-mIgG1-MMAF”) in comparison to a negative control (i.e., “hIgG-MMAF”). The results show the number of viable active T cells (y-axis) as a function of antibody concentration (x-axis).

FIGS. 7A-7B graphically depict results of in vitro T cell killing assays including certain positive and negative control antibodies (FIG. 7A) and an anti-CD134 ADC and certain anti-CD278 ADCs in combination with Fab-SAP (saporin) (FIG. 7B). The results show the number of viable activated (blasting) T cells (y-axis) as a function of antibody concentration (x-axis).

### DEFINITIONS

As used herein, the term “about” refers to a value that is within 10% above or below the value being described. For example, the term “about 5 nM” indicates a range of from 4.5 nM to 5.5 nM.

As used herein, the term “allogeneic” refers to cells or tissues from individuals belonging to the same species but genetically different, and are therefore immunologically incompatible. Thus, the term “allogeneic cells” refers to cell types that are genetically distinct, yet belonging to the same species. Typically, the term “allogeneic” is used to define cells, such as stem cells, that are transplanted from a donor to a recipient of the same species.

As used herein, the term “amatoxin” refers to a member of the amatoxin family of peptides produced by Amanita phalloides mushrooms, or a variant or derivative thereof, such as a variant or derivative thereof capable of inhibiting RNA polymerase II activity. Also included are synthetic amatoxins (see, e.g., U.S. Pat. No. 9676702, incorporated by reference herein). Amatoxins useful in conjunction with the compositions and methods described herein include α-amanitin, β-amanitin, γ-amanitin, ε-amanitin, amanin, amaninamide, amanullin, amanullinic acid, and proamanullin, as well as derivatives thereof, such as described by formula (III) , (IIIA), or (IIIB). as described herein. As described herein, amatoxins may be conjugated to an antibody, or antigen-binding fragment thereof, that binds CD134 or CD278, for instance, by way of a linker moiety (L) (thus forming a conjugate (also referred to as an antibody drug conjugate (ADC)). Such ADCs are represented by the formula Ab-Z-L-Am, wherein Ab is the antibody or antigen-binding fragment thereof, L is a linker, Z is a chemical moiety, and Am is the amatoxin. In some embodiments, the amatoxin is conjugated to a linker. In some embodiments, the amatoxin-linker conjugate Am-L-Z is represented by formulae (I), (IA), (IB), (II), (IIA) or (IIB). Exemplary methods of amatoxin conjugation and linkers useful for such processes are described herein below. Exemplary linker-containing amatoxins useful for conjugation to an antibody, or antigen-binding fragment in accordance with the compositions and methods, are also described herein.

The term “antagonist” as used herein describes any molecule that inhibits or reduces the biological activity of a target molecule, e.g., CD134 or CD278.

As used herein, the term “antibody” refers to an immunoglobulin molecule that specifically binds to, or is immunologically reactive with, a particular antigen, and includes polyclonal, monoclonal, genetically engineered, and otherwise modified forms of antibodies, including but not limited to chimeric antibodies, humanized antibodies, heteroconjugate antibodies (e.g., bi-, tri-, and tetra-specific antibodies, diabodies, triabodies, and tetrabodies), and antigen binding fragments of antibodies, including, for example, Fab′, F(ab′)₂, Fab, Fv, rIgG, and scFv fragments. Unless otherwise indicated, the term “monoclonal antibody” (mAb) is meant to include both intact molecules, as well as antibody fragments thereof (including, for example, Fab and F(ab′)₂ fragments) that are capable of specifically binding to a target protein. As used herein, the Fab and F(ab′)₂ fragments refer to antibody fragments that lack the Fc fragment of an intact antibody. Examples of these antibody fragments are described herein.

Depending on the amino acid sequences of the constant domains of the heavy chains of antibodies, antibodies can be assigned to different classes. There are five major classes of immunoglobulins: IgA, IgD, IgE, IgG and IgM, and several of these may be further divided into subclasses (isotypes), e.g., IgG1, IgG2, IgG3, IgG4, IgA1, and IgA2. The heavy chain constant domains that correspond to the different classes of immunoglobulins are called alpha, delta, epsilon, gamma, and mu, respectively. The subunit structures and three-dimensional configurations of different classes of immunoglobulins are well known and described generally in, for example, Abbas et al. Cellular and Mol. Immunology, 4th ed. (2000). An antibody may be part of a larger fusion molecule, formed by covalent or non-covalent association of the antibody with one or more other proteins or peptides.

The term “antigen-binding fragment,” as used herein, refers to one or more fragments of an antibody that retain the ability to specifically bind to a target antigen. The antigen-binding function of an antibody can be performed by fragments of a full-length antibody. The antibody fragments can be, for example, a Fab, F(ab′)₂, scFv, diabody, a triabody, an affibody, a nanobody, an aptamer, or a domain antibody. Examples of binding fragments encompassed of the term “antigen-binding fragment” of an antibody include, but are not limited to: (i) a Fab fragment, a monovalent fragment consisting of the V_{L}, V_{H}, C_{L}, and C_{H}1 domains; (ii) a F(ab′)₂ fragment, a bivalent fragment comprising two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody, (v) a dAb including V_{H} and V_{L} domains; (vi) a dAb fragment that consists of a V_{H} domain (see, e.g., Ward et al., Nature 341:544-546, 1989); (vii) a dAb which consists of a V_{H} or a V_{L} domain; (viii) an isolated complementarity determining region (CDR); and (ix) a combination of two or more (e.g., two, three, four, five, or six) isolated CDRs which may optionally be joined by a synthetic linker. Furthermore, although the two domains of the Fv fragment, V_{L} and V_{H}, are coded for by separate genes, they can be joined, using recombinant methods, by a linker that enables them to be made as a single protein chain in which the V_{L} and V_{H} regions pair to form monovalent molecules (known as single chain Fv (scFv). These antibody fragments can be obtained using conventional techniques known to those of skill in the art, and the fragments can be screened for utility in the same manner as intact antibodies. Antigen-binding fragments can be produced by recombinant DNA techniques, enzymatic or chemical cleavage of intact immunoglobulins, or, in certain cases, by chemical peptide synthesis procedures known in the art.

As used herein, the term “anti-CD134 antibody” or an “anti-CD134 ADC” refers to an antibody, antibody fragment, or ADC that specifically binds to CD134 (also known as, e.g., OX40, OX40L Receptor, Tumor necrosis factor receptor superfamily member 4 (TNFRSF4), ACT-4, ACT35, or TXGP1L In a one embodiment, the antibody specifically binds to human CD134 (hCD134). CD134 is expressed on T-cells. The amino acid sequence of human CD134 to which an anti-CD134 antibody (or anti-CD134 conjugate) would bind is described below in SEQ ID NO: 1.

As used herein, the term “anti-CD278 antibody” or an “anti-CD278 ADC” refers to an antibody, antibody fragment, or ADC that specifically binds to CD278 (also known as ICOS). In one embodiment, the antibody specifically binds to human CD278 (hCD278). CD278 is found on T-cells. The amino acid sequence of human CD278 to which an anti-CD278 antibody (or anti-CD278 conjugate) would bind is described below in SEQ ID NO: 2.

As used herein, the term “bispecific antibody” refers to, for example, a monoclonal, often a human or humanized antibody that is capable of binding at least two different antigens. For instance, one of the binding specificities can be directed towards a T cell surface antigen, such as CD134 or CD278, the other can be for a different T cell surface antigen or another cell surface protein, such as a receptor or receptor subunit involved in a signal transduction pathway that prohibits or limits cell growth, among others.

As used herein, the term “chimeric” antibody refers to refers to antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (See, for example, U.S. Pat. No. 4,816,567 and Morrison et al., 1984, Proc. Natl. Acad. Sci. USA 81:6851-6855). In one embodiment, a chimeric antibody comprises murine heavy and light chain variable regions and human light and heavy chain constant regions.

As used herein, the terms “complementarity determining region” and “CDR” refer to a hypervariable region found both in the light chain and the heavy chain variable domains of an antibody. The more highly conserved portions of variable domains are referred to as framework regions (FRs). The amino acid positions that delineate a hypervariable region of an antibody can vary, depending on the context and the various definitions known in the art. Some positions within a variable domain may be viewed as hybrid hypervariable positions in that these positions can be deemed to be within a hypervariable region under one set of criteria while being deemed to be outside a hypervariable region under a different set of criteria. One or more of these positions can also be found in extended hypervariable regions. The antibodies described herein may contain modifications in these hybrid hypervariable positions. The variable domains of native heavy and light chains each comprise four framework regions that primarily adopt a β-sheet configuration, connected by three CDRs, which form loops that connect, and in some cases form part of, the β-sheet structure. The CDRs in each chain are held together in close proximity by the framework regions in the order FR1-CDR1-FR2-CDR2-FR3-CDR3-FR4 and, with the CDRs from the other antibody chains, contribute to the formation of the target binding site of antibodies (e.g., see Kabat et al., Sequences of Proteins of Immunological Interest, National Institute of Health, Bethesda, Md., 1987 or http://www.imgt.org/3Dstructure-DB/cgi/DomainGapAlign.cgi). Numbering of immunoglobulin amino acid residues can be performed according to the immunoglobulin amino acid residue numbering system of Kabat et al.

As used herein, the term “conjugate” refers to a compound formed by the chemical bonding of a reactive functional group of one molecule, such as an antibody or antigen-binding fragment thereof, with an appropriately reactive functional group of another molecule, such as a cytotoxin described herein. Conjugates may include a linker between the two molecules (e.g., an anti-CD134 antibody and a cytotoxin, or an anti-CD278 antibody and a cytotoxin) bound to one another, e.g., between an antibody and a cytotoxin. Examples of linkers that can be used for the formation of a conjugate include peptide-containing linkers, such as those that contain naturally occurring or non-naturally occurring amino acids, such as D-amino acids. Linkers can be prepared using a variety of strategies described herein and known in the art. Depending on the reactive components therein, a linker may be cleaved, for example, by enzymatic hydrolysis, photolysis, hydrolysis under acidic conditions, hydrolysis under basic conditions, oxidation, disulfide reduction, nucleophilic cleavage, or organometallic cleavage (see, for example, Leriche et al., Bioorg. Med. Chem., 20:571-582, 2012). Notably, the term “conjugate” (when referring to a compound) is also referred to interchangeably herein as a “drug antibody conjugate” or an “antibody drug conjugate” (ADC)”.

As used herein, the term “coupling reaction” refers to a chemical reaction in which two or more substituents suitable for reaction with one another react so as to form a chemical moiety that joins (e.g., covalently) the molecular fragments bound to each substituent. Coupling reactions include those in which a reactive substituent bound to a fragment that is a cytotoxin, such as a cytotoxin known in the art or described herein, reacts with a suitably reactive substituent bound to a fragment that is an for CD134 or CD278 known in the art or described herein. Examples of suitably reactive substituents include a nucleophile/electrophile pair (e.g., a thiol/haloalkyl pair, an amine/carbonyl pair, or a thiol/α,β-unsaturated carbonyl pair, among others), a diene/dienophile pair (e.g., an azide/alkyne pair, among others), and the like. Coupling reactions include, without limitation, thiol alkylation, hydroxyl alkylation, amine alkylation, amine condensation, amidation, esterification, disulfide formation, cycloaddition (e.g., [4+2] Diels-Alder cycloaddition, [3+2] Huisgen cycloaddition, among others), nucleophilic aromatic substitution, electrophilic aromatic substitution, and other reactive modalities known in the art or described herein.

As used herein, the term “donor” refers to a human or animal from which one or more cells are isolated prior to administration of the cells, or progeny thereof, into a recipient. The one or more cells may be, for example, a population of hematopoietic stem cells.

As used herein, the term “diabody” refers to a bivalent antibody containing two polypeptide chains, in which each polypeptide chain includes V_{H} and V_{L} domains joined by a linker that is too short (e.g., a linker composed of five amino acids) to allow for intramolecular association of V_{H} and V_{L} domains on the same peptide chain. This configuration forces each domain to pair with a complementary domain on another polypeptide chain so as to form a homodimeric structure. Accordingly, the term “triabody” refers to trivalent antibodies comprising three peptide chains, each of which contains one V_{H} domain and one V_{L} domain joined by a linker that is exceedingly short (e.g., a linker composed of 1-2 amino acids) to permit intramolecular association of V_{H} and V_{L} domains within the same peptide chain. In order to fold into their native structures, peptides configured in this way typically trimerize so as to position the V_{H} and V_{L} domains of neighboring peptide chains spatially proximal to one another (see, for example, Holliger et al., Proc. Natl. Acad. Sci. USA 90:6444-48, 1993).

The term “drug-to-antibody ratio” or “DAR” refers to the number of drugs, e.g., amatoxin, attached to the antibody of the ADC. The DAR of an ADC can range from 1 to 8, although higher loads, e.g., 10, are also possible depending on the number of linkage site on an antibody. The term DAR may be used in reference to the number of drugs loaded onto an individual antibody, or, alternatively, may be used in reference to the average or mean DAR of a group of ADCs (i.e., a “mean DAR”).

As used herein, a “dual variable domain immunoglobulin” (“DVD-Ig”) refers to an antibody that combines the target-binding variable domains of two monoclonal antibodies via linkers to create a tetravalent, dual-targeting single agent (see, for example, Gu et al., Meth. Enzymol., 502:25-41, 2012).

As used herein, the term “endogenous” describes a substance, such as a molecule, cell, tissue, or organ (e.g., a hematopoietic stem cell or a cell of hematopoietic lineage, such as a megakaryocyte, thrombocyte, platelet, erythrocyte, mast cell, myoblast, basophil, neutrophil, eosinophil, microglial cell, granulocyte, monocyte, osteoclast, antigen-presenting cell, macrophage, dendritic cell, natural killer cell, T cell, or B cell) that is found naturally in a particular organism, such as a human patient.

As used herein, the term “exogenous” describes a substance, such as a molecule, cell, tissue, or organ (e.g., a hematopoietic stem cell or a cell of hematopoietic lineage, such as a megakaryocyte, thrombocyte, platelet, erythrocyte, mast cell, myeoblast, basophil, neutrophil, eosinophil, microglial cell, granulocyte, monocyte, osteoclast, antigen-presenting cell, macrophage, dendritic cell, natural killer cell, T cell, or B cell) that is not found naturally in a particular organism, such as a human patient. Exogenous substances include those that are provided from an external source to an organism or to cultured matter extracted therefrom.

As used herein, the term “framework region”, “FR”, or “FW region” includes amino acid residues that are adjacent to the CDRs within a variable region of an antibody, or antigen-binding fragment thereof. FW region residues may be present in, for example, human antibodies, humanized antibodies, monoclonal antibodies, antibody fragments, Fab fragments, single chain antibody fragments, scFv fragments, antibody domains, and bispecific antibodies, among others.

As used herein, the term “half-life” refers to the time it takes for the plasma concentration of the antibody drug in the body to be reduced by one half or 50%. This 50% reduction in serum concentration reflects the amount of drug circulating and not removed by the natural methods of antibody clearance.

As used herein, the term “hematopoietic stem cells” (“HSCs”) refers to immature blood cells having the capacity to self-renew and to differentiate into mature blood cells comprising diverse lineages including but not limited to granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B cells and T cells). Such cells may include CD34⁺ cells. CD34⁺ cells are immature cells that express the CD34 cell surface marker. In humans, CD34+ cells are believed to include a subpopulation of cells with the stem cell properties defined above, whereas in mice, HSCs are CD34−. In addition, HSCs also refer to long term repopulating HSCs (LT-HSC) and short term repopulating HSCs (ST-HSC). LT-HSCs and ST-HSCs are differentiated, based on functional potential and on cell surface marker expression. For example, human HSCs are CD34+, CD38−, CD45RA−, CD90+, CD49F+, and lin− (negative for mature lineage markers including CD2, CD3, CD4, CD7, CD8, CD10, CD11B, CD19, CD20, CD56, CD235A). In mice, bone marrow LT-HSCs are CD34−, SCA-1+, C-kit+, CD135−, Slamfl/CD150+, CD48−, and lin− (negative for mature lineage markers including Ter119, CD11b, Gr1, CD3, CD4, CD8, B220, IL7ra), whereas ST-HSCs are CD34+, SCA-1+, C-kit+, CD135−, Slamfl/CD150+, and lin− (negative for mature lineage markers including Ter119, CD11b, Gr1, CD3, CD4, CD8, B220, IL7ra). In addition, ST-HSCs are less quiescent and more proliferative than LT-HSCs under homeostatic conditions. However, LT-HSC have greater self renewal potential (i.e., they survive throughout adulthood, and can be serially transplanted through successive recipients), whereas ST-HSCs have limited self renewal (i.e., they survive for only a limited period of time, and do not possess serial transplantation potential). Any of these HSCs can be used in the methods described herein. ST-HSCs are particularly useful because they are highly proliferative and thus, can more quickly give rise to differentiated progeny.

As used herein, the term “hematopoietic stem cell functional potential” refers to the functional properties of hematopoietic stem cells which include 1) multi-potency (which refers to the ability to differentiate into multiple different blood lineages including, but not limited to, granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, T cells and B cells), 2) self-renewal (which refers to the ability of hematopoietic stem cells to give rise to daughter cells that have equivalent potential as the mother cell, and further that this ability can repeatedly occur throughout the lifetime of an individual without exhaustion), and 3) the ability of hematopoietic stem cells or progeny thereof to be reintroduced into a transplant recipient whereupon they home to the hematopoietic stem cell niche and re-establish productive and sustained hematopoiesis.

As used herein, the term “human antibody” refers to an antibody in which substantially every part of the protein (for example, all CDRs, framework regions, C_{L}, C_{H} domains (e.g., C_{H}1, C_{H}2, C_{H}3), hinge, and V_{L} and V_{H} domains) is substantially non-immunogenic in humans, with only minor sequence changes or variations. A human antibody can be produced in vitro in a human cell (for example, by recombinant expression) or by a non-human animal or a prokaryotic or eukaryotic cell that is capable of expressing functionally rearranged human immunoglobulin (such as heavy chain and/or light chain) genes. When a human antibody is a single chain antibody, it can include a linker peptide that is not found in native human antibodies. For example, an Fv can contain a linker peptide, such as two to about eight glycine or other amino acid residues, which connects the variable region of the heavy chain and the variable region of the light chain. Such linker peptides are considered to be of human origin. Human antibodies can be made by a variety of methods known in the art including phage display methods using antibody libraries derived from human immunoglobulin sequences. Human antibodies can also be produced using transgenic mice that are incapable of expressing functional endogenous immunoglobulins, but which can express human immunoglobulin genes (see, for example, PCT Publication Nos. WO1998/24893; WO1992/01047; WO1996/34096; WO1996/33735; U.S. Pat. Nos. 5,413,923; 5,625,126; 5,633,425; 5,569,825; 5,661,016; 5,545,806; 5,814,318; 5,885,793; 5,916,771; and 5,939,598). In one embodiment, a human antibody is made using recombinant methods such that the glycosylation pattern of the antibody is different than an antibody having the same sequence if it were to exist in nature.

“Humanized” forms of non-human (e.g., murine) antibodies are chimeric antibodies that contain minimal sequence derived from non-human immunoglobulin. In one embodiment, a humanized antibody is a human antibody (recipient antibody) in which residues from CDRs of the recipient are replaced by residues from CDRs of a non-human species (donor antibody) such as mouse, rat, rabbit, or nonhuman primate having the desired specificity, affinity, and/or capacity. In some instances, framework region (FR) residues of the human antibody are replaced by corresponding non-human residues. Furthermore, humanized antibodies may comprise residues that are not found in the recipient antibody or in the donor antibody. These modifications may be made to further refine antibody performance. In general, a humanized antibody will comprise substantially all of at least one, and typically two, variable domains, in which all or substantially all of the hypervariable loops correspond to those of a non-human antibody, and all or substantially all of the FRs are those of a human immunoglobulin sequence. The humanized antibody optionally will also comprise at least a portion of an antibody constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992). See also the following review articles and references cited therein: Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994).

The terms “full length antibody” and “intact antibody” are used herein interchangeably to refer to an antibody in its substantially intact form, and not an antibody fragment as defined herein. Thus, for an IgG antibody, an intact antibody comprises two heavy chains each comprising a variable region, a constant region and an Fc region, and two light chains each comprising a variable region and a constant region. More specifically, an intact IgG comprises two light chains each comprising a light chain variable region (VL) and a light chain contact region (CL) and comprises two heavy chains each comprising a heavy chain variable region (VH) and three heavy chain constant regions (CH1, CH2, and CH3). CH2 and CH3 represent the Fc region of the heavy chain.

“Isolated,” when used herein refers to a polypeptide, e.g., an antibody, that has been identified and separated and/or recovered from a cell or cell culture from which it was expressed. Ordinarily, an isolated antibody will be prepared by at least one purification step. Thus, an “isolated antibody,” refers to an antibody which is substantially free of other antibodies having different antigenic specificities.

As used herein, the term “microtubule-binding agent” refers to a compound which acts by disrupting the microtubular network that is essential for mitotic and interphase cellular function. Examples of a microtubule-binding agent include, but are not limited to, maytasine, maytansinoids, and derivatives thereof, such as those described herein or known in the art, vinca alkaloids, such as vinblastine, vinblastine sulfate, vincristine, vincristine sulfate, vindesine, and vinorelbine, taxanes, such as docetaxel and paclitaxel, macrolides, such as discodermolides, cochicine, and epothilones, and derivatives thereof, such as epothilone B or a derivative thereof. Paclitaxel is marketed as TAXOL®; docetaxel as TAXOTERE®; vinblastine sulfate as VINBLASTIN R.P®; and vincristine sulfate as FARMISTIN®. Also included are the generic forms of paclitaxel as well as various dosage forms of paclitaxel. Generic forms of paclitaxel include, but are not limited to, betaxolol hydrochloride. Various dosage forms of paclitaxel include, but are not limited to albumin nanoparticle paclitaxel marketed as ABRAXANE®; ONXOL®, CYTOTAX®. Discodermolide can be obtained, e.g., as disclosed in U.S. Pat. No. 5,010,099. Also included are epotholine derivatives which are disclosed in U.S. Pat. No. 6,194,181, WO9810121, WO9825929, WO9808849, WO9943653, WO9822461 and WO0031247, the disclosures of each of which are incorporated herein by reference.

As used herein, the term “monoclonal antibody” refers to an antibody that is derived from a single clone, including any eukaryotic, prokaryotic, or phage clone, and not the method by which it is produced.

As used herein, the term “patient at risk for GVHD” refers to a patient with one or more risk factors for developing GVHD. Risk factors include, but are not limited to, allogeneic donor transplant (e.g., transplantation of hematopoietic stem cells from a bone marrow transplant), including mismatched human leucocyte antigen (HLA) donor and sex mismatched donor, T cell replete stem cell transplant, donor and recipient age, presence of cytomegalovirus (CMV) or CMV antibodies in transplant donor or host, increased dose of total-body irradiation (TBI), conditioning regimen intensity, acute GVHD prophylaxis, lack of protective environments, splenectomy, immunoglobulin use, underlying disease, ABO compatibility, prior exposure to herpes viruses, donor blood transfusions, performance score, antibiotic gut decontamination, and post-allogeneic transplant blood transfusions.

As used herein, the term “patient at risk for an autoimmune disease” refers to a patient with one or more risk factors for developing an autoimmune disease. Risk factors include, but are not limited to, age (young to middle aged), sex (female), ethnicity (African American, American Indian, or Latino), family history of autoimmune diseases, exposure to environmental agents, previous infection, chronic inflammation, and donor transplantation (e.g., transplantation of hematopoietic stem cells from a bone marrow transplant).

As used herein, the term “recipient” refers to a patient that receives a transplant, such as a transplant containing a population of hematopoietic stem cells. The transplanted cells administered to a recipient may be, e.g., autologous, syngeneic, or allogeneic cells.

As used herein, the term “sample” refers to a specimen (e.g., blood, blood component (e.g., serum or plasma), urine, saliva, amniotic fluid, cerebrospinal fluid, tissue (e.g., placental or dermal), pancreatic fluid, chorionic villus sample, and cells) taken from a subject.

As used herein, the term “scFv” refers to a single chain Fv antibody in which the variable domains of the heavy chain and the light chain from an antibody have been joined to form one chain. scFv fragments contain a single polypeptide chain that includes the variable region of an antibody light chain (V_{L}) (e.g., CDR-L1, CDR-L2, and/or CDR-L3) and the variable region of an antibody heavy chain (V_{H}) (e.g., CDR-H1, CDR-H2, and/or CDR-H3) separated by a linker. The linker that joins the V_{L} and V_{H} regions of a scFv fragment can be a peptide linker composed of proteinogenic amino acids. Alternative linkers can be used to so as to increase the resistance of the scFv fragment to proteolytic degradation (for example, linkers containing D-amino acids), in order to enhance the solubility of the scFv fragment (for example, hydrophilic linkers such as polyethylene glycol-containing linkers or polypeptides containing repeating glycine and serine residues), to improve the biophysical stability of the molecule (for example, a linker containing cysteine residues that form intramolecular or intermolecular disulfide bonds), or to attenuate the immunogenicity of the scFv fragment (for example, linkers containing glycosylation sites). It will also be understood by one of ordinary skill in the art that the variable regions of the scFv molecules described herein can be modified such that they vary in amino acid sequence from the antibody molecule from which they were derived. For example, nucleotide or amino acid substitutions leading to conservative substitutions or changes at amino acid residues can be made (e.g., in CDR and/or framework residues) so as to preserve or enhance the ability of the scFv to bind to the antigen recognized by the corresponding antibody.

The terms “specific binding” or “specifically binding”, as used herein, refers to the ability of an antibody (or an ADC) to recognize and bind to a specific protein structure (epitope) rather than to proteins generally. If an antibody or ADC is specific for epitope “A”, the presence of a molecule containing epitope A (or free, unlabeled A), in a reaction containing labeled “A” and the antibody, will reduce the amount of labeled A bound to the antibody or ADC. By way of example, an antibody “binds specifically” to a target if the antibody, when labeled, can be competed away from its target by the corresponding non-labeled antibody. In one embodiment, an antibody specifically binds to a target, e.g., CD134 or CD278, if the antibody has a K_{D} for the target of at least about 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, 10⁻¹² M, or less (less meaning a number that is less than 10⁻¹², e.g. 10⁻¹³). In one embodiment, the term “specific binding to CD134” or “specifically binds to CD134,” as used herein, refers to an antibody or an ADC that binds to CD134 and has a dissociation constant (K_{D}) of 1.0×10⁻⁷ M or less, as determined by surface plasmon resonance. In another embodiment, the term “specific binding to CD278” or “specifically binds to CD278,” as used herein, refers to an antibody or an ADC that binds to CD278 and has a dissociation constant (K_{D}) of 1.0×10⁻⁷ M or less, as determined by surface plasmon resonance. In one embodiment, K_{D} is determined according to standard bio-layer interferometery (BLI). It shall be understood, however, that the antibody or ADC may be capable of specifically binding to two or more antigens which are related in sequence. For example, in one embodiment, an antibody can specifically bind to both human and a non-human (e.g., mouse or non-human primate) orthologs of CD134. As another example, in one embodiment, an antibody can specifically bind to both human and a non-human (e.g., mouse or non-human primate) orthologs of CD278.

As used herein, the terms “subject” and “patient” refer to an organism, such as a human, that receives treatment for a particular disease or condition as described herein. For instance, a patient, such as a human patient, may receive treatment prior to hematopoietic stem cell transplant therapy in order to treat or prevent GVHD by administration of an antibody, antigen-binding fragment thereof, ADC, or ligand as described herein capable of binding CD134 or CD278.

As used herein, the phrase “substantially cleared from the blood” refers to a point in time following administration of a therapeutic agent (such as an anti-CD134 or an anti-CD278 antibody, antigen-binding fragment thereof, ADC, or soluble ligand) to a patient when the concentration of the therapeutic agent in a blood sample isolated from the patient is such that the therapeutic agent is not detectable by conventional means (for instance, such that the therapeutic agent is not detectable above the noise threshold of the device or assay used to detect the therapeutic agent). A variety of techniques known in the art can be used to detect antibodies, antibody fragments and protein ligands, such as ELISA-based detection assays known in the art or described herein. Additional assays that can be used to detect antibodies, antibody fragments, and protein ligands include immunoprecipitation techniques and immunoblot assays, among others known in the art.

As used herein, the phrase “stem cell disorder” broadly refers to any disease, disorder, or condition that may be treated or cured by conditioning a subject's target tissues, and/or by ablating an endogenous stem cell population in a target tissue (e.g., ablating an endogenous hematopoietic stem or progenitor cell population from a subject's bone marrow tissue) and/or by engrafting or transplanting stem cells in a subject's target tissues. For example, Type I diabetes has been shown to be cured by hematopoietic stem cell transplant and may benefit from conditioning in accordance with the compositions and methods described herein. Additional disorders that can be treated using the compositions and methods described herein include, without limitation, sickle cell anemia, thalassemias, Fanconi anemia, Wiskott-Aldrich syndrome, ADA SCID, HIV/AIDS, metachromatic leukodystrophy, Diamond-Blackfan anemia, and Schwachman-Diamond syndrome. The subject may have or be affected by an inherited blood disorder (e.g., sickle cell anemia) or an autoimmune disorder. Additionally or alternatively, the subject may have or be affected by a malignancy, such as a malignancy selected from the group consisting of hematologic cancers (e.g., leukemia, lymphoma, multiple myeloma, or myelodysplastic syndrome) and neuroblastoma. In some embodiments, the subject has or is otherwise affected by a metabolic disorder. For example, the subject may suffer or otherwise be affected by a metabolic disorder selected from the group consisting of glycogen storage diseases, mucopolysaccharidoses, Gaucher's Disease, Hurlers Disease, sphingolipidoses, metachromatic leukodystrophy, or any other diseases or disorders which may benefit from the treatments and therapies disclosed herein and including, without limitation, severe combined immunodeficiency, Wiscott-Aldrich syndrome, hyper immunoglobulin M (IgM) syndrome, Chediak-Higashi disease, hereditary lymphohistiocytosis, osteopetrosis, osteogenesis imperfecta, storage diseases, thalassemia major, sickle cell disease, systemic sclerosis, systemic lupus erythematosus, multiple sclerosis, juvenile rheumatoid arthritis and those diseases, or disorders described in “Bone Marrow Transplantation for Non-Malignant Disease,” ASH Education Book, 1:319-338 (2000), the disclosure of which is incorporated herein by reference in its entirety as it pertains to pathologies that may be treated by administration of hematopoietic stem cell transplant therapy.

As used herein, the term “suffering from disease” refers to a subject (e.g., a human) that is experiencing GVHD or an autoimmune disease. It is not intended that the present invention be limited to any particular signs or symptoms, nor disease. Thus, it is intended that the present invention encompass subjects that are experiencing any range of disease, from sub-clinical to full-blown disease, wherein the subject exhibits at least some of the indicia (e.g., signs and symptoms) associated with GVHD or an autoimmune disease.

As used herein, the term “transfection” refers to any of a wide variety of techniques commonly used for the introduction of exogenous DNA into a prokaryotic or eukaryotic host cell, such as electroporation, lipofection, calcium-phosphate precipitation, DEAE-dextran transfection and the like.

As used herein, the term “transplant” refers to any organ, body tissue, or cell(s) that has been transferred from its site of origin to a recipient site, or the act of doing so.

As used herein, the terms “prevent” or “preventing” refers to halting, delaying and/or reducing the severity of symptoms associated with a disorder, such as GVHD or an autoimmune disease.

As used herein, the terms “treat” or “treatment” refer to therapeutic treatment, in which the object is to prevent or slow down (lessen) an undesired physiological change or disorder or to promote a beneficial phenotype in the patient being treated for a disorder. Beneficial or desired clinical results include, but are not limited to, a decrease in the cell count or relative concentration of CD134 or CD278 positive cells, a decrease in the cellular and clinical manifestations of GVHD or an autoimmune disease, and/or promoting the engraftment of exogenous hematopoietic cells in a patient as described herein and subsequent hematopoietic stem cell transplant therapy. Additional beneficial results include an increase in the cell count or relative concentration of hematopoietic stem cells suffering from or at risk for GVHD. Beneficial results of therapy described herein may also include an increase in the cell count or relative concentration of one or more cells of hematopoietic lineage, such as a megakaryocyte, thrombocyte, platelet, erythrocyte, mast cell, myeoblast, basophil, neutrophil, eosinophil, microglial cell, granulocyte, monocyte, osteoclast, antigen-presenting cell, macrophage, dendritic cell, natural killer cell, T cell, or B cell, following hematopoietic stem cell transplant therapy.

As used herein, the term “effective amount” or “therapeutically effective amount” refers to an amount that is sufficient to achieve the desired result or to have an effect on GVHD or an autoimmune disease. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the art. Dosage can vary, and can be administered in one or more dose administrations daily, for one or several days.

The “variable region” or “variable domain” of an antibody refers to the amino-terminal domains of the heavy or light chain of the antibody. The variable domain of the heavy chain may be referred to as “VH.” The variable domain of the light chain may be referred to as “VL.” These domains are generally the most variable parts of an antibody and contain the antigen-binding sites (CDRs).

As used herein, the term “vector” includes a nucleic acid vector, such as a plasmid, a DNA vector, a plasmid, a RNA vector, virus, or other suitable replicon. Expression vectors described herein may contain a polynucleotide sequence as well as, for example, additional sequence elements used for the expression of proteins and/or the integration of these polynucleotide sequences into the genome of a mammalian cell. Certain vectors that can be used for the expression of antibodies and antibody fragments of the invention include plasmids that contain regulatory sequences, such as promoter and enhancer regions, which direct gene transcription. Other useful vectors for expression of antibodies and antibody fragments contain polynucleotide sequences that enhance the rate of translation of these genes or improve the stability or nuclear export of the mRNA that results from gene transcription. These sequence elements may include, for example, 5′ and 3′ untranslated regions and a polyadenylation signal site in order to direct efficient transcription of the gene carried on the expression vector. The expression vectors described herein may also contain a polynucleotide encoding a marker for selection of cells that contain such a vector. Examples of a suitable marker include genes that encode resistance to antibiotics, such as ampicillin, chloramphenicol, kanamycin, and nourseothricin.

As used herein, the term “alkyl” refers to a straight- or branched-chain alkyl group having, for example, from 1 to 20 carbon atoms in the chain. Examples of alkyl groups include methyl, ethyl, n-propyl, isopropyl, butyl, isobutyl, sec-butyl, tert-butyl, pentyl, isopentyl, tert-pentyl, hexyl, isohexyl, and the like.

As used herein, the term “alkylene” refers to a straight- or branched-chain divalent alkyl group. The divalent positions may be on the same or different atoms within the alkyl chain. Examples of alkylene include methylene, ethylene, propylene, isopropylene, and the like.

As used herein, the term “heteroalkyl” refers to a straight or branched-chain alkyl group having, for example, from 1 to 20 carbon atoms in the chain, and further containing one or more heteroatoms (e.g., oxygen, nitrogen, or sulfur, among others) in the chain.

As used herein, the term “heteroalkylene” refers to a straight- or branched-chain divalent heteroalkyl group. The divalent positions may be on the same or different atoms within the heteroalkyl chain.

As used herein, the term “alkenyl” refers to a straight- or branched-chain alkenyl group having, for example, from 2 to 20 carbon atoms in the chain. Examples of alkenyl groups include vinyl, propenyl, isopropenyl, butenyl, tert-butylenyl, hexenyl, and the like.

As used herein, the term “alkenylene” refers to a straight- or branched-chain divalent alkenyl group. The divalent positions may be on the same or different atoms within the alkenyl chain. Examples of alkenylene include ethenylene, propenylene, isopropenylene, butenylene, and the like.

As used herein, the term “heteroalkenyl” refers to a straight- or branched-chain alkenyl group having, for example, from 2 to 20 carbon atoms in the chain, and further containing one or more heteroatoms (e.g., oxygen, nitrogen, or sulfur, among others) in the chain.

As used herein, the term “heteroalkenylene” refers to a straight- or branched-chain divalent heteroalkenyl group. The divalent positions may be on the same or different atoms within the heteroalkenyl chain.

As used herein, the term “alkynyl” refers to a straight- or branched-chain alkynyl group having, for example, from 2 to 20 carbon atoms in the chain. Examples of alkynyl groups include propargyl, butynyl, pentynyl, hexynyl, and the like.

As used herein, the term “alkynylene” refers to a straight- or branched-chain divalent alkynyl group. The divalent positions may be on the same or different atoms within the alkynyl chain.

As used herein, the term “heteroalkynyl” refers to a straight- or branched-chain alkynyl group having, for example, from 2 to 20 carbon atoms in the chain, and further containing one or more heteroatoms (e.g., oxygen, nitrogen, or sulfur, among others) in the chain.

As used herein, the term “heteroalkynylene” refers to a straight- or branched-chain divalent heteroalkynyl group. The divalent positions may be on the same or different atoms within the heteroalkynyl chain.

As used herein, the term “cycloalkyl” refers to a monocyclic, or fused, bridged, or spiro polycyclic ring structure that is saturated and has, for example, from 3 to 12 carbon ring atoms. Examples of cycloalkyl groups include cyclopropyl, cyclobutyl, cyclopentyl, cyclohexyl, cycloheptyl, cyclooctyl, bicyclo[3.1.0]hexane, and the like.

As used herein, the term “cycloalkylene” refers to a divalent cycloalkyl group. The divalent positions may be on the same or different atoms within the ring structure. Examples of cycloalkylene include cyclopropylene, cyclobutylene, cyclopentylene, cyclohexylene, and the like.

As used herein, the term “heterocycloalkyl” refers to a monocyclic, or fused, bridged, or spiro polycyclic ring structure that is saturated and has, for example, from 3 to 12 ring atoms per ring structure selected from carbon atoms and heteroatoms selected from, e.g., nitrogen, oxygen, and sulfur, among others. The ring structure may contain, for example, one or more oxo groups on carbon, nitrogen, or sulfur ring members. Examples of heterocycloalkyls include, by way of example and not limitation, dihydroypyridyl, tetrahydropyridyl (piperidyl), tetrahydrothiophenyl, piperidinyl, 4-piperidonyl, pyrrolidinyl, 2-pyrrolidonyl, tetrahydrofuranyl, tetrahydropyranyl, bis-tetrahydropyranyl, tetrahydroquinolinyl, tetrahydroisoquinolinyl, decahydroquinolinyl, octahydroisoquinolinyl, piperazinyl, quinuclidinyl, and morpholinyl.

As used herein, the term “heterocycloalkylene” refers to a divalent heterocyclolalkyl group. The divalent positions may be on the same or different atoms within the ring structure.

As used herein, the term “aryl” refers to a monocyclic or multicyclic aromatic ring system containing, for example, from 6 to 19 carbon atoms. Aryl groups include, but are not limited to, phenyl, fluorenyl, naphthyl, and the like.

As used herein, the term “arylene” refers to a divalent aryl group. The divalent positions may be on the same or different atoms.

As used herein, the term “heteroaryl” refers to a monocyclic heteroaromatic, or a bicyclic or a tricyclic fused-ring heteroaromatic group in which one or more ring atoms is a heteroatom, e.g., nitrogen, oxygen, or sulfur. Heteroaryl groups include pyridyl, pyrrolyl, furyl, thienyl, imidazolyl, oxazolyl, isoxazolyl, thiazolyl, isothiazolyl, pyrazolyl, 1,2,3-triazolyl, 1,2,4-triazolyl, 1,2,3-oxadiazolyl, 1,2,4-oxadia-zolyl, 1,2,5-oxadiazolyl, 1,3,4-oxadiazolyl, 1,3,4-triazinyl, 1,2,3-triazinyl, benzofuryl, [2,3-dihydro]benzofuryl, isobenzofuryl, benzothienyl, benzotriazolyl, isobenzothienyl, indolyl, isoindolyl, 3H-indolyl, benzimidazolyl, imidazo[1,2-a]pyridyl, benzothiazolyl, benzoxazolyl, quinolizinyl, quinazolinyl, pthalazinyl, quinoxalinyl, cinnolinyl, napthyridinyl, pyrido[3,4-b]pyridyl, pyrido[3,2-b]pyridyl, pyrido[4,3-b]pyridyl, quinolyl, isoquinolyl, tetrazolyl, 5,6,7,8-tetrahydroquinolyl, 5,6,7,8-tetrahydroisoquinolyl, purinyl, pteridinyl, carbazolyl, xanthenyl, benzoquinolyl, and the like.

As used herein, the term “heteroarylene” refers to a divalent heteroaryl group. The divalent positions may be on the same or different atoms.

Unless otherwise constrained by the definition of the individual substituent, the foregoing chemical moieties, such as “alkyl”, “alkylene”, “heteroalkyl”, “heteroalkylene”, “alkenyl”, “alkenylene”, “heteroalkenyl”, “heteroalkenylene”, “alkynyl”, “alkynylene”, “heteroalkynyl”, “heteroalkynylene”, “cycloalkyl”, “cycloalkylene”, “heterocyclolalkyl”, heterocycloalkylene”, “aryl,” “arylene”, “heteroaryl”, and “heteroarylene” groups can optionally be substituted with, for example, from 1 to 5 substituents selected from the group consisting of alkyl, alkenyl, alkynyl, cycloalkyl, heterocycloalkyl, alkyl aryl, alkyl heteroaryl, alkyl cycloalkyl, alkyl heterocycloalkyl, amino, ammonium, acyl, acyloxy, acylamino, aminocarbonyl, alkoxycarbonyl, ureido, carbamate, aryl, heteroaryl, sulfinyl, sulfonyl, alkoxy, sulfanyl, halogen, carboxy, trihalomethyl, cyano, hydroxy, mercapto, nitro, and the like. Typical substituents include, but are not limited to, —X, —R, —OH, —OR, —SH, —SR, NH₂, —NHR, —N(R)₂, —N⁺(R)₃, —CX₃, —CN, —OCN, —SCN, —NCO, —NCS, —NO, —NO₂, —N₃, —NC(═O)H, —NC(═O)R, —C(═O)H, —C(═O)R, —C(═O)NH₂, —C(═O)N(R)₂, —SO₃—, —SO₃H, —S(═O)₂R, —OS(═O)₂OR, —S(═O)₂NH₂, —S(═O)₂N(R)₂, —S(═O)R, —OP(═O)(OH)₂, —OP(═O)(OR)₂, —P(═O)(OR)₂, —PO₃, —PO₃H₂, —C(═O)X, —C(═S)R, —CO₂H, —CO₂R, —CO₂—, —C(═S)OR, —C(═O)SR, —C(═S)SR, —C(═O)NH₂,—C(═O)N(R)₂, —C(═S)NH₂, —C(═S)N(R)₂, —C(═NH)NH₂, and —C(═NR)N(R)₂; wherein each X is independently selected for each occasion from F, Cl, Br, and I; and each R is independently selected for each occasion from alkyl, aryl, heterocycloalkyl or heteroaryl, protecting group and prodrug moiety. Wherever a group is described as “optionally substituted,” that group can be substituted with one or more of the above substituents, independently for each occasion. The substitution may include situations in which neighboring substituents have undergone ring closure, such as ring closure of vicinal functional substituents, to form, for instance, lactams, lactones, cyclic anhydrides, acetals, hemiacetals, thioacetals, aminals, and hemiaminals, formed by ring closure, for example, to furnish a protecting group.

It is to be understood that certain radical naming conventions can include either a mono-radical or a di-radical, depending on the context. For example, where a substituent requires two points of attachment to the rest of the molecule, it is understood that the substituent is a di-radical. For example, a substituent identified as alkyl that requires two points of attachment includes di-radicals such as —CH₂—, —CH₂CH₂—, —CH₂CH(CH₃)CH₂—, and the like. Other radical naming conventions clearly indicate that the radical is a di-radical such as “alkylene,” “alkenylene,” “arylene,” “heterocycloalkylene,” and the like.

Wherever a substituent is depicted as a di-radical (i.e., has two points of attachment to the rest of the molecule), it is to be understood that the substituent can be attached in any directional configuration unless otherwise indicated.

### DETAILED DESCRIPTION

The invention provides methods of preventing and/or treating graft-vs-host-disease (GVHD) and autoimmune diseases by administration of an antibody, antigen-binding fragment thereof, ADC, or soluble ligand capable of binding an antigen expressed by hematopoietic cells. In certain embodiments, the methods and compositions disclosed herein may be used to prevent or treat allograft rejection, including host versus graft disease (HvGD). Administration of anti-CD134 or CD278 antibodies or ADCs can cause the selective depletion of a population of exogenous T cells that are reactive against the host following an allogeneic transplant, such as allogeneic bone marrow transplant. The invention is based in part on the discovery that an antibody, antigen-binding fragment thereof, ADC, or soluble ligand, capable of binding CD134 or CD278 can be administered to a patient in in order to prevent and treat GVHD and autoimmune diseases, such as those arising from hematopoietic stem cell transplant therapy, where the anti-CD134 or CD278 agent targets and destroys immune cells, particularly alloreactive T cells, such that the transplant is accepted by the patient. The methods and compositions described herein are beneficial in that general immunosuppressive drugs are not needed, such that the immune system of the patient remains generally intact while specifically targeting cells responsible at least in part for the rejection.

Prevention and treatment of GVHD, due to the administration of anti-CD134 or anti-CD278 antibodies, antigen-binding fragments thereof, ADCs, or soluble ligands can manifest in a variety of clinical symptoms (see, e.g., McDonald, Blood. 127:1544-1440, 2016, and Flowers et al., Blood. 125:606-615, the disclosure of which is incorporated herein by reference as it pertains, but is not limited to, the measureable clinical features of acute and chronic GVHD, respectively). Prevention and treatment of GVHD and autoimmune diseases, due to the administration of anti-CD134 or anti-CD278 antibodies, antigen-binding fragments thereof, or ADCs, can manifest in a variety of empirical measurements. For instance, depletion of CD134+ or CD278+ positive cells can be determined by fluorescence activated cell sorting (FACS) analysis methods known in the art to measure CD134+ or CD278+ white blood cell counts, respectively, in peripheral blood during a post-transplant period, and/or by measuring recovery of marrow cells by donor cells in a bone marrow aspirate sample. Enumeration of the interferon-γ (IFN-γ)-producing T cells in the peripheral blood of recipients can assess the efficacy of anti-CD134 or anti-CD278 against GVHD and autoimmune diseases. The alteration of immune cell populations, as determined by FACS, can be indicative of GVHD or an autoimmune disease. Finally, genetic and proteomic biomarkers taken from the patient can also indicate GVHD or an autoimmune disease.

The sections that follow provide a description of antibodies, antigen-binding fragments thereof, ADCs, or soluble ligands that can be administered to a patient suffering from or at risk for GVHD or an autoimmune disease as well as methods of administering such therapeutics to the patient.

### Anti-CD134 Antibodies and Ligands

The present invention is based in part on the discovery that antibodies, antigen-binding fragments thereof, antibody-drug conjugates (ADC), or soluble ligands capable of binding CD134 (also referred to as OX40, OX40R, or Tumor necrosis factor receptor superfamily member 4 (TNFRSF4)) can be used as therapeutic agents to prevent and treat GVHD from hematopoietic stem cells in a patient suffering from or at risk for GVHD or an autoimmune disease. Additionally, it has been discovered that ligands that bind CD134, such as human CD134L, can be used as a therapeutic agent to prevent or treat patient suffering from or at risk for GVHD. These ligands, such as soluble human CD134, can be covalently bound to an effector domain, such as an Fc domain, for instance, in order to promote antibody-dependent cell-mediated cytotoxicity (ADCC).

T cells have been shown to express CD134, as this antigen is a transmembrane TNF receptor superfamily of costimulatory molecules and is expressed on a variety of hematopoietic cells and promotes T cell activation and regulates proliferation and survival of T cells (see, e.g., Cannons et al., J. Immunol. 167:1313-1324, 2001, the disclosure of which is incorporated herein by reference as it pertains to the expression of CD134 by T cells). Antibodies, antigen-binding fragments thereof, and ligands can be identified using techniques known in the art and described herein, such as by immunization, computational modeling techniques, and in vitro selection methods, such as the phage display and cell-based display platforms described below.

In one embodiment, an anti-CD134 antibody that may be used in the methods and compositions (including ADCs) described herein is the murine monoclonal anti-CD134 antibody Ber-ACT35 or an anti-CD134 antibody comprising antigen binding regions corresponding to the Ber-ACT35 antibody. Ber-ACT35 (sold by Biolegend Cat. No. 350004; see also Santa Cruz Biotechnology, Inc. Cat. No. sc-20073 (date Jan.18, 2019)). Ox40 (BER-Act35) is a mouse monoclonal antibody raised against HuT 102 T cells.

In one embodiment, an anti-CD134 antibody comprises a heavy chain comprising a CDR1, CDR2 and CDR3 of anti-CD134 antibody ACT35, and a light chain variable region comprising a CDR1, CDR2 and CDR3 of anti-CD134 antibody Ber-ACT35. In another embodiment, an anti-CD134 antibody used in the compositions and methods disclosed herein is a humanized Ber-ACT35 antibody.

In one embodiment, an anti-CD134 antibody that may be used in the methods and compositions (including ADCs) described herein is the murine monoclonal anti-CD134 antibody 7D6 or an anti-CD134 antibody comprising antigen binding regions corresponding to the 7D6 antibody. 7D6 (sold by Thermo Fisher Scientific Cat. No. MA5-16548 (dated Jan. 17, 2019); see also Bio Rad, Inc. Cat. No. MCA2568GA (date Jan.18, 2019)). 7D6 is a mouse monoclonal antibody raised against CHO-derived feline CD134-Fc fusion protein.

In one embodiment, an anti-CD134 antibody comprises a heavy chain comprising a CDR1, CDR2 and CDR3 of anti-CD134 antibody 7D6, and a light chain variable region comprising a CDR1, CDR2 and CDR3 of anti-CD134 antibody 7D6. In another embodiment, an anti-CD134 antibody used in the compositions and methods disclosed herein is a humanized 7D6 antibody.

In one embodiment, an anti-CD134 antibody that may be used in the methods and compositions (including ADCs) described herein is the rat monoclonal anti-CD134 antibody 443318 or an anti-CD134 antibody comprising antigen binding regions corresponding to the 443318 antibody. 443318 (sold by Novus Cat. No. MAB3388-SP (dated Jan. 17, 2019); see also Thermo Fisher Scientific. Cat. No. MA5-23676 (date Jan.18, 2019)). 44318 is a rat monoclonal antibody (IgG2A) raised against mouse myeloma cell line NSO-derived recombinant human OX40/TNFRSF4 Leu29-Ala216 (accession number P43489).

In one embodiment, an anti-CD134 antibody comprises a heavy chain comprising a CDR1, CDR2 and CDR3 of anti-CD134 antibody 443318, and a light chain variable region comprising a CDR1, CDR2 and CDR3 of anti-CD134 antibody 443318. In another embodiment, an anti-CD134 antibody used in the compositions and methods disclosed herein is a humanized 443318 antibody.

In other embodiments, additional anti-CD134 antibodies, and antigen-binding fragments thereof, that may be used in conjunction with the methods and compositions (including ADCs) described herein include the following: MEDI6469 (AgonOx, Medimmune), PF-04518600 (Pfizer), vonlerizumab (also known as pogalizumab, MOXR0916, RG7888; Genentech), KHK4083 (Kyowa Hakko Kirin Co., Ltd., Kirin Pharma), BMS 986178 (Bristol-Myers Squibb, Pfizer), tavolimab (also known as MEDI0562, MEDI-0562, tavolixizumab; Medimmune), INCAGN1949 (also known as INCAGN01949; Agenus Inc, Incyte), GBR 830 (also known as VH6/VL9; Glenmark), ATOR-1015 (also known as ADC-1015; Alligator Bioscience), GSK3174998 (GlaxoSmithKline/MD Anderson Cancer Center), MED16383 (Medimmune), MED11109 (Medimmune), IBI101 (Innovent Biologics), UCB patent anti-OX40 (UCB S.A.), U. Texas patent anti-OX40 (also known as Hu222; University of Texas), Crucell patent anti-OX40 (Crucell), Janssen patent anti-OX40 (Bioceros B. V., Janssen Biotech Inc), Glaxo patent anti-OX40 (GlaxoSmithKline, Merck & Co., Inc.), Spring Bioscience patent anti-OX40 (Roche (F. Hoffmann-La Roche Ltd), Spring Bioscience Corp.), Roche patent anti-OX40/FAP (Roche (F. Hoffmann-La Roche Ltd)), DingFu Biotarget patent anti-OX40 (DingFu Biotarget Co. Ltd.), Cancer Research Tech patent anti-OX40 (Cancer Research Technology), Agenus patent anti-GITR/OX40 (Agenus Inc, Ludwig Institute for Cancer Research, Sloan-Kettering Inst. for Cancer Res.), Inhibrx patent anti-PD-L1/OX40 (Inhibrx LLC), Alligator patent anti-OX40/X (Alligator Bioscience), IGM Bio patent anti-OX40 (IGM Biosciences), Sorrento patent anti-OX40 (Sorrento Therapeutics), AbbVie patent anti-OX40 (AbbVie, Inc.), Roche patent anti-OX40/Tenascin C (Roche (F. Hoffmann-La Roche Ltd)), Roche patent anti-OX40/EpCAM (Roche (F. Hoffmann-La Roche Ltd)), and Alligator patent anti-OX40/CTLA-4 (Alligator Bioscience).

Anti-CD134 antibodies that can be used in the methods and compositions disclosed herein, including in conjunction with a cytotoxin described herein, can be identified using techniques known in the art (e.g., hybridoma production or phage display). Hybridomas can be prepared using a murine system. Protocols for immunization and subsequent isolation of splenocytes for fusion are known in the art. Fusion partners and procedures for hybridoma generation are also known. Human anti-CD134 antibodies can also be generated in the HuMAb-Mouse® or XenoMouse™. In making anti-CD134 antibodies, the CD134 antigen is isolated and/or purified. The CD134 antigen may be a fragment of CD134 from the extracellular domain of CD134. Immunization of animals can be performed by any method known in the art. See, e.g., Harlow and Lane, Antibodies: A Laboratory Manual, New York: Cold Spring Harbor Press, 1990. Methods for immunizing animals such as mice, rats, sheep, goats, pigs, cattle and horses are well known in the art. See, e.g., Harlow and Lane, supra, and U.S. Pat. No. 5,994,619. The CD134 antigen may be administered with an adjuvant to stimulate the immune response. Adjuvants known in the art include complete or incomplete Freund's adjuvant, RIBI (muramyl dipeptides) or ISCOM (immunostimulating complexes). After immunization of an animal with a CD134 antigen, antibody-producing immortalized cell lines are prepared from cells isolated from the immunized animal. After immunization, the animal is sacrificed and lymph node and/or splenic B cells are immortalized by methods known in the art (e.g., oncogene transfer, oncogenic virus transduction, exposure to carcinogenic or mutating compounds, fusion with an immortalized cell, e.g., a myeloma cell, and inactivating a tumor suppressor gene. See, e.g., Harlow and Lane, supra. Hybridomas can be selected, cloned and further screened for desirable characteristics, including robust growth, high antibody production and desirable antibody characteristics.

Anti-CD134 antibodies can be generated from an isolated nucleic acid molecule that comprises a nucleotide sequence encoding an amino acid sequence of a CD134 binding molecule provided by the present disclosure. The amino acid sequence encoded by the nucleotide sequence may be any portion of an antibody, such as a CDR, a sequence comprising one, two, or three CDRs, a variable region of a heavy chain, variable region of a light chain, or may be a full-length heavy chain or full length light chain. A nucleic acid of the disclosure can be, for example, DNA or RNA, and may or may not contain intronic sequences. Typically, the nucleic acid is a cDNA molecule.

In addition to antibodies, and antigen-binding fragments, soluble CD134 ligands, such as human CD134 ligand, can be administered to a patient according to the methods described herein to condition a patient prior to hematopoietic stem cell transplant therapy. For instance, CD134 ligands, such as human CD134 ligand, can be conjugated to a cytotoxin (e.g., according to the methods described below or known in the art) or another effector molecule, such as an Fc domain. Maytansine cytotoxins for use with the methods described herein include, for example, human CD134 ligand-IgG1 Fc conjugates, human CD134 ligand-IgG2 Fc conjugates, human CD134 ligand-IgG3 Fc conjugates, human CD134 ligand-IgG4 Fc conjugates, human CD134 ligand-IgA Fc conjugates, human CD134 ligand-IgE Fc conjugates, human CD134 ligand-IgM Fc conjugates, and human CD134 ligand-IgD Fc conjugates.

Antibodies and ligands for use in conjunction with the compositions and methods described herein include variants of those antibodies described above, such as antibody fragments that contain or lack an Fc domain, as well as humanized variants of non-human antibodies described herein and antibody-like protein scaffolds (e.g., ¹⁰Fn3 domains) containing one or more, or all, of the CDRs or equivalent regions thereof of an antibody, antibody fragment, or soluble ligand described herein. Exemplary antigen-binding fragments of the foregoing antibodies include a dual-variable immunoglobulin domain, a single-chain Fv molecule (scFv), a diabody, a triabody, a nanobody, an antibody-like protein scaffold, a Fv fragment, a Fab fragment, a F(ab′)₂ molecule, and a tandem di-scFv, among others.

The antibodies of the invention may be engineered to further modulate antibody half-life by introducing additional Fc mutations, such as those described for example in (Dall'Acqua et al. (2006) J Biol Chem 281: 23514-24), (Zalevsky et al. (2010) Nat Biotechnol 28: 157-9), (Hinton et al. (2004) J Biol Chem 279: 6213-6), (Hinton et al. (2006) J Immunol 176: 346-56), (Shields et al. (2001) J Biol Chem 276: 6591-604), (Petkova et al. (2006) Int Immunol 18: 1759-69), (Datta-Mannan et al. (2007) Drug Metab Dispos 35: 86-94), (Vaccaro et al. (2005) Nat Biotechnol 23: 1283-8), (Yeung et al. (2010) Cancer Res 70: 3269-77) and (Kim et al. (1999) Eur J Immunol 29: 2819-25), and include positions 250, 252, 253, 254, 256, 257, 307, 376, 380, 428, 434 and 435. Exemplary mutations that may be made singularly or in combination are T250Q, M252Y, 1253A, S254T, T256E, P2571, T307A, D376V, E380A, M428L, H433K, N434S, N434A, N434H, N434F, H435A and H435R mutations.

The foregoing anti-CD134 antibodies, or antigen-binding fragments thereof, can be used in various aspects of the invention set forth herein, including, for example, in methods for depletion of CD134+ cells in a human subject. The foregoing anti-CD134 antibodies, or antigen-binding fragments thereof, can also be conjugated to an agent, e.g., a cytotoxin, for example, an amatoxin, as described herein.

### Anti-CD278 Antibodies

The present invention is further based in part on the discovery that antibodies, antigen-binding fragments thereof, antibody-drug conjugates (ADC), or soluble ligands capable of binding CD278 (also referred to as ICOS, AILIM, activation inducible lymphocyte immunomodulatory molecule) can be used as therapeutic agents to prevent and treat GVHD from hematopoietic stem cells in a patient suffering from or at risk for GVHD or an autoimmune disease.

CD278 or ICOS (Inducible T-cell COStimulator) is a CD28-superfamily costimulatory molecule that is expressed on activated T cells. CD278 belongs to the CD28 and CTLA-4 cell-surface receptor family, and plays an important role in cell-cell signaling, immune responses, and regulation of cell proliferation.

In one embodiment, an anti-CD278 antibody comprises a heavy chain comprising a CDR1, CDR2 and CDR3 of anti-CD278 antibody DX29, and a light chain variable region comprising a CDR1, CDR2 and CDR3 of anti-CD134 antibody DX29. In another embodiment, an anti-CD278 antibody used in the compositions and methods disclosed herein is a humanized DX29 antibody.

In one embodiment, an anti-CD278 antibody that may be used in the methods and compositions (including ADCs) described herein is the murine monoclonal anti-CD278 antibody DX29 or an anti-CD278 antibody comprising antigen binding regions corresponding to the DX29 antibody. DX29 (sold by BD Biosciences Cat. No. 557801(dated Jan. 17, 2019); see also Fisher Scientific. Cat. No. BDB557802 (date Jan.18, 2019)). DX29 is a mouse monoclonal antibody raised against activated human T cells.

In one embodiment, an anti-CD278 antibody comprises a heavy chain comprising a CDR1, CDR2 and CDR3 of anti-CD278 antibody 669238, and a light chain variable region comprising a CDR1, CDR2 and CDR3 of anti-CD134 antibody 669238. In another embodiment, an anti-CD278 antibody used in the compositions and methods disclosed herein is a humanized 669238 antibody.

In one embodiment, an anti-CD278 antibody that may be used in the methods and compositions (including ADCs) described herein is the murine monoclonal anti-CD278 antibody DX29 or an anti-CD278 antibody comprising antigen binding regions corresponding to the 669238 antibody. 669238 (sold by Novus Cat. No. MAB69751-SP; see also Fisher Scientific Cat. No. MAB69752 (date Jan.18, 2019)). 669238 is a mouse monoclonal antibody raised against partial recombinant human ICOS protein (amino acids 21-141) [UniProt Q9Y6W8].

In other embodiments, additional anti-CD278 antibodies, and antigen-binding fragments thereof, that may be used in conjunction with the methods and compositions (including ADCs) described herein include the following: MEDI-570 (also known as JMab-136; Medimmune), GSK3359609 (also known as 88-2, 53-3, 92-17, IgG4PE; GlaxoSmithKline, INSERM), vopratelimab (also known as JTX-2011; Jounce Therapeutics), XmAb23104 (Xencor Inc.), KY1044 (Kymab Ltd.), Japan Tobacco patent anti-ICOS (Japan Tobacco Inc), Kymab patent anti-ICOS (Kymab Ltd.), and BMS patent anti-ICOS (Bristol-Myers Squibb).

Anti-CD278 antibodies that can be used in conjunction with a cytotoxin described herein can be identified using techniques known in the art (e.g., hybridoma production). Hybridomas can be prepared using a murine system. Protocols for immunization and subsequent isolation of splenocytes for fusion are known in the art. Fusion partners and procedures for hybridoma generation are also known. Human anti-CD278 antibodies can also be generated in the HuMAb-Mouse® or XenoMouse™. In making anti-CD278 antibodies, the CD278 antigen is isolated and/or purified. The CD278 antigen may be a fragment of CD278 from the extracellular domain of CD278. Immunization of animals can be performed by any method known in the art. See, e.g., Harlow and Lane, Antibodies: A Laboratory Manual, New York: Cold Spring Harbor Press, 1990. Methods for immunizing animals such as mice, rats, sheep, goats, pigs, cattle and horses are well known in the art. See, e.g., Harlow and Lane, supra, and U.S. Pat. No. 5,994,619. The CD278 antigen may be administered with an adjuvant to stimulate the immune response. Adjuvants known in the art include complete or incomplete Freund's adjuvant, RIBI (muramyl dipeptides) or ISCOM (immunostimulating complexes). After immunization of an animal with a CD278 antigen, antibody-producing immortalized cell lines are prepared from cells isolated from the immunized animal. After immunization, the animal is sacrificed and lymph node and/or splenic B cells are immortalized by methods known in the art (e.g., oncogene transfer, oncogenic virus transduction, exposure to carcinogenic or mutating compounds, fusion with an immortalized cell, e.g., a myeloma cell, and inactivating a tumor suppressor gene. See, e.g., Harlow and Lane, supra. Hybridomas can be selected, cloned and further screened for desirable characteristics, including robust growth, high antibody production and desirable antibody characteristics.

Anti-CD278 antibodies can be generated from an isolated nucleic acid molecule that comprises a nucleotide sequence encoding an amino acid sequence of a CD278 binding molecule provided by the present disclosure. The amino acid sequence encoded by the nucleotide sequence may be any portion of an antibody, such as a CDR, a sequence comprising one, two, or three CDRs, a variable region of a heavy chain, variable region of a light chain, or may be a full-length heavy chain or full length light chain. A nucleic acid of the disclosure can be, for example, DNA or RNA, and may or may not contain intronic sequences. Typically, the nucleic acid is a cDNA molecule.

In addition to antibodies, and antigen-binding fragments, soluble CD278 ligands, such as human CD278 ligand, can be administered to a patient according to the methods described herein to prevent allograft rejection, e.g., following hematopoietic stem cell transplant therapy. For instance, CD278 ligands, such as human CD278 ligand, can be conjugated to a cytotoxin (e.g., according to the methods described below or known in the art) or another effector molecule, such as an Fc domain. Maytansine cytotoxins for use with the methods described herein include, for example, human CD278 ligand-IgG1 Fc conjugates, human CD278 ligand-IgG2 Fc conjugates, human CD278 ligand-IgG3 Fc conjugates, human CD278 ligand-IgG4 Fc conjugates, human CD278 ligand-IgA Fc conjugates, human CD278 ligand-IgE Fc conjugates, human CD278 ligand-IgM Fc conjugates, and human CD278 ligand-IgD Fc conjugates.

Antibodies and ligands for use in conjunction with the compositions and methods described herein include variants of those antibodies described above, such as antibody fragments that contain or lack an Fc domain, as well as humanized variants of non-human antibodies described herein and antibody-like protein scaffolds (e.g., ¹⁰Fn3 domains) containing one or more, or all, of the CDRs or equivalent regions thereof of an antibody, antibody fragment, or soluble ligand described herein. Exemplary antigen-binding fragments of the foregoing antibodies include a dual-variable immunoglobulin domain, a single-chain Fv molecule (scFv), a diabody, a triabody, a nanobody, an antibody-like protein scaffold, a Fv fragment, a Fab fragment, a F(ab′)₂ molecule, and a tandem di-scFv, among others.

The antibodies of the invention may be engineered to further modulate antibody half-life by introducing additional Fc mutations, such as those described for example in (Dall'Acqua et al. (2006) J Biol Chem 281: 23514-24), (Zalevsky et al. (2010) Nat Biotechnol 28: 157-9), (Hinton et al. (2004) J Biol Chem 279: 6213-6), (Hinton et al. (2006) J Immunol 176: 346-56), (Shields et al. (2001) J Biol Chem 276: 6591-604), (Petkova et al. (2006) Int Immunol 18: 1759-69), (Datta-Mannan et al. (2007) Drug Metab Dispos 35: 86-94), (Vaccaro et al. (2005) Nat Biotechnol 23: 1283-8), (Yeung et al. (2010) Cancer Res 70: 3269-77) and (Kim et al. (1999) Eur J Immunol 29: 2819-25), and include positions 250, 252, 253, 254, 256, 257, 307, 376, 380, 428, 434 and 435. Exemplary mutations that may be made singularly or in combination are T250Q, M252Y, 1253A, S254T, T256E, P2571, T307A, D376V, E380A, M428L, H433K, N434S, N434A, N434H, N434F, H435A and H435R mutations.

The foregoing anti-CD278 antibodies, or antigen-binding fragments thereof, can be used in various aspects of the invention set forth herein, including, for example, in methods for depletion of CD278+ cells in a human subject. The foregoing anti-CD278 antibodies, or antigen-binding fragments thereof, can also be conjugated to an agent, e.g., a cytotoxin, for example, an amatoxin, as described herein.

The anti-CD134 or CD278 antibodies or binding fragments described herein may also include modifications and/or mutations that alter the properties of the antibodies and/or fragments, such as those that increase half-life, increase or decrease ADCC, etc., as is known in the art.

In one embodiment, the anti-CD134 or anti-CD278 antibody, or binding fragment thereof, comprises a variant Fc region, wherein said variant Fc region comprises at least one amino acid modification relative to a wild-type Fc region, such that said molecule has an altered affinity for an FcgammaR. Certain amino acid positions within the Fc region are known through crystallography studies to make a direct contact with FcγR. Specifically amino acids 234-239 (hinge region), amino acids 265-269 (B/C loop), amino acids 297-299 (C′/E loop), and amino acids 327-332 (F/G) loop. (see Sondermann et al., 2000 Nature, 406: 267-273). Thus, the anti-CD134 or anti-CD278 antibodies described herein may comprise variant Fc regions comprising modification of at least one residue that makes a direct contact with an Fcγ R based on structural and crystallographic analysis. In one embodiment, the Fc region of the anti-CD134 or anti-CD278 antibody (or fragment thereof) comprises an amino acid substitution at amino acid 265 according to the EU index as in Kabat et al., Sequences of Proteins of Immunological Interest, 5th Ed. Public Health Service, NH1, MD (1991), expressly incorporated herein by references. The “EU index as in Kabat” refers to the numbering of the human IgG1 EU antibody. The EU index or EU index as in Kabat or EU numbering scheme refers to the numbering of the EU antibody (Edelman et al., 1969, Proc Natl Acad Sci USA 63:78-85, hereby entirely incorporated by reference.) In one embodiment, the Fc region comprises a D265A mutation. In one embodiment, the Fc region comprises a D265C mutation. In some embodiments, the Fc region of the anti-CD134 or anti-CD278 antibody (or fragment thereof) comprises an amino acid substitution at amino acid 234 according to the EU index as in Kabat. In one embodiment, the Fc region comprises a L234A mutation. In some embodiments, the Fc region of the anti-CD134 or anti-CD278 antibody (or fragment thereof) comprises an amino acid substitution at amino acid 235 according to the EU index as in Kabat. In one embodiment, the Fc region comprises a L235A mutation. In yet another embodiment, the Fc region comprises a L234A and L235A mutation . In a further embodiment, the Fc region comprises a D265C, L234A, and L235A mutation.

In certain aspects a variant IgG Fc domain comprises one or more amino acid substitutions resulting in decreased or ablated binding affinity for an Fc.gamma.R and/or C1q as compared to the wild type Fc domain not comprising the one or more amino acid substitutions. Fc binding interactions are essential for a variety of effector functions and downstream signaling events including, but not limited to, antibody dependent cell-mediated cytotoxicity (ADCC) and complement dependent cytotoxicity (CDC). Accordingly, in certain aspects, an antibody comprising a modified Fc region (e.g., comprising a L234A, L235A, and a D265C mutation) has substantially reduced or abolished effector functions.

Affinity to an Fc region can be determined using a variety of techniques known in the art, for example but not limited to, equilibrium methods (e.g., enzyme-linked immunoabsorbent assay (ELISA); KinExA, Rathanaswami et al. Analytical Biochemistry, Vol. 373:52-60, 2008; or radioimmunoassay (RIA)), or by a surface plasmon resonance assay or other mechanism of kinetics-based assay (e.g., BIACORE™. analysis or Octet™ analysis (forteBIO)), and other methods such as indirect binding assays, competitive binding assays fluorescence resonance energy transfer (FRET), gel electrophoresis and chromatography (e.g., gel filtration). These and other methods may utilize a label on one or more of the components being examined and/or employ a variety of detection methods including but not limited to chromogenic, fluorescent, luminescent, or isotopic labels. A detailed description of binding affinities and kinetics can be found in Paul, W. E., ed., Fundamental Immunology, 4th Ed., Lippincott-Raven, Philadelphia (1999), which focuses on antibody-immunogen interactions. One example of a competitive binding assay is a radioimmunoassay comprising the incubation of labeled antigen with the antibody of interest in the presence of increasing amounts of unlabeled antigen, and the detection of the antibody bound to the labeled antigen. The affinity of the antibody of interest for a particular antigen and the binding off-rates can be determined from the data by scatchard plot analysis. Competition with a second antibody can also be determined using radioimmunoassays. In this case, the antigen is incubated with antibody of interest conjugated to a labeled compound in the presence of increasing amounts of an unlabeled second antibody.

The antibodies described herein may be further engineered to further modulate antibody half-life by introducing additional Fc mutations, such as those described for example in (Dall'Acqua et al. (2006) J Biol Chem 281: 23514-24), (Zalevsky et al. (2010) Nat Biotechnol 28: 157-9), (Hinton et al. (2004) J Biol Chem 279: 6213-6), (Hinton et al. (2006) J Immunol 176: 346-56), (Shields et al. (2001) J Biol Chem 276: 6591-604), (Petkova et al. (2006) Int Immunol 18: 1759-69), (Datta-Mannan et al. (2007) Drug Metab Dispos 35: 86-94), (Vaccaro et al. (2005) Nat Biotechnol 23: 1283-8), (Yeung et al. (2010) Cancer Res 70: 3269-77) and (Kim et al. (1999) Eur J Immunol 29: 2819-25), and include positions 250, 252, 253, 254, 256, 257, 307, 376, 380, 428, 434 and 435. Exemplary mutations that may be made singularly or in combination are T250Q, M252Y, 1253A, S254T, T256E, P2571, T307A, D376V, E380A, M428L, H433K, N434S, N434A, N434H, N434F, H435A and H435R mutations.

Thus, in one embodiment, the Fc region comprises a mutation resulting in a decrease in half life. An antibody having a short half life may be advantageous in certain instances where the antibody is expected to function as a short-lived therapeutic. In one embodiment, the Fc regions comprise a mutation at position 435 (EU index according to Kabat). In one embodiment, the mutation is an H435A mutation.

In one embodiment, the anti-CD134 or anti-CD278 antibody described herein has a half life of equal to or less than 24 hours, a half life of equal to or less than 22 hours, a half life of equal to or less than 20 hours, a half life of equal to or less than 18 hours, a half life of equal to or less than 16 hours, a half life of equal to or less than 14 hours, equal to or less than 13 hours, equal to or less than 12 hours, or equal to or less than 11 hours. In one embodiment, the half life of the antibody is 11 hours to 24 hours; 12 hours to 22 hours; 10 hours to 20 hours; 8 hours to 18 hours; or 14 hours to 24 hours.

In some aspects, the Fc region comprises two or more mutations that confer reduced half-life and greatly diminish or completely abolish an effector function of the antibody. In some embodiments, the Fc region comprises a mutation resulting in a decrease in half-life and a mutation of at least one residue that can make direct contact with an FcγR (e.g., as based on structural and crystallographic analysis). In one embodiment, the Fc region comprises a H435A mutation, a L234A mutation, and a L235A mutation. In one embodiment, the Fc region comprises a H435A mutation and a D265C mutation. In one embodiment, the Fc region comprises a H435A mutation, a L234A mutation, a L235A mutation, and a D265C mutation.

In some embodiments, the antibody or antigen-binding fragment thereof is conjugated to a cytotoxin (e.g., amatoxin) by way of a cysteine residue in the Fc domain of the antibody or antigen-binding fragment thereof. In some embodiments, the cysteine residue is introduced by way of a mutation in the Fc domain of the antibody or antigen-binding fragment thereof. For instance, the cysteine residue may be selected from the group consisting of Cys118, Cys239, and Cys265. In one embodiment, the Fc region of the anti-CD134 or anti-CD278 antibody (or fragment thereof) comprises an amino acid substitution at amino acid 265 according to the EU index as in Kabat. In one embodiment, the Fc region comprises a D265C mutation. In one embodiment, the Fc region comprises a D265C and H435A mutation. In one embodiment, the Fc region comprises a D265C, a L234A, and a L235A mutation. In one embodiment, the Fc region comprises a D265C, a L234A, a L235A, and a H435A mutation.

In some embodiments of these aspects, the cysteine residue is naturally occurring in the Fc domain of the antibody or antigen-binding fragment thereof. For instance, the Fc domain may be an IgG Fc domain, such as a human IgG1 Fc domain, and the cysteine residue may be selected from the group consisting of Cys261, Csy321, Cys367, and Cys425.

The variant Fc domains described herein are defined according to the amino acid modifications that compose them. For all amino acid substitutions discussed herein in regard to the Fc region, numbering is always according to the EU index. Thus, for example, D265C is an Fc variant with the aspartic acid (D) at EU position 265 substituted with cysteine (C) relative to the parent Fc domain. Likewise, e.g., D265C/L234A/L235A defines a variant Fc variant with substitutions at EU positions 265 (D to C), 234 (L to A), and 235 (L to A) relative to the parent Fc domain. A variant can also be designated according to its final amino acid composition in the mutated EU amino acid positions. For example, the L234A/L235A mutant can be referred to as LALA. It is noted that the order in which substitutions are provided is arbitrary.

### Methods of Identifying Antibodies

Methods for high throughput screening of libraries of antibodies, antibody fragments, and ligands for molecules capable of binding CD134 or CD278 can be used to identify and affinity mature agents that are, for example, useful for preventing and treating GVHD or autoimmune diseases. Such methods include in vitro display techniques known in the art, such as phage display, bacterial display, yeast display, mammalian cell display, ribosome display, mRNA display, and cDNA display, among others. The use of phage display to isolate antibodies, antigen-binding fragments, or ligands that bind biologically relevant molecules has been reviewed, for example, in Felici et al., Biotechnol. Annual Rev. 1:149-183, 1995; Katz, Annual Rev. Biophys. Biomol. Struct. 26:27-45, 1997; and Hoogenboom et al., Immunotechnology 4:1-20, 1998, the disclosures of each of which are incorporated herein by reference as they pertain to in vitro display techniques. Randomized combinatorial peptide libraries have been constructed to select for polypeptides that bind cell surface antigens as described in Kay, Perspect. Drug Discovery Des. 2:251-268, 1995 and Kay et al., Mol. Divers. 1:139-140, 1996, the disclosures of each of which are incorporated herein by reference as they pertain to the discovery of antigen-binding molecules. Proteins, such as multimeric proteins, have been successfully phage-displayed as functional molecules (see, for example, EP 0349578; EP 4527839; and EP 0589877, as well as Chiswell and McCafferty, Trends Biotechnol. 10:80-84 1992, the disclosures of each of which are incorporated herein by reference as they pertain to the use of in vitro display techniques for the discovery of antigen-binding molecules. In addition, functional antibody fragments, such as Fab and scFv fragments, have been expressed in in vitro display formats (see, for example, McCafferty et al., Nature 348:552-554, 1990; Barbas et al., Proc. Natl. Acad. Sci. USA 88:7978-7982, 1991; and Clackson et al., Nature 352:624-628, 1991, the disclosures of each of which are incorporated herein by reference as they pertain to in vitro display platforms for the discovery of antigen-binding molecules). Human anti-CD134 antibodies or human anti-CD278 antibodies can also be generated, for example, in the HuMAb-Mouse® or XenoMouse™. These techniques, among others, can be used to identify and improve the affinity of antibodies, antibody fragments, and ligands that bind CD134 or CD278 that can in turn be used to deplete hematopoietic cells in a patient.

In addition to in vitro display techniques, computational modeling techniques can be used to design and identify anti-CD134 or anti-CD278 antibodies, antibody fragments and ligands in silico, for instance, using the procedures described in US 2013/0288373, the disclosure of which is incorporated herein as it pertains to molecular modeling methods for identifying anti-CD134 or anti-CD278 antibodies. For example, using computational modeling techniques, one of skill in the art can screen libraries of antibodies, antibody fragments, and ligands in silico for molecules capable of binding specific epitopes on CD134 or CD278, such as extracellular epitopes of CD134 or CD278, respectively.

Additional techniques can be used to identify antibodies, antigen-binding fragments, and ligands thereof that bind CD134 or CD278 on the surface of a cell (e.g., a T cell) and that are internalized by the cell, for instance, by receptor-mediated endocytosis. For example, the in vitro display techniques described above can be adapted to screen for antibodies, antigen-binding fragments thereof, and ligands that bind CD134 or CD278 on the surface of a hematopoietic stem cell and that are subsequently internalized. Phage display represents one such technique that can be used in conjunction with this screening paradigm. To identify anti-CD134 or anti-CD278 antibodies, fragments thereof, and ligands that bind CD134 or CD278 and are subsequently internalized by hematopoietic stem cells, one of skill in the art can use the phage display techniques described in Williams et al., Leukemia 19:1432-1438, 2005, the disclosure of which is incorporated herein by reference in its entirety. For example, using mutagenesis methods known in the art, recombinant phage libraries can be produced that encode antibodies, antibody fragments, such as scFv fragments, Fab fragments, diabodies, triabodies, and ¹⁰Fn3 domains, among others, or ligands that contain randomized amino acid cassettes (e.g., in one or more, or all, of the CDRs or equivalent regions thereof or an antibody or antibody fragment). The framework regions, hinge, Fc domain, and other regions of the antibodies or antibody fragments may be designed such that they are non-immunogenic in humans, for instance, by virtue of having human germline antibody sequences or sequences that exhibit only minor variations relative to human germline antibodies.

Using phage display techniques described herein or known in the art, phage libraries containing randomized antibodies, antibody fragments, or ligands covalently bound to the phage particles can be incubated with CD134 or CD278 antigen, for instance, by first incubating the phage library with blocking agents (such as, for instance, milk protein, bovine serum albumin, and/or IgG so as to remove phage encoding antibodies, fragments thereof, or ligands that exhibit non-specific protein binding and phage that encode antibodies or fragments thereof that bind Fc domains, and then incubating the phage library with a population of hematopoietic stem cells, which are CD134+ or CD278+. The phage library can be incubated with the hematopoietic stem cells for a time sufficient to allow CD134 specific antibodies, antigen-binding fragments thereof, or ligands to bind cell-surface CD134 or CD278 and to subsequently be internalized by the hematopoietic stem cells (e.g., from 30 minutes to 6 hours at 4° C., such as 1 hour at 4° C.). Phage containing antibodies, fragments thereof, or ligands that do not exhibit sufficient affinity for CD134 or CD278 so as to permit binding to, and internalization by, hematopoietic stem cells can subsequently be removed by washing the cells, for instance, with cold (4° C.) 0.1 M glycine buffer at pH 2.8. Phage bound to antibodies, fragments thereof, or ligands that have been internalized by the hematopoietic stem cells can be identified, for instance, by lysing the cells and recovering internalized phage from the cell culture medium. The phage can then be amplified in bacterial cells, for example, by incubating bacterial cells with recovered phage in 2xYT medium using methods known in the art. Phage recovered from this medium can then be characterized, for instance, by determining the nucleic acid sequence of the gene(s) encoding the antibodies, fragments thereof, or ligands inserted within the phage genome. The encoded antibodies, fragments thereof, or ligands can subsequently be prepared de novo by chemical synthesis (for instance, of antibody fragments, such as scFv fragments, or CD134 or CD278 ligands) or by recombinant expression (for instance, of full-length antibodies).

The internalizing capacity of the prepared antibodies, fragments thereof, or ligands can be assessed, for instance, using radionuclide internalization assays known in the art. For example, anti-CD134 or anti-CD278 antibodies, fragments thereof, or ligands identified using in vitro display techniques described herein or known in the art can be functionalized by incorporation of a radioactive isotope, such as ¹⁸F, ⁷⁵Br, ⁷⁷Br, ¹²²I, ¹²³I, ¹²⁴I, ¹²⁵I, ¹²⁹I, ¹³¹I, ²¹¹At, ⁶⁷Ga, ¹¹¹In, ⁹⁹Tc, ¹⁶⁹Yb, ¹⁸⁶Re, ⁶⁴Cu, ⁶⁷Cu, ¹⁷⁷Lu, ⁷⁷As, ⁷²As, ⁸⁶Y, ⁹⁰Y, ⁸⁹Zr, ²¹²Bi, ²¹³Bi, or ²²⁵Ac. For instance, radioactive halogens, such as ¹⁸F, ⁷⁵Br, ⁷⁷Br, ¹²²I, ¹²³I, ¹²⁴I, ¹²⁵I, ¹²⁹I, ¹³¹I, ²¹¹At, can be incorporated into antibodies, fragments thereof, or ligands using beads, such as polystyrene beads, containing electrophilic halogen reagents (e.g., Iodination Beads, Thermo Fisher Scientific, Inc., Cambridge, Mass.). Radiolabeled antibodies, fragments thereof, ADCs, or ligands can be incubated with hematopoietic stem cells for a time sufficient to permit internalization (e.g., from 30 minutes to 6 hours at 4° C., such as 1 hour at 4° C.). The cells can then be washed to remove non-internalized antibodies or fragments thereof, (e.g., using cold (4° C.) 0.1 M glycine buffer at pH 2.8). Internalized antibodies, fragments thereof, or ligands can be identified by detecting the emitted radiation (e.g., γ-radiation) of the resulting hematopoietic stem cells in comparison with the emitted radiation (e.g., γ-radiation) of the recovered wash buffer. The foregoing internalization assays can also be used to characterize ADCs.

Antibodies may be produced using recombinant methods and compositions, e.g., as described in U.S. Pat. No. 4,816,567. In one embodiment, isolated nucleic acid encoding an anti-CD134 or anti-CD278 antibody described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., Y0, NS0, Sp20 cell). In one embodiment, a method of making an anti-CLL-1 antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an anti-CD134 or anti-CD278 antibody, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

An exemplary method for in vitro evolution of anti-CD134 or anti-CD278 antibodies for use with the compositions and methods described herein is phage display. Phage display libraries can be created by making a designed series of mutations or variations within a coding sequence for the CDRs of an antibody or the analogous regions of an antibody-like scaffold (e.g., the BC, CD, and DE loops of ¹⁰Fn3 domains). The template antibody-encoding sequence into which these mutations are introduced may be, for example, a naive human germline sequence. These mutations can be performed using standard mutagenesis techniques known in the art. Each mutant sequence thus encodes an antibody corresponding to the template save for one or more amino acid variations. Retroviral and phage display vectors can be engineered using standard vector construction techniques known in the art. P3 phage display vectors along with compatible protein expression vectors can be used to generate phage display vectors for antibody diversification.

The mutated DNA provides sequence diversity, and each transformant phage displays one variant of the initial template amino acid sequence encoded by the DNA, leading to a phage population (library) displaying a vast number of different but structurally related amino acid sequences. Due to the well-defined structure of antibody hypervariable regions, the amino acid variations introduced in a phage display screen are expected to alter the binding properties of the binding peptide or domain without significantly altering its overall molecular structure.

In a typical screen, a phage library may be contacted with and allowed to bind CD134 or CD278 or an epitope thereof. To facilitate separation of binders and non-binders, it is convenient to immobilize the target on a solid support. Phage bearing a CD134-binding moiety or CD278-binding moiety can form a complex with the target on the solid support, whereas non-binding phage remain in solution and can be washed away with excess buffer. Bound phage can then liberated from the target by changing the buffer to an extreme pH (pH 2 or pH 10), changing the ionic strength of the buffer, adding denaturants, or other known means.

The recovered phage can then be amplified through infection of bacterial cells, and the screening process can be repeated with the new pool that is now depleted in non-binding antibodies and enriched for antibodies that bind CD134 or CD278. The recovery of even a few binding phage is sufficient to amplify the phage for a subsequent iteration of screening. After a few rounds of selection, the gene sequences encoding the antibodies or antigen-binding fragments thereof derived from selected phage clones in the binding pool are determined by conventional methods, thus revealing the peptide sequence that imparts binding affinity of the phage to the target. During the panning process, the sequence diversity of the population diminishes with each round of selection until desirable peptide-binding antibodies remain. The sequences may converge on a small number of related antibodies or antigen-binding fragments thereof. An increase in the number of phage recovered at each round of selection is an indication that convergence of the library has occurred in a screen.

Non-human antibodies that bind CD134 or CD278 can be humanized, for instance, according to the following procedure. Consensus human antibody heavy chain and light chain sequences are known in the art (see e.g., the “VBASE” human germline sequence database; Kabat et al. Sequences of Proteins of Immunological Interest, Fifth Edition, U.S. Department of Health and Human Services, NIH Publication No. 91-3242, 1991; Tomlinson et al., J. Mol. Biol. 227:776-798, 1992; and Cox et al. Eur. J. Immunol. 24:827-836, 1994, the disclosures of each of which are incorporated herein by reference as they pertain to consensus human antibody heavy chain and light chain sequences. Using established procedures, one of skill in the art can identify the variable domain framework residues and CDRs of a consensus antibody sequence (e.g., by sequence alignment). One can substitute one or more CDRs of the heavy chain and/or light chain variable domains of consensus human antibody with one or more corresponding CDRs of a non-human antibody that binds CD134 or CD278 in order to produce a humanized antibody.

To produce humanized antibodies, one can recombinantly express a polynucleotide encoding the above consensus sequence in which one or more variable region CDRs have been replaced with one or more variable region CDR sequences of a non-human antibody that binds CD134 or CD278. As the affinity of the antibody for CD134 or CD278 is determined primarily by the CDR sequences, the resulting humanized antibody is expected to exhibit an affinity for CD134 or CD278 that is about the same as that of the non-human antibody from which the humanized antibody was derived. Methods of determining the affinity of an antibody for a target antigen include, for instance, ELISA-based techniques described herein and known in the art, as well as surface plasmon resonance, fluorescence anisotropy, and isothermal titration calorimetry, among others.

### Antibody-Drug Conjugates (ADCs)

### Cytotoxins

Antibodies, antigen-binding fragments thereof, and ligands described herein (e.g., antibodies, antigen-binding fragments thereof, and soluble ligands that recognize and bind CD134 or CD278) can be conjugated (or linked) to a cytotoxin, such as a microtubule-binding agent (for instance, maytansine or a maytansinoid), an amatoxin, pseudomonas exotoxin A, deBouganin, or diphtheria toxin, such as α-amanitin, saporin, an auristatin, an anthracycline, a calicheamicin, irinotecan, SN-38, a duocarmycin, a pyrrolobenzodiazepine, a pyrrolobenzodiazepine dimer, an indolinobenzodiazepine, and an indolinobenzodiazepine dimer, or a variant thereof, or another cytotoxic compound described herein or known in the art in order to promote the depletion of hematopoietic cells, such as a host-reactive T cell, upon administration to a patient. In some embodiments, the cytotoxic molecule is conjugated to an internalizing anti-CD134 or CD278 antibody, antigen-binding fragment thereof or soluble ligand, such that following the cellular uptake of the antibody, fragment thereof, or soluble ligand, the cytotoxin may access its intracellular target and mediate hematopoietic cell death. Additional cytotoxins suitable for use with the compositions and methods described herein include DNA-intercalating agents, (e.g., anthracyclines), agents capable of disrupting the mitotic spindle apparatus (e.g., vinca alkaloids, maytansine, maytansinoids, and derivatives thereof), RNA polymerase inhibitors (e.g., an amatoxin, such as α-amanitin, and derivatives thereof), agents capable of disrupting protein biosynthesis (e.g., agents that exhibit rRNA N-glycosidase activity, such as saporin and ricin A-chain), among others known in the art.

### Maytansinoids

Anti-CD134 or anti-CD278 antibodies can be conjugated to a cytotoxin that is a microtubule binding agent. In some embodiments, the cytotoxin is a maytansine, maytansinoid or maytansinoid analog. Maytansinoids are microbtubule binding agents that prohibit tubulin polymerization. Examples of suitable maytansinoids include esters of maytansinol, synthetic maytaninol, and maytansinol analogs and derivatives. Included are any drugs that inhibit microtubule formation and that are highly toxic to mammalian cells, as are maytansinoids, maytansinol, and maytansinol analogs, and derivatives.

Examples of suitable maytansinol esters include those having a modified aromatic ring and those having modifications at other positions. Such suitable maytansinoids are disclosed in U.S. Pat. Nos. 4,137,230; 4,151,042; 4,248,870; 4,256,746; 4,260,608; 4,265,814; 4,294,757; 4,307,016; 4,308,268; 4,308,269; 4,309,428; 4,313,946; 4,315,929; 4,317,821; 4,322,348; 4,331,598; 4,361,650; 4,362,663; 4,364,866; 4,424,219; 4,450,254; 4,322,348; 4,362,663; 4,371,533; 5,208,020; 5,416,064; 5,475,092; 5,585,499; 5,846,545; 6,333,410; 7,276,497; and 7,473,796, the disclosures of each of which are incorporated herein by reference as they pertain to maytansinoids and derivatives thereof.

In some embodiments, the immunoconjugates of the invention utilize the thiol-containing maytansinoid (DM1), formally termed N²′-deacetyl-N²′-(3-mercapto-1-oxopropyl)-maytansine, as the cytotoxic agent. DM1 is represented by the following structural formula:

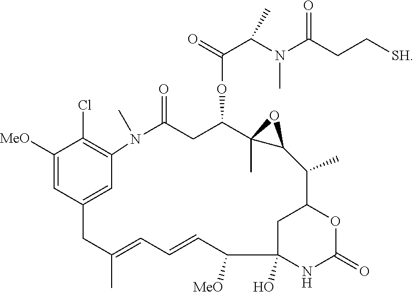 [SMILES: COc1cc2cc(c1Cl)N(C)C(=O)C[C@H](OC(=O)[C@H](C)N(C)C(=O)CCS)[C@]1(C)O[C@H]1[C@H](C)C1C[C@@](O)(NC(=O)O1)[C@H](OC)/C=C/C=C(\C)C2]

In another embodiment, the conjugates of the present invention utilize the thiol-containing maytansinoid N²′-deacetyl-N²′(4-methyl-4-mercapto-1-oxopentyl)-maytansine (e.g., DM4) as the cytotoxic agent. DM4 is represented by the following structural formula:

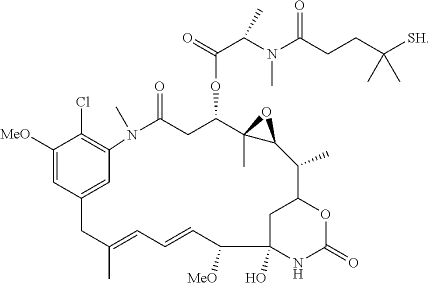 [SMILES: COc1cc2cc(c1Cl)N(C)C(=O)C[C@H](OC(=O)[C@H](C)N(C)C(=O)CCC(C)(C)S)[C@]1(C)O[C@H]1[C@H](C)C1C[C@@](O)(NC(=O)O1)[C@H](OC)/C=C/C=C(\C)C2]

Another maytansinoid comprising a side chain that contains a sterically hindered thiol bond is N²′-deacetyl-N-²′(4-mercapto-1-oxopentyl)-maytansine (termed DM3), represented by the following structural formula:

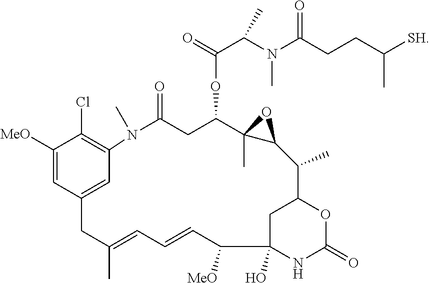 [SMILES: COc1cc2cc(c1Cl)N(C)C(=O)C[C@H](OC(=O)[C@H](C)N(C)C(=O)CCC(C)S)[C@]1(C)O[C@H]1[C@H](C)C1C[C@@](O)(NC(=O)O1)[C@H](OC)/C=C/C=C(\C)C2]

Each of the maytansinoids taught in U.S. Pat. Nos. 5,208,020 and 7,276,497, can also be used in the conjugate of the present invention. In this regard, the entire disclosure of U.S. Pat. Nos. 5,208,020 and 7,276,697 is incorporated herein by reference.

Many positions on maytansinoids can serve as the position to chemically link the linking moiety. For example, the C-3 position having a hydroxyl group, the C-14 position modified with hydroxymethyl, the C-15 position modified with hydroxy and the C-20 position having a hydroxy group are all expected to be useful. In some embodiments, the C-3 position serves as the position to chemically link the linking moiety, and in some particular embodiments, the C-3 position of maytansinol serves as the position to chemically link the linking moiety.

The invention also includes various isomers and mixtures of maytansinoids and conjugates. Certain compounds and conjugates of the present invention may exist in various stereoisomeric, enantiomeric, and diastereomeric forms. Several descriptions for producing such antibody-maytansinoid conjugates are provided in U.S. Pat. Nos. 5,208,020, 5,416,064 6,333,410, 6,441,163, 6,716,821, and 7,368,565, each of which is incorporated herein in its entirety.

A therapeutically effective number of maytansinoid molecules bound per antibody molecule can be determined by measuring spectrophotometrically the ratio of the absorbance at 252 nm and 280 nm. An average of 3 to 4 maytansinoid molecules conjugated per antibody molecule can enhancing cytotoxicity of target cells without negatively affecting the function or solubility of the antibody, although one molecule of toxin/antibody can enhance cytotoxicity over antibody alone. The average number of maytansinoid molecules/antibody or antigen binding fragment thereof, or soluble ligand, can be, for example, 1-10 or 2-5.

### Amatoxins

In some embodiments, the cytotoxin of the antibody-drug conjugate is an RNA polymerase inhibitor. In some embodiments, the RNA polymerase inhibitor is an amatoxin or derivative thereof. In some embodiments, the cytotoxin is an amatoxin or derivative thereof, such as α-amanitin, β-amanitin, γ-amanitin, ε-amanitin, amanin, amaninamide, amanullin, amanullinic acid, or proamanullin. Structures of the various naturally occurring amatoxins are represented by formulae (III), (IIIA), and (IIIB), and are disclosed in, e.g., Zanotti et al., Int. J. Peptide Protein Res. 30, 1987, 450-459.

In certain embodiments, amatoxins useful in conjunction with the compositions and methods described herein include compounds according to formulae (III), (IIIA), and (IIIB), (e.g., α-amanitin, β-amanitin, γ-amanitin, ε-amanitin, amanin, amaninamide, amanullin, amanullinic acid, proamanullin, or derivatives thereof). Formula (III) is as follows:

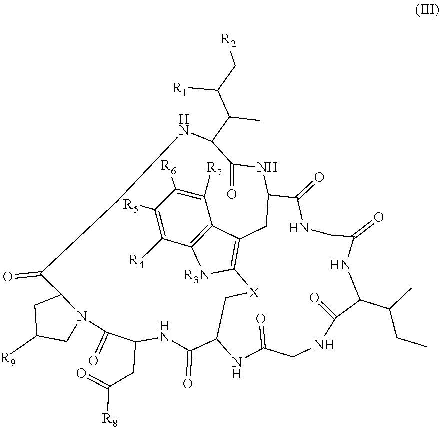

wherein R₁ is H, OH, or OR_{A};

R₂ is H, OH, or OR_{B};

R_{A} and R_{B}, when present, together with the oxygen atoms to which they are bound, combine to form an optionally substituted 5-membered heterocycloalkyl group;

R₃ is H or R_{D};

R₄ is H, OH, OR_{D}, or R_{D};

R₅ is H, OH, OR_{D}, or R_{D};

R₆ is H, OH, OR_{D}, or R_{D};

R₇ is H, OH, OR_{D}, or R_{D};

R₈ is OH, NH₂, or OR_{D};

R₉ is H, OH, or OR_{D};

X is —S—, —S(O)—, or —SO₂—; and

R_{D} is optionally substituted alkyl (e.g., C₁-C₆ alkyl), optionally substituted heteroalkyl (e.g., C₁-C₆ heteroalkyl), optionally substituted alkenyl (e.g., C₂-C₆ alkenyl), optionally substituted heteroalkenyl (e.g., C₂-C₆ heteroalkenyl), optionally substituted alkynyl (e.g., C₂-C₆ alkynyl), optionally substituted heteroalkynyl (e.g., C₂-C₆ heteroalkynyl), optionally substituted cycloalkyl, optionally substituted heterocycloalkyl, optionally substituted aryl, or optionally substituted heteroaryl.

For instance, in one embodiment, amatoxins useful in conjunction with the compositions and methods described herein include compounds according to formula (IIIA), below:

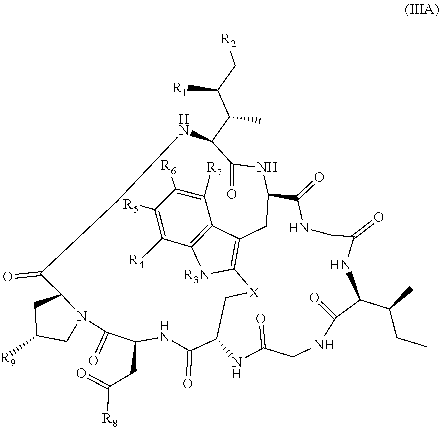

wherein R₁ is H, OH, or OR_{A};

R₂ is H, OH, or OR_{B};

R_{A} and R_{B}, when present, together with the oxygen atoms to which they are bound, combine to form an optionally substituted 5-membered heterocycloalkyl group;

R₃ is H or R_{D};

R₄ is H, OH, OR_{D}, or R_{D};

R₅ is H, OH, OR_{D}, or R_{D};

R₆ is H, OH, OR_{D}, or R_{D};

R₇ is H, OH, OR_{D}, or R_{D};

R₈ is OH, NH₂, or OR_{D};

R₉ is H, OH, or OR_{D};

X is —S—, —S(O)—, or —SO₂—; and

R_{D} is optionally substituted alkyl (e.g., C₁-C₆ alkyl), optionally substituted heteroalkyl (e.g., C₁-C₆ heteroalkyl), optionally substituted alkenyl (e.g., C₂-C₆ alkenyl), optionally substituted heteroalkenyl (e.g., C₂-C₆ heteroalkenyl), optionally substituted alkynyl (e.g., C₂-C₆ alkynyl), optionally substituted heteroalkynyl (e.g., C₂-C₆ heteroalkynyl), optionally substituted cycloalkyl, optionally substituted heterocycloalkyl, optionally substituted aryl, or optionally substituted heteroaryl.

In one embodiment, amatoxins useful in conjunction with the compositions and methods described herein also include compounds according to formula (IIIB), below:

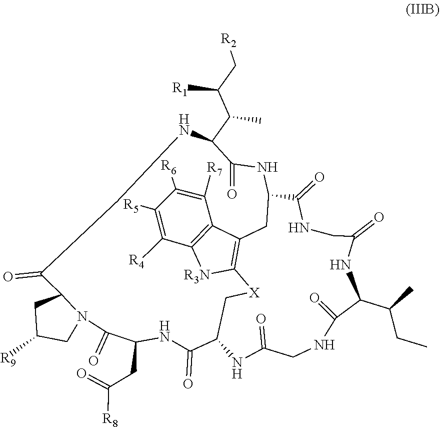

wherein R₁ is H, OH, or OR_{A};

R₂ is H, OH, or OR_{B};

R_{A} and R_{B}, when present, together with the oxygen atoms to which they are bound, combine to form an optionally substituted 5-membered heterocycloalkyl group;

R₃ is H or R_{D};

R₄ is H, OH, OR_{D}, or R_{D};

R₅ is H, OH, OR_{D}, or R_{D};

R₆ is H, OH, OR_{D}, or R_{D};

R₇ is H, OH, OR_{D}, or R_{D};

R₈ is OH, NH₂, or OR_{D};

R₉ is H, OH, or OR_{D};

X is —S—, —S(O)—, or —SO₂—; and

R_{D} is optionally substituted alkyl (e.g., C₁-C₆ alkyl), optionally substituted heteroalkyl (e.g., C₁-C₆ heteroalkyl), optionally substituted alkenyl (e.g., C₂-C₆ alkenyl), optionally substituted heteroalkenyl (e.g., C₂-C₆ heteroalkenyl), optionally substituted alkynyl (e.g., C₂-C₆ alkynyl), optionally substituted heteroalkynyl (e.g., C₂-C₆ heteroalkynyl), optionally substituted cycloalkyl, optionally substituted heterocycloalkyl, optionally substituted aryl, or optionally substituted heteroaryl.

In one embodiment, the cytotoxin is an amanitin.

As described herein, amatoxins may be conjugated to an antibody, or an antigen-binding fragment thereof, for instance, by way of a linker moiety. Exemplary methods of amatoxin conjugation and linkers useful for such processes are described in the section entitled “Linkers for chemical conjugation,” as well as in Table 1, below. Exemplary linker-containing amatoxins useful for conjugation to an anti-CD134 or anti-CD278 antibody, or an antigen-binding fragment, in accordance with the compositions and methods described herein are shown in structural formulas (I), (IA), (IB), (II), (IIA), and (IIB), recited herein.

For instance, the antibodies, or antigen-binding fragments, described herein may be bound to an amatoxin so as to form a conjugate represented by the formula Ab-Z-L-Am, wherein Ab is the antibody, or antigen-binding fragment thereof, L is a linker, Z is a chemical moiety and Am is an amatoxin. Many positions on amatoxins or derivatives thereof can serve as the position to covalently bond the linking moiety L, and, hence the antibodies or antigen-binding fragments thereof. In some embodiments, the amatoxin-linker conjugate Am-L-Z is represented by formula (I)

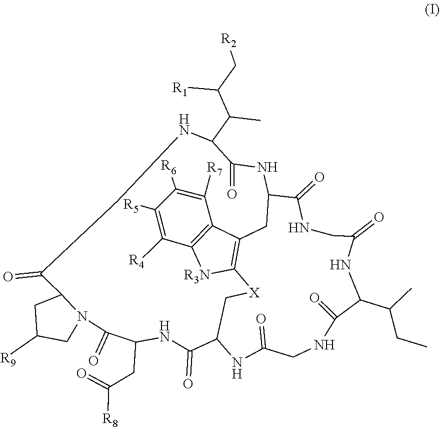

wherein R₁ is H, OH, OR_{A}, or OR_{C};

R₂ is H, OH, OR_{B}, or OR_{C};

R_{A} and R_{B}, together with the oxygen atoms to which they are bound, combine to form an optionally substituted 5-membered heterocycloalkyl group;

R₃ is H, R_{C}, or R_{D};

R₄ is H, OH, OR_{C}, OR_{D}, R_{C}, or R_{D};

R₅ is H, OH, OR_{C}, OR_{D}, R_{C}, or R_{D};

R₆ is H, OH, OR_{C}, OR_{D}, R_{C}, or R_{D};

R₇ is H, OH, OR_{C}, OR_{D}, R_{C}, or R_{D};

R₈ is OH, NH₂, OR_{C}, OR_{D}, NHR_{C}, or NR_{C}R_{D};

R₉ is H, OH, OR_{C}, or OR_{D};

X is —S—, —S(O)—, or —SO₂—;

R_{C} is L-Z;

R_{D} is optionally substituted C₁-C₆ alkyl, optionally substituted C₁-C₆ heteroalkyl, optionally substituted C₂-C₆ alkenyl, optionally substituted C₂-C₆ heteroalkenyl, optionally substituted C₂-C₆ alkynyl, optionally substituted C₂-C₆ heteroalkynyl, optionally substituted cycloalkyl, optionally substituted heterocycloalkyl, optionally substituted aryl, or optionally substituted heteroaryl;

L is a linker, such as optionally substituted C₁-C₆ alkylene, optionally substituted C₁-C₆ heteroalkylene, optionally substituted C₂-C₆ alkenylene, optionally substituted C₂-C₆ heteroalkenylene, optionally substituted C₂-C₆ alkynylene, optionally substituted C₂-C₆ heteroalkynylene, optionally substituted cycloalkylene, optionally substituted heterocycloalkylene, optionally substituted arylene, optionally substituted heteroarylene, a dipeptide, —(C═O)—, a peptide, or a combination thereof; and

Z is a chemical moiety that forms a coupling reaction between a reactive substituent present on L and a reactive substituent present within an antibody, antigen-binding fragment thereof, or soluble ligand that binds CD134 or CD278.

In some embodiments, the cytotoxin contains one R_{C} substituent.

In some embodiments, the linker comprises a —(CH)₂ₙ— unit, where n is an integer from 2-6. In some embodiments, the linker includes —((CH₂)ₙ where n is 6. In some embodiments, L-Z is

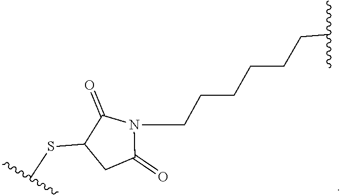

where S is a sulfur atom which represents the reactive substituent present within an antibody, or antigen-binding fragment thereof, that binds CD134 or CD278 (e.g., from the —SH group of a cysteine residue).

In some embodiments, L-Z is

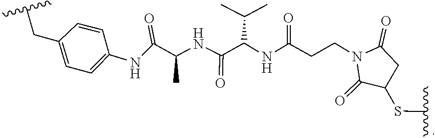

In some embodiments, Am-L-Z-Ab is

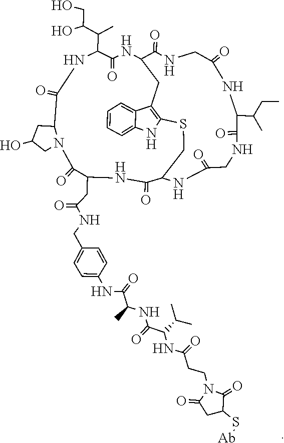

In some embodiments, Am-L-Z-Ab is

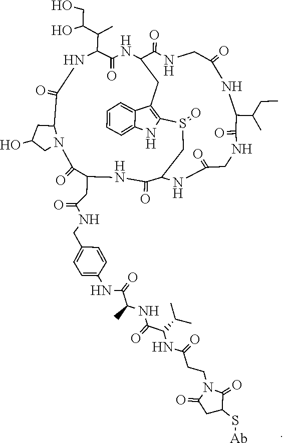

In some embodiments, Am-L-Z is represented by formula (IA)

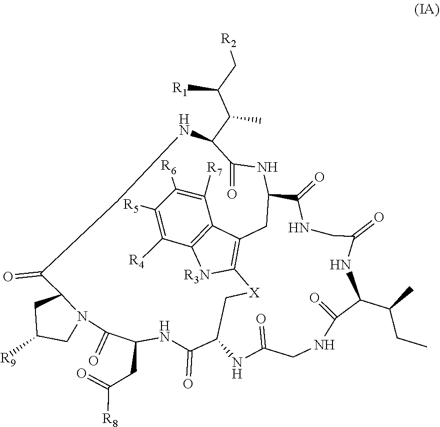

wherein R₁ is H, OH, OR_{A}, or OR_{C};

R₂ is H, OH, OR_{B}, or OR_{C};

R_{A} and R_{B}, when present, together with the oxygen atoms to which they are bound, combine to form an optionally substituted 5-membered heterocycloalkyl group;

R₃ is H, R_{C}, or R_{D};

R₄ is H, OH, OR_{C}, OR_{D}, R_{C}, or R_{D};

R₅ is H, OH, OR_{C}, OR_{D}, R_{C}, or R_{D};

R₆ is H, OH, OR_{C}, OR_{D}, R_{C}, or R_{D};

R₇ is H, OH, OR_{C}, OR_{D}, R_{C}, or R_{D};

R₈ is OH, NH₂, OR_{C}, OR_{D}, NHR_{C}, or NR_{C}R_{D};

R₉ is H, OH, OR_{C}, or OR_{D};

X is —S—, —S(O)—, or —SO₂—;

R_{C} is L-Z;

R_{D} is optionally substituted C₁-C₆ alkyl, optionally substituted C₁-C₆ heteroalkyl, optionally substituted C₂-C₆ alkenyl, optionally substituted C₂-C₆ heteroalkenyl, optionally substituted C₂-C₆ alkynyl, optionally substituted C₂-C₆ heteroalkynyl, optionally substituted cycloalkyl, optionally substituted heterocycloalkyl, optionally substituted aryl, or optionally substituted heteroaryl;

L is optionally substituted C₁-C₆ alkylene, optionally substituted C₁-C₆ heteroalkylene, optionally substituted C₂-C₆ alkenylene, optionally substituted C₂-C₆ heteroalkenylene, optionally substituted C₂-C₆ alkynylene, optionally substituted C₂-C₆ heteroalkynylene, optionally substituted cycloalkylene, optionally substituted heterocycloalkylene, optionally substituted arylene, optionally substituted heteroarylene; a dipeptide, —(C═O)—, a peptide, or a combination thereof;

Z is a chemical moiety that forms a coupling reaction between a reactive substituent present on L and a reactive substituent present within an antibody, antigen-binding fragment thereof, or soluble ligands that binds CD134 or CD278; and

wherein Am contains one R_{C} substituent.

In some embodiments, the linker includes —((CH₂)ₙ where n is 6. In some embodiments, L-Z is

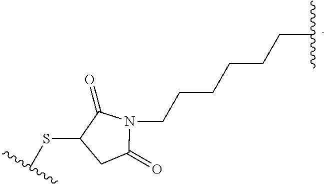 [SMILES: *CCCCCCN1C(=O)CC(S*)C1=O]

In some embodiments, L-Z is

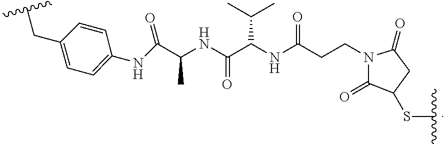

In some embodiments, Am-L-Z-Ab is

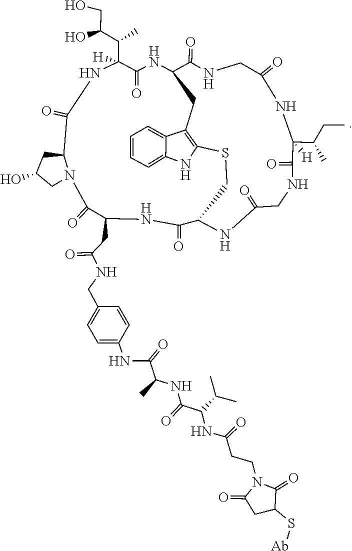

In some embodiments, Am-L-Z-Ab is

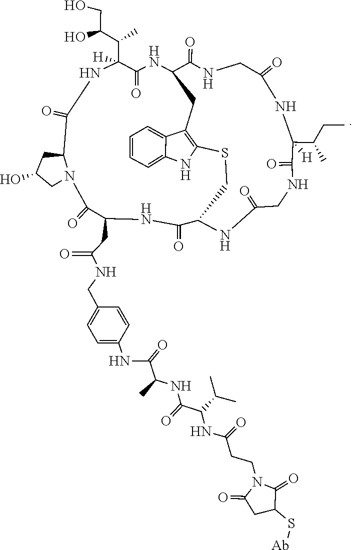

In some embodiments, Am-L-Z is represented by formula (IB)

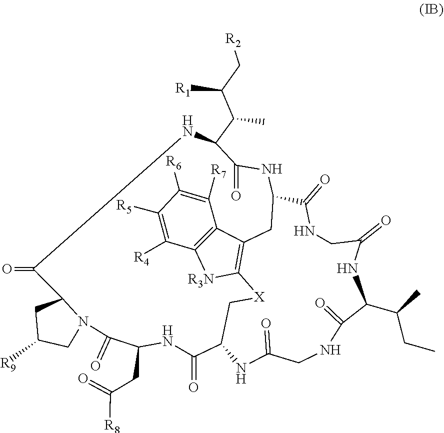

wherein R₁ is H, OH, OR_{A}, or OR_{C};

R₂ is H, OH, OR_{B}, or OR_{C};

R_{A} and R_{B}, when present, together with the oxygen atoms to which they are bound, combine to form an optionally substituted 5-membered heterocycloalkyl group;

R₃ is H, R_{C}, or R_{D};

R₄ is H, OH, OR_{C}, OR_{D}, R_{C}, or R_{D};

R₅ is H, OH, OR_{C}, OR_{D}, R_{C}, or R_{D};

R₆ is H, OH, OR_{C}, OR_{D}, R_{C}, or R_{D};

R₇ is H, OH, OR_{C}, OR_{D}, R_{C}, or R_{D};

R₈ is OH, NH₂, OR_{C}, OR_{D}, NHR_{C}, or NR_{C}R_{D};

R₉ is H, OH, OR_{C}, or OR_{D};

X is —S—, —S(O)—, or —SO₂—;

R_{C} is -L-Z;

R_{D} is optionally substituted alkyl (e.g., C₁-C₆ alkyl), optionally substituted heteroalkyl (e.g., C₁-C₆ heteroalkyl), optionally substituted alkenyl (e.g., C₂-C₆ alkenyl), optionally substituted heteroalkenyl (e.g., C₂-C₆ heteroalkenyl), optionally substituted alkynyl (e.g., C₂-C₆ alkynyl), optionally substituted heteroalkynyl (e.g., C₂-C₆ heteroalkynyl), optionally substituted cycloalkyl, optionally substituted heterocycloalkyl, optionally substituted aryl, or optionally substituted heteroaryl;

L is a linker, such as optionally substituted alkylene (e.g., C₁-C₆ alkylene), optionally substituted heteroalkylene (C₁-C₆ heteroalkylene), optionally substituted alkenylene (e.g., C₂-C₆ alkenylene), optionally substituted heteroalkenylene (e.g., C₂-C₆ heteroalkenylene), optionally substituted alkynylene (e.g., C₂-C₆ alkynylene), optionally substituted heteroalkynylene (e.g., C₂-C₆ heteroalkynylene), optionally substituted cycloalkylene, optionally substituted heterocycloalkylene, optionally substituted arylene, optionally substituted heteroarylene; a dipeptide, —(C═O)—, or a peptide, or a combination thereof;

Z is a chemical moiety formed from a coupling reaction between a reactive substituent present on L and a reactive substituent present within an antibody, or an antigen-binding fragment thereof, that binds CD134 or CD278; and

wherein Am contains exactly one R_{C} substituent.

In some embodiments, R_{A} and R_{B}, together with the oxygen atoms to which they are bound, combine to form:

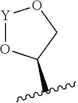

wherein Y is —(C═O)—, —(C═S)—, —(C═NR_{E)}—, or —(CR_{E}R_{E′})—; and

R_{E} and R_{E′} are each independently optionally substituted C₁-C₆ alkylene-R_{C}, optionally substituted C₁-C₆ heteroalkylene-R_{C}, optionally substituted C₂-C₆ alkenylene-R_{C}, optionally substituted C₂-C₆ heteroalkenylene-R_{C}, optionally substituted C₂-C₆ alkynylene-R_{C}, optionally substituted C₂-C₆ heteroalkynylene-R_{C}, optionally substituted cycloalkylene-R_{C}, optionally substituted heterocycloalkylene-R_{C}, optionally substituted arylene-R_{C}, or optionally substituted heteroarylene-R_{C}.

In some embodiments, Am-L-Z is represented by formula (I), (IA), or (IB)), wherein R₁ is H, OH, OR_{A}, or OR_{C};

R₂ is H, OH, OR_{B}, or OR_{C};

R_{A} and R_{B}, together with the oxygen atoms to which they are bound, combine to form:

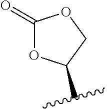 [SMILES: *[C@@H]1COC(=O)O1]

R₃ is H or R_{C};

R₄ is H, OH, OR_{C}, OR_{D}, R_{C}, or R_{D};

R₅ is H, OH, OR_{C}, OR_{D}, R_{C}, or R_{D};

R₆ is H, OH, OR_{C}, OR_{D}, R_{C}, or R_{D};

R₇ is H, OH, OR_{C}, OR_{D}, R_{C}, or R_{D};

R₈ is OH, NH₂, OR_{C}, or NHR_{C};

R₉ is H or OH; and

wherein R_{C} and R_{D} are each as defined above.

In some embodiments, Am-L-Z is represented by formula (I), (IA), or (IB),

wherein R₁ is H, OH, OR_{A}, or OR_{C};

R₂ is H, OH, OR_{B}, or OR_{C};

R_{A} and R_{B}, together with the oxygen atoms to which they are bound, combine to form:

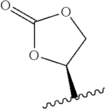 [SMILES: *[C@@H]1COC(=O)O1]

R₃ is H or R_{C};

R₄ and R₅ are each independently H, OH, OR_{C}, R_{C}, or OR_{D};

R₆ and R₇ are each H;

R₈ is OH, NH₂, OR_{C}, or NHR_{C};

R₉ is H or OH; and

wherein X and R_{C} are as defined above.

In some embodiments, Am-L-Z is represented by formula (I), (IA), or (IB)

wherein R₁ is H, OH, or OR_{A};

R₂ is H, OH, or OR_{B};

R_{A} and R_{B}, together with the oxygen atoms to which they are bound, combine to form:

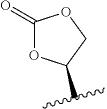 [SMILES: *[C@@H]1COC(=O)O1]

R₃, R₄, R₆, and R₇ are each H;

R₅ is OR_{C};

R₈ is OH or NH₂;

R₉ is H or OH; and

wherein R_{C} is as defined above. Such amatoxin conjugates are described, for example, in US Patent Application Publication No. 2016/0002298, the disclosure of which is incorporated herein by reference in its entirety.

In some embodiments, Am-L-Z is represented by formula (I), (IA), or (IB),

wherein R₁ and R₂ are each independently H or OH;

R₃ is R_{C};

R₄, R₆, and R₇ are each H;

R₅ is H, OH, or OC₁-C₆ alkyl;

R₈ is OH or NH₂;

R₉ is H or OH; and

wherein R_{C} is as defined above. Such amatoxin conjugates are described, for example, in US Patent Application Publication No. 2014/0294865, the disclosure of which is incorporated herein by reference in its entirety.

In some embodiments, Am-L-Z is represented by formula (I), (IA), or (IB)

wherein R₁ and R₂ are each independently H or OH;

R₃, R₆, and R₇ are each H;

R₄ and R₅ are each independently H, OH, OR_{C}, or R_{C};

R₈ is OH or NH₂;

R₉ is H or OH; and

wherein R_{C} is as defined above. Such amatoxin conjugates are described, for example, in US Patent Application Publication No. 2015/0218220, the disclosure of which is incorporated herein by reference in its entirety.

In some embodiments, Am-L-Z is represented by formula (I), (IA), or (IB)wherein R₁ and R₂ are each independently H or OH;

R₃, R₆, and R₇ are each H;

R₄ and R₅ are each independently H or OH;

R₈ is OH, NH₂, OR_{C}, or NHR_{C};

R₉ is H or OH; and

wherein R_{C} is as defined above. Such amatoxin conjugates are described, for example, in U.S. Pat. Nos. 9,233,173 and 9,399,681, the disclosures of each of which are incorporated herein by reference in their entirety. Additional amatoxins that may be used for conjugation to an antibody, or antigen-binding fragment thereof, in accordance with the compositions and methods described herein are described, for example, in WO 2016/142049; WO 2016/071856; and WO 2017/046658, the disclosures of each of which are incorporated herein by reference in their entirety.

In some embodiments, the amatoxin-linker conjugate Am-L-Z is represented by formula (II), formula (IIA), or formula (IIB)

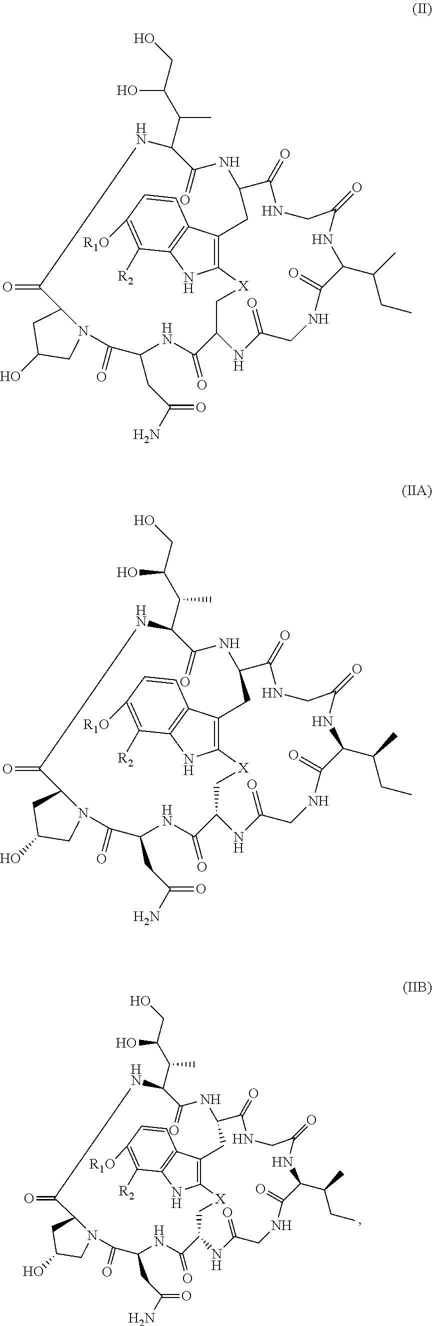

wherein X is S, SO, or SO₂; R₁ is H or a linker covalently bound to the antibody or antigen-binding fragment thereof through a chemical moiety Z, formed from a coupling reaction between a reactive substituent present on the linker and a reactive substituent present within an antibody, or antigen-binding fragment thereof; and R₂ is H or a linker covalently bound to the antibody or antigen-binding fragment thereof through a chemical moiety Z, formed from a coupling reaction between a reactive substituent present on the linker and a reactive substituent present within an antibody, or antigen-binding fragment thereof; wherein when R₁ is H, R₂ is the linker, and when R₂ is H, R₁ is the linker.

In some embodiments, the linker includes a —(CH₂)ₙ— unit, where n is an integer from 2-6. In some embodiments, R₁ is the linker and R₂ is H, and the linker and chemical moiety, together as L-Z, is

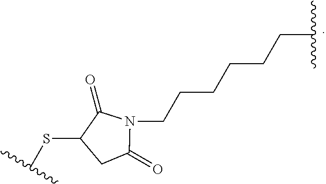 [SMILES: *CCCCCCN1C(=O)CC(S*)C1=O]

In some embodiments, Am-L-Z-Ab is one of:

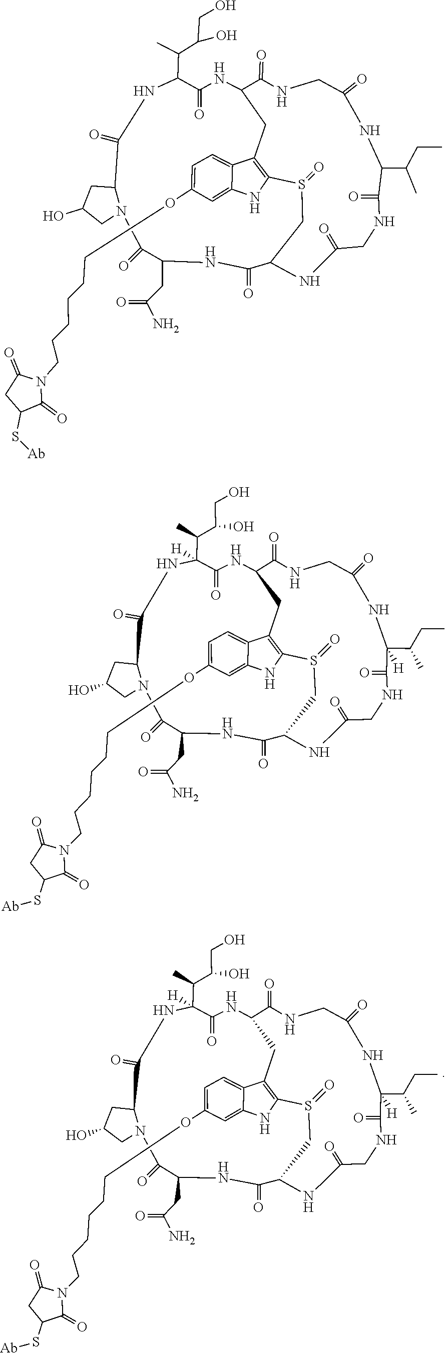

In some embodiments, the cytotoxin is an amatoxin. In some embodiments, the amatoxin is a compound of formula (III), (IIIA), or (IIIB). In some embodiments, the amatoxin of formula (III), (IIIA), or (IIIB) is attached to an anti-CD134 or CD278 antibody via a linker L. The linker L may be attached to the amatoxin of formula (III), (IIIA), or (IIIB) at any one of several possible positions (e.g., any of R¹-R⁹) to provide an amatoxin-linker conjugate of formula (I), (IA), (IB), (II), (IIA), or (IIB). In some embodiments, the linker is attached at position R¹. In some embodiments, the linker is attached at position R². In some embodiments, the linker is attached at position R³. In some embodiments, the linker is attached at position R⁴. In some embodiments, the linker is attached at position R⁵. In some embodiments, the linker is attached at position R⁶. In some embodiments, the linker is attached at position R⁷. In some embodiments, the linker is attached at position R⁸. In some embodiments, the linker is attached at position R⁹. In some embodiments, the cytotoxin is an α-amanitin. In some embodiments, the linker includes a hydrazine, a disulfide, a thioether or a dipeptide. In some embodiments, the linker includes a dipeptide selected from Val-Ala and Val-Cit. In some embodiments, the linker includes a para-aminobenzyl group (PAB). In some embodiments, the linker includes the moiety PAB-Cit-Val. In some embodiments, the linker includes the moiety PAB-Ala-Val. In some embodiments, the linker includes a —((C═O)(CH₂)ₙ— unit, wherein n is an integer from 1-6.

In some embodiments, the linker includes a —(CH₂)ₙ— unit, where n is an integer from 2-6. In some embodiments, the linker is -PAB-Cit-Val-((C═O)(CH₂)ₙ—. In some embodiments, the linker is -PAB-Ala-Val-((C═O)(CH₂)ₙ—. In some embodiments, the linker L and the chemical moiety Z, taken together as L-Z, is

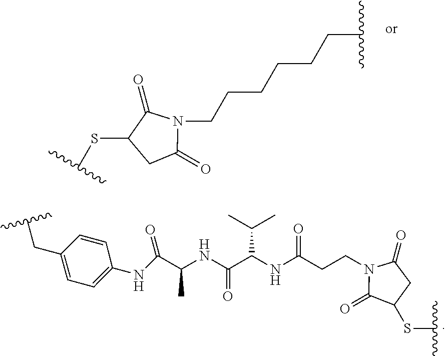

In some embodiments, the cytotoxin is a β-amanitin. In some embodiments, the β-amanitin is a compound of formula IV. In some embodiments, the β-amanitin of formula IV is attached to an anti-CD134 antibody via a linker L. The linker L may be attached to the β-amanitin of formula IV at any one of several possible positions (e.g., any of R¹-R⁹). In some embodiments, the linker is attached at position R¹. In some embodiments, the linker is attached at position R². In some embodiments, the linker is attached at position R³. In some embodiments, the linker is attached at position R⁴. In some embodiments, the linker is attached at position R⁵. In some embodiments, the linker is attached at position R⁶. In some embodiments, the linker is attached at position R⁷. In some embodiments, the linker is attached at position R⁸. In some embodiments, the linker is attached at position R⁹. In some embodiments, the linker includes a hydrazine, a disulfide, a thioether or a dipeptide. In some embodiments, the linker includes a dipeptide selected from Val-Ala and Val-Cit. In some embodiments, the linker includes a para-aminobenzyl group (PAB). In some embodiments, the linker includes the moiety PAB-Cit-Val. In some embodiments, the linker includes the moiety PAB-Ala-Val. In some embodiments, the linker includes a —((C═O)(CH₂)ₙ— unit, wherein n is an integer from 1-6. In some embodiments, the linker is -PAB-Cit-Val-((C═O)(CH₂)ₙ—. In some embodiments, the linker is -PAB-Ala-Val-((C═O)(CH₂)ₙ—.

In some embodiments, the cytotoxin is a γ-amanitin. In some embodiments, the γ-amanitin is a compound of formula IV. In some embodiments, the γ-amanitin of formula IV is attached to an anti-CD134 antibody via a linker L. The linker L may be attached to the γ-amanitin of formula IV at any one of several possible positions (e.g., any of R¹-R⁹). In some embodiments, the linker is attached at position R¹. In some embodiments, the linker is attached at position R². In some embodiments, the linker is attached at position R³. In some embodiments, the linker is attached at position R⁴. In some embodiments, the linker is attached at position R⁵. In some embodiments, the linker is attached at position R⁶. In some embodiments, the linker is attached at position R⁷. In some embodiments, the linker is attached at position R⁸. In some embodiments, the linker is attached at position R⁹. In some embodiments, the linker includes a hydrazine, a disulfide, a thioether or a dipeptide. In some embodiments, the linker includes a dipeptide selected from Val-Ala and Val-Cit. In some embodiments, the linker includes a para-aminobenzyl group (PAB). In some embodiments, the linker includes the moiety PAB-Cit-Val. In some embodiments, the linker includes the moiety PAB-Ala-Val. In some embodiments, the linker includes a —((C═O)(CH₂)ₙ— unit, wherein n is an integer from 1-6. In some embodiments, the linker is -PAB-Cit-Val-((C═O)(CH₂)ₙ—. In some embodiments, the linker is -PAB-Ala-Val-((C═O)(CH₂)ₙ—.

In some embodiments, the cytotoxin is a ε-amanitin. In some embodiments, the ε-amanitin is a compound of formula IV. In some embodiments, the ε-amanitin of formula IV is attached to an anti-CD134 antibody via a linker L. The linker L may be attached to the ε-amanitin of formula IV at any one of several possible positions (e.g., any of R¹-R⁹). In some embodiments, the linker is attached at position R¹. In some embodiments, the linker is attached at position R². In some embodiments, the linker is attached at position R³. In some embodiments, the linker is attached at position R⁴. In some embodiments, the linker is attached at position R⁵. In some embodiments, the linker is attached at position R⁶. In some embodiments, the linker is attached at position R⁷. In some embodiments, the linker is attached at position R⁸. In some embodiments, the linker is attached at position R⁹. In some embodiments, the linker includes a hydrazine, a disulfide, a thioether or a dipeptide. In some embodiments, the linker includes a dipeptide selected from Val-Ala and Val-Cit. In some embodiments, the linker includes a para-aminobenzyl group (PAB). In some embodiments, the linker includes the moiety PAB-Cit-Val. In some embodiments, the linker includes the moiety PAB-Ala-Val. In some embodiments, the linker includes a —((C═O)(CH₂)ₙ— unit, wherein n is an integer from 1-6. In some embodiments, the linker is -PAB-Cit-Val-((C═O)(CH₂)ₙ—. In some embodiments, the linker is -PAB-Ala-Val-((C═O)(CH₂)ₙ—.

In some embodiments, the cytotoxin is an amanin. In some embodiments, the amanin is a compound of formula IV. In some embodiments, the amanin of formula IV is attached to an anti-CD134 antibody via a linker L. The linker L may be attached to the amanin of formula IV at any one of several possible positions (e.g., any of R¹-R⁹). In some embodiments, the linker is attached at position R¹. In some embodiments, the linker is attached at position R². In some embodiments, the linker is attached at position R³. In some embodiments, the linker is attached at position R⁴. In some embodiments, the linker is attached at position R⁵. In some embodiments, the linker is attached at position R⁶. In some embodiments, the linker is attached at position R⁷. In some embodiments, the linker is attached at position R⁸. In some embodiments, the linker is attached at position R⁹. In some embodiments, the linker includes a hydrazine, a disulfide, a thioether or a dipeptide. In some embodiments, the linker includes a dipeptide selected from Val-Ala and Val-Cit. In some embodiments, the linker includes a para-aminobenzyl group (PAB). In some embodiments, the linker includes the moiety PAB-Cit-Val. In some embodiments, the linker includes the moiety PAB-Ala-Val. In some embodiments, the linker includes a —((C═O)(CH₂)ₙ— unit, wherein n is an integer from 1-6. In some embodiments, the linker is -PAB-Cit-Val-((C═O)(CH₂)ₙ—. In some embodiments, the linker is -PAB-Ala-Val-((C═O)(CH₂)ₙ—.

In some embodiments, the cytotoxin is an amaninamide. In some embodiments, the amaninamide is a compound of formula IV. In some embodiments, the amaninamide of formula IV is attached to an anti-CD134 antibody via a linker L. The linker L may be attached to the amaninamide of formula IV at any one of several possible positions (e.g., any of R¹-R⁹). In some embodiments, the linker is attached at position R¹. In some embodiments, the linker is attached at position R². In some embodiments, the linker is attached at position R³. In some embodiments, the linker is attached at position R⁴. In some embodiments, the linker is attached at position R⁵. In some embodiments, the linker is attached at position R⁶. In some embodiments, the linker is attached at position R⁷. In some embodiments, the linker is attached at position R⁸. In some embodiments, the linker is attached at position R⁹. In some embodiments, the linker includes a hydrazine, a disulfide, a thioether or a dipeptide. In some embodiments, the linker includes a dipeptide selected from Val-Ala and Val-Cit. In some embodiments, the linker includes a para-aminobenzyl group (PAB). In some embodiments, the linker includes the moiety PAB-Cit-Val. In some embodiments, the linker includes the moiety PAB-Ala-Val. In some embodiments, the linker includes a —((C═O)(CH₂)ₙ— unit, wherein n is an integer from 1-6. In some embodiments, the linker is -PAB-Cit-Val-((C═O)(CH₂)ₙ—. In some embodiments, the linker is -PAB-Ala-Val-((C═O)(CH₂)ₙ—.

In some embodiments, the cytotoxin is an amanullin. In some embodiments, the amanullin is a compound of formula IV. In some embodiments, the amanullin of formula IV is attached to an anti-CD134 antibody via a linker L. The linker L may be attached to the amanullin of formula IV at any one of several possible positions (e.g., any of R¹-R⁹). In some embodiments, the linker is attached at position R¹. In some embodiments, the linker is attached at position R². In some embodiments, the linker is attached at position R³. In some embodiments, the linker is attached at position R⁴. In some embodiments, the linker is attached at position R⁵. In some embodiments, the linker is attached at position R⁶. In some embodiments, the linker is attached at position R⁷. In some embodiments, the linker is attached at position R⁸. In some embodiments, the linker is attached at position R⁹. In some embodiments, the linker includes a hydrazine, a disulfide, a thioether or a dipeptide. In some embodiments, the linker includes a dipeptide selected from Val-Ala and Val-Cit. In some embodiments, the linker includes a para-aminobenzyl group (PAB). In some embodiments, the linker includes the moiety PAB-Cit-Val. In some embodiments, the linker includes the moiety PAB-Ala-Val. In some embodiments, the linker includes a —((C═O)(CH₂)ₙ— unit, wherein n is an integer from 1-6. In some embodiments, the linker is -PAB-Cit-Val-((C═O)(CH₂)ₙ—. In some embodiments, the linker is -PAB-Ala-Val-((C═O)(CH₂)ₙ—.

In some embodiments, the cytotoxin is an amanullinic acid. In some embodiments, the amanullinic acid is a compound of formula IV. In some embodiments, the amanullinic acid of formula IV is attached to an anti-CD134 antibody via a linker L. The linker L may be attached to the amanullinic acid of formula IV at any one of several possible positions (e.g., any of R¹-R⁹). In some embodiments, the linker is attached at position R¹. In some embodiments, the linker is attached at position R². In some embodiments, the linker is attached at position R³. In some embodiments, the linker is attached at position R⁴. In some embodiments, the linker is attached at position R⁵. In some embodiments, the linker is attached at position R⁶. In some embodiments, the linker is attached at position R⁷. In some embodiments, the linker is attached at position R⁸. In some embodiments, the linker is attached at position R⁹. In some embodiments, the linker includes a hydrazine, a disulfide, a thioether or a dipeptide. In some embodiments, the linker includes a dipeptide selected from Val-Ala and Val-Cit. In some embodiments, the linker includes a para-aminobenzyl group (PAB). In some embodiments, the linker includes the moiety PAB-Cit-Val. In some embodiments, the linker includes the moiety PAB-Ala-Val. In some embodiments, the linker includes a —((C═O)(CH₂)ₙ— unit, wherein n is an integer from 1-6. In some embodiments, the linker is -PAB-Cit-Val-((C═O)(CH₂)ₙ—. In some embodiments, the linker is -PAB-Ala-Val-((C═O)(CH₂)ₙ—.

In some embodiments, the cytotoxin is a proamanullin. In some embodiments, the proamanullin is a compound of formula IV. In some embodiments, the proamanullin of formula IV is attached to an anti-CD134 antibody via a linker L. The linker L may be attached to the proamanullin of formula IV at any one of several possible positions (e.g., any of R¹-R⁹). In some embodiments, the linker is attached at position R¹. In some embodiments, the linker is attached at position R². In some embodiments, the linker is attached at position R³. In some embodiments, the linker is attached at position R⁴. In some embodiments, the linker is attached at position R⁵. In some embodiments, the linker is attached at position R⁶. In some embodiments, the linker is attached at position R⁷. In some embodiments, the linker is attached at position R⁸. In some embodiments, the linker is attached at position R⁹. In some embodiments, the linker includes a hydrazine, a disulfide, a thioether or a dipeptide. In some embodiments, the linker includes a dipeptide selected from Val-Ala and Val-Cit. In some embodiments, the linker includes a para-aminobenzyl group (PAB). In some embodiments, the linker includes the moiety PAB-Cit-Val. In some embodiments, the linker includes the moiety PAB-Ala-Val. In some embodiments, the linker includes a —((C═O)(CH₂)ₙ— unit, wherein n is an integer from 1-6. In some embodiments, the linker is -PAB-Cit-Val-((C═O)(CH₂)ₙ—. In some embodiments, the linker is -PAB-Ala-Val-((C═O)(CH₂)ₙ—.

Antibodies, antigen-binding fragments, and ligands for use with the compositions and methods described herein can be conjugated to an amatoxin, such as α-amanitin or a variant thereof using conjugation techniques known in the art or described herein. For instance, antibodies, antigen-binding fragments thereof, and ligands that recognize and bind CD134 or CD278 can be conjugated to α-amanitin or a variant thereof, as described in US 2015/0218220, the disclosure of which is incorporated herein by reference as it pertains, for example, to amatoxins, such as α-amanitin and variants thereof, as well as covalent linkers that can be used for covalent conjugation. Synthetic methods of making amatoxins are described in, for example, U.S. Pat. No. 9,676,702, which is incorporated by reference herein with respect to the synthetic methods disclosed therein.

Exemplary antibody-drug conjugates and ligand-drug conjugates useful in conjunction with the methods described herein may be formed by the reaction of an antibody, antigen-binding fragment thereof, or ligand with an amatoxin that is conjugated to a linker containing a substituent suitable for reaction with a reactive residue on the antibody, antigen-binding fragment thereof, or ligand. Amatoxins that are conjugated to a linker containing a substituent suitable for reaction with a reactive residue on the antibody, antigen-binding fragment thereof, or ligand include, without limitation, 7′C-(4-(6-(maleimido)hexanoyl)piperazin-1-yl)-amatoxin; 7′C-(4-(6-(maleimido)hexanamido)piperidin-1-yl)-amatoxin; 7′C-(4-(6-(6-(maleimido)hexanamido)hexanoyl)piperazin-1-yl)-amatoxin; 7′C-(4-(4-((maleimido)methyl)cyclohexanecarbonyl)piperazin-1-yl)-amatoxin; 7′C-(4-(6-(4-((maleimido)methyl)cyclohexanecarboxamido)hexanoyl)piperazin-1-yl)-amatoxin; 7′C-(4-(2-(6-(maleimido)hexanamido)ethyl)piperidin-1-yl)-amatoxin; 7′C-(4-(2-(6-(6-(maleimido)hexanamido)hexanamido)ethyl)piperidin-1-yl)-amatoxin; 7′C-(4-(2-(4-((maleimido)methyl)cyclohexanecarboxamido)ethyl)piperidin-1-yl)-amatoxin; 7′C-(4-(2-(6-(4-((maleimido)methyl)cyclohexanecarboxamido)hexanamido)ethyl)piperidin-1-yl)-amatoxin; 7′C-(4-(2-(3-carboxypropanamido)ethyl)piperidin-1-yl)-amatoxin; 7′C-(4-(2-(2-bromoacetamido)ethyl)piperidin-1-yl)-amatoxin; 7′C-(4-(2-(3-(pyridin-2-yldisulfanyl)propanamido)ethyl)piperidin-1-yl)-amatoxin; 7′C-(4-(2-(4-(maleimido)butanamido)ethyl)piperidin-1-yl)-amatoxin; 7′C-(4-(2-(maleimido)acetyl)piperazin-1-yl)-amatoxin; 7′C-(4-(3-(maleimido)propanoyl)piperazin-1-yl)-amatoxin; 7′C-(4-(4-(maleimido)butanoyl)piperazin-1-yl)-amatoxin; 7′C-(4-(2-(6-(4-((maleimido)methyl)cyclohexanecarboxamido)hexanamido)ethyl)piperidin-1-yl)-amatoxin; 7′C-(3-((6-(maleimido)hexanamido)methyl)pyrrolidin-1-yl)-amatoxin; 7′C-((4-(6-(6-(maleimido)hexanamido)hexanamido)methyl)pyrrolidin-1-yl)-amatoxin; 7′C-(3-((4-((maleimido)methyl)cyclohexanecarboxamido)methyl)pyrrolidin-1-yl)-amatoxin; 7′C-(3-((6-((4-(maleimido)methyl)cyclohexanecarboxamido)hexanamido)methyl)pyrrolidin-1-yl)-amatoxin; 7′C-(4-(2-(6-(2-(aminooxy)acetamido)hexanamido)ethyl)piperidin-1-yl)-amatoxin; 7′C-(4-(2-(4-(2-(aminooxy)acetamido)butanamido)ethyl)piperidin-1-yl)-amatoxin; 7′C-(4-(4-(2-(aminooxy)acetamido)butanoyl)piperazin-1-yl)-amatoxin; 7′C-(4-(6-(2-(aminooxy)acetamido)hexanoyl)piperazin-1-yl)-amatoxin; 7′C-((4-(6-(maleimido)hexanamido)piperidin-1-yl)methyl)-amatoxin; 7′C-((4-(2-(6-(maleimido)hexanamido)ethyl)piperidin-1-yl)methyl)-amatoxin; 7′C-((4-(6-(maleimiclo)hexanoyl)piperazin-1-yl)methyl)-amatoxin; (R)-7′C-((3-((6-(maleimiclo)hexanamiclo)methyl)pyrrolidin-1-yl)methyl)-amatoxin; (S)-7′C-((3-((6-(maleimiclo)hexanamiclo)methyl)pyrrolidin-1-yl)methyl)-amatoxin; 7′C-((4-(2-(6-(6-(maleimido)hexanamido)hexanamido)ethyl)piperidin-1-yl)methyl)-amatoxin; 7′C-((4-(2-(4-((maleimido)methyl)cyclohexanecarboxamido)ethyl)piperidin-1-yl)methyl)-amatoxin; 7′C-((4-(2-(6-(4-((maleimido)methyl)cyclohexanecarboxamido)hexanamido)ethyl)piperidin-1-yl)methyl)-amatoxin; 7′C-((4-(2-(6-(maleimido)hexanamido)ethyl)piperazin-1-yl)methyl)-amatoxin; 7′C-((4-(2-(6-(6-(maleimido)hexanamido)hexanamido)ethyl)piperazin-1-yl)methyl)-amatoxin; 7′C-((4-(2-(4-((maleimido)methyl)cyclohexanecarboxamido)ethyl)piperazin-1-yl)methyl)-amatoxin; 7′C-((4-(2-(6-(4-((maleimido)methyl)cyclohexanecarboxamido)hexanamido)ethyl)piperazin-1-yl)methyl)-amatoxin; 7′C-((3-((6-(6-(maleimiclo)hexanamiclo)hexanamido)-S-methyl)pyrrolidin-1-yl)methyl)-amatoxin; 7′C-((3-((6-(6-(maleimido)hexanamido)hexanamido)-R-methyl)pyrrolidin-1-yl)methyl)-amatoxin; 7′C-((3-((4-((maleimido)methyl)cyclohexanecarboxamido)-S-methyl)pyrrolidin-1-yl)methyl)-amatoxin; 7′C-((3-((4-((maleimido)methyl)cyclohexanecarboxamido)-R-methyl)pyrrolidin-1-yl)methyl)-amatoxin; 7′C-((3-((6-(4-((maleimido)methyl)cyclohexanecarboxamido)hexanamido)methyl)pyrrolidin-1-yl)methyl)-amatoxin; 7′C-((4-(2-(3-carboxpropanamido)ethyl)piperazin-1-yl)methyl)-amatoxin; 7′C-((4-(6-(6-(maleimido)hexanamido)hexanoyl)piperazin-1-yl)methyl)-amatoxin; 7′C-((4-(6-(4-((maleimido)methyl)cyclohexanecarboxamido)hexanoyl)piperazin-1-yl)methyl)-amatoxin; 7′C-((4-(2-(maleimido)acetyl)piperazin-1-yl)methyl)-amatoxin; 7′C-((4-(3-(maleimido)propanoyl)piperazin-1-yl)methyl)-amatoxin; 7′C-((4-(4-(maleimido)butanoyl)piperazin-1-yl)methyl)-amatoxin; 7′C-((4-(2-(2-(maleimido)acetamido)ethyl)piperidin-1-yl)methyl)-amatoxin; 7′C-((4-(2-(4-(maleimido)butanamido)ethyl)piperidin-1-yl)methyl)-amatoxin; 7′C-((4-(2-(6-(4-((maleimido)methyl)cyclohexanecarboxamido)hexanamido)ethyl)piperidin-1-yl)methyl)-amatoxin; 7′C-((3-((6-(maleimido)hexanamido)methyl)azetidin-1-yl)methyl)-amatoxin; 7′C-((3-(2-(6-(maleimido)hexanamido)ethyl)azetidin-1-yl)methyl)-amatoxin; 7′C-((3-((4-((maleimido)methyl)cyclohexanecarboxamido)methyl)azetidin-1-yl)methyl)-amatoxin; 7′C-((3-(2-(4-((maleimido)methyl)cyclohexanecarboxamido)ethyl)azetidin-1yl)methyl)-amatoxin; 7′C-((3-(2-(6-(4-((maleimido)methyl)cyclohexanecarboxamido)hexanamido)ethyl)azetidin-1-yl)methyl)-amatoxin; 7′C-(((2-(6-(maleimido)-N-methylhexanamido)ethyl)(methyl)amino)methyl)-amatoxin; 7′C-(((4-(6-(maleimido)-N-methylhexanamido)butyl(methyl)amino)methyl)-amatoxin; 7′C-((2-(2-(6-(maleimido)hexanamido)ethyl)aziridin-1-yl)methyl)-amatoxin; 7′C-((2-(2-(6-(4-((maleimido)methyl)cyclohexanecarboxamido)hexanamido)ethyl)aziridin-1-yl)methyl)-amatoxin; 7′C-((4-(6-(6-(2-(aminooxy)acetamido)hexanamido)hexanoyl)piperazin-1-yl)methyl)-amatoxin; 7′C-((4-(1-(aminooxy)-2-oxo-6,9,12,15-tetraoxa-3-azaheptadecan-17-oyl)piperazin-1-yl)methyl)-amatoxin; 7′C-((4-(2-(2-(aminooxy)acetamido)acetyl)piperazin-1-yl)methyl)-amatoxin; 7′C-((4-(3-(2-(aminooxy)acetamido)propanoyl)piperazin-1-yl)methyl)-amatoxin; 7′C-((4-(4-(2-(aminooxy)acetamido)butanoyl)piperazin-1-yl)methyl)-amatoxin; 7′C-((4-(2-(6-(2-(aminooxy)acetamido)hexanamido)ethyl)piperidin-1-yl)methyl)-amatoxin; 7′C-((4-(2-(2-(2-(aminooxy)acetamido)acetamido)ethyl)piperidin-1-yl)methyl)-amatoxin; 7′C-((4-(2-(4-(2-(aminooxy)acetamido)butanamido)ethyl)piperidin-1-yl)methyl)-amatoxin; 7′C-((4-(20-(aminooxy)-4,19-dioxo-6,9,12,15-tetraoxa-3,18-diazaicosyl)piperidin-1-yl)methyl)-amatoxin; 7′C-(((2-(6-(2-(aminooxy)acetamido)-N-methylhexanamido)ethyl)(methyl)amino)methyl)-amatoxin; 7′C-(((4-(6-(2-(aminooxy)acetamido)-N-methylhexanamido)butyl)(methyl)amino)methyl)-amatoxin; 7′C-((3-((6-(4-((maleimido)methyl)cyclohexanecarboxamido)hexanamido)methyl)pyrrolidin-1-yl)-S-methyl)-amatoxin; 7′C-((3-((6-(4-((maleimido)methyl)cyclohexanecarboxamido)hexanamido)-R-methyl)pyrrolidin-1-yl)methyl)-amatoxin; 7′C-((4-(2-(2-bromoacetamido)ethyl)piperazin-1-yl)methyl)-amatoxin; 7′C-((4-(2-(2-bromoacetamido)ethyl)piperidin-1-yl)methyl)-amatoxin; 7′C-((4-(2-(3-(pyridine-2-yldisulfanyl)propanamido)ethyl)piperidin-1-yl)methyl)-amatoxin; 6′O-(6-(6-(maleimido)hexanamido)hexyl)-amatoxin; 6′O-(5-(4-((maleimido)methyl)cyclohexanecarboxamido)pentyl)-amatoxin; 6′O-(2-((6-(maleimido)hexyl)oxy)-2-oxoethyl)-amatoxin; 6′O-((6-(maleimido)hexyl)carbamoyl)-amatoxin; 6′O-((6-(4-((maleimido)methyl)cyclohexanecarboxamido)hexl)carbamoyl)-amatoxin; 6′O-(6-(2-bromoacetamido)hexyl)-amatoxin; 7′C-(4-(6-(azido)hexanamido)piperidin-1-yl)-amatoxin; 7′C-(4-(hex-5-ynoylamino)piperidin-1-yl)-amatoxin; 7′C-(4-(2-(6-(maleimido)hexanamido)ethyl)piperazin-1-yl)-amatoxin; 7′C-(4-(2-(6-(6-(maleimido)hexanamido)hexanamido)ethyl)piperazin-1-yl)-amatoxin; 6′O(6-(6-(11,12-didehydro-5,6-dihydro-dibenz[b,f]azocin-5-yl)-6-oxohexanamido)hexyl)-amatoxin; 6′O-(6-(hex-5-ynoylamino)hexyl)-amatoxin; 6′O-(6-(2-(aminooxy)acetylamido)hexyl)-amatoxin; 6′O-((6-aminooxy)hexyl)-amatoxin; and 6′O-(6-(2-iodoacetamido)hexyl)-amatoxin. The foregoing linkers, among others useful in conjunction with the compositions and methods described herein, are described, for example, in US Patent Application Publication No. 2015/0218220, the disclosure of which is incorporated herein by reference in its entirety.

Additional cytotoxins that can be conjugated to antibodies, antigen-binding fragments thereof, and ligands that recognize and bind CD134 or CD278 for use in treatment of GVHD or an autoimmune disease include, without limitation, 5-ethynyluracil, abiraterone, acylfulvene, adecypenol, adozelesin, aldesleukin, altretamine, ambamustine, amidox, amifostine, aminolevulinic acid, amrubicin, amsacrine, anagrelide, anastrozole, andrographolide, angiogenesis inhibitors, antarelix, anti-dorsalizing morphogenetic protein-1, antiandrogen, prostatic carcinoma, antiestrogen, antineoplaston, antisense oligonucleotides, aphidicolin glycinate, apoptosis gene modulators, apoptosis regulators, apurinic acid, asulacrine, atamestane, atrimustine, axinastatin 1, axinastatin 2, axinastatin 3, azasetron, azatoxin, azatyrosine, baccatin III derivatives, balanol, batimastat, BCR/ABL antagonists, benzochlorins, benzoylstaurosporine, beta lactam derivatives, beta-alethine, betaclamycin B, betulinic acid, bFGF inhibitors, bicalutamide, bisantrene, bisaziridinylspermine, bisnafide, bistratene A, bizelesin, breflate, bleomycin A2, bleomycin B2, bropirimine, budotitane, buthionine sulfoximine, calcipotriol, calphostin C, camptothecin derivatives (e.g., 10-hydroxy-camptothecin), capecitabine, carboxamide-amino-triazole, carboxyamidotriazole, carzelesin, casein kinase inhibitors, castanospermine, cecropin B, cetrorelix, chlorins, chloroquinoxaline sulfonamide, cicaprost, cis-porphyrin, cladribine, clomifene and analogues thereof, clotrimazole, collismycin A, collismycin B, combretastatin A4, combretastatin analogues, conagenin, crambescidin 816, crisnatol, cryptophycin 8, cryptophycin A derivatives, curacin A, cyclopentanthraquinones, cycloplatam, cypemycin, cytarabine ocfosfate, cytolytic factor, cytostatin, dacliximab, decitabine, dehydrodidemnin B, 2′deoxycoformycin (DCF), deslorelin, dexifosfamide, dexrazoxane, dexverapamil, diaziquone, didemnin B, didox, diethylnorspermine, dihydro-5-azacytidine, dihydrotaxol, dioxamycin, diphenyl spiromustine, discodermolide, docosanol, dolasetron, doxifluridine, droloxifene, dronabinol, duocarmycin SA, ebselen, ecomustine, edelfosine, edrecolomab, eflornithine, elemene, emitefur, epothilones, epithilones, epristeride, estramustine and analogues thereof, etoposide, etoposide 4′-phosphate (also referred to as etopofos), exemestane, fadrozole, fazarabine, fenretinide, filgrastim, finasteride, flavopiridol, flezelastine, fluasterone, fludarabine, fluorodaunorunicin hydrochloride, forfenimex, formestane, fostriecin, fotemustine, gadolinium texaphyrin, gallium nitrate, galocitabine, ganirelix, gelatinase inhibitors, gemcitabine, glutathione inhibitors, hepsulfam, homoharringtonine (HHT), hypericin, ibandronic acid, idoxifene, idramantone, ilmofosine, ilomastat, imidazoacridones, imiquimod, immunostimulant peptides, iobenguane, iododoxorubicin, ipomeanol, irinotecan, iroplact, irsogladine, isobengazole, jasplakinolide, kahalalide F, lamellarin-N triacetate, lanreotide, leinamycin, lenograstim, lentinan sulfate, leptolstatin, letrozole, lipophilic platinum compounds, lissoclinamide 7, lobaplatin, lometrexol, lonidamine, losoxantrone, loxoribine, lurtotecan, lutetium texaphyrin, lysofylline, masoprocol, maspin, matrix metalloproteinase inhibitors, menogaril, merbarone, meterelin, methioninase, metoclopramide, MIF inhibitor, ifepristone, miltefosine, mirimostim, mithracin, mitoguazone, mitolactol, mitomycin and analogues thereof, mitonafide, mitoxantrone, mofarotene, molgramostim, mycaperoxide B, myriaporone, N-acetyldinaline, N-substituted benzamides, nafarelin, nagrestip, napavin, naphterpin, nartograstim, nedaplatin, nemorubicin, neridronic acid, nilutamide, nisamycin, nitrullyn, octreotide, okicenone, onapristone, ondansetron, oracin, ormaplatin, oxaliplatin, oxaunomycin, paclitaxel and analogues thereof, palauamine, palmitoylrhizoxin, pamidronic acid, panaxytriol, panomifene, parabactin, pazelliptine, pegaspargase, peldesine, pentosan polysulfate sodium, pentostatin, pentrozole, perflubron, perfosfamide, phenazinomycin, picibanil, pirarubicin, piritrexim, podophyllotoxin, porfiromycin, purine nucleoside phosphorylase inhibitors, raltitrexed, rhizoxin, rogletimide, rohitukine, rubiginone B1, ruboxyl, safingol, saintopin, sarcophytol A, sargramostim, sobuzoxane, sonermin, sparfosic acid, spicamycin D, spiromustine, stipiamide, sulfinosine, tallimustine, tegafur, temozolomide, teniposide, thaliblastine, thiocoraline, tirapazamine, topotecan, topsentin, triciribine, trimetrexate, veramine, vinorelbine, vinxaltine, vorozole, zeniplatin, and zilascorb, among others.

### Linkers for Chemical Conjugation

A variety of linkers can be used to conjugate antibodies, antigen-binding fragments, and ligands described herein (e.g., antibodies, antigen-binding fragments thereof, and soluble ligands that recognize and bind CD134 or CD278) with a cytotoxic molecule. The term “Linker” as used herein means a divalent chemical moiety comprising a covalent bond or a chain of atoms that covalently attaches an antibody or fragment thereof (Ab) to a drug moiety (D) to form antibody-drug conjugates of the present disclosure (ADCs; Ab-Z-L-D, where D is a cytotoxin). Suitable linkers have two reactive termini, one for conjugation to an antibody and the other for conjugation to a cytotoxin. The antibody conjugation reactive terminus of the linker (reactive moiety, Z) is typically a site that is capable of conjugation to the antibody through a cysteine thiol or lysine amine group on the antibody, and so is typically a thiol-reactive group such as a double bond (as in maleimide) or a leaving group such as a chloro, bromo, iodo, or an R-sulfanyl group, or an amine-reactive group such as a carboxyl group; while the antibody conjugation reactive terminus of the linker is typically a site that is capable of conjugation to the cytotoxin through formation of an amide bond with a basic amine or carboxyl group on the cytotoxin, and so is typically a carboxyl or basic amine group. When the term “linker” is used in describing the linker in conjugated form, one or both of the reactive termini will be absent (such as reactive moiety Z, having been converted to chemical moiety Z) or incomplete (such as being only the carbonyl of the carboxylic acid) because of the formation of the bonds between the linker and/or the cytotoxin, and between the linker and/or the antibody or antigen-binding fragment thereof. Such conjugation reactions are described further herein below.

In some embodiments, the linker is cleavable under intracellular conditions, such that cleavage of the linker releases the drug unit from the antibody in the intracellular environment. In yet other embodiments, the linker unit is not cleavable and the drug is released, for example, by antibody degradation. The linkers useful for the present ADCs are preferably stable extracellularly, prevent aggregation of ADC molecules and keep the ADC freely soluble in aqueous media and in a monomeric state. Before transport or delivery into a cell, the ADC is preferably stable and remains intact, i.e. the antibody remains linked to the drug moiety. The linkers are stable outside the target cell and may be cleaved at some efficacious rate inside the cell. An effective linker will: (i) maintain the specific binding properties of the antibody; (ii) allow intracellular delivery of the conjugate or drug moiety; (iii) remain stable and intact, i.e. not cleaved, until the conjugate has been delivered or transported to its targeted site; and (iv) maintain a cytotoxic, cell-killing effect or a cytostatic effect of the cytotoxic moiety. Stability of the ADC may be measured by standard analytical techniques such as mass spectroscopy, HPLC, and the separation/analysis technique LC/MS. Covalent attachment of the antibody and the drug moiety requires the linker to have two reactive functional groups, i.e. bivalency in a reactive sense. Bivalent linker reagents which are useful to attach two or more functional or biologically active moieties, such as peptides, nucleic acids, drugs, toxins, antibodies, haptens, and reporter groups are known, and methods have been described their resulting conjugates (Hermanson, G. T. (1996) Bioconjugate Techniques; Academic Press: New York, p. 234-242).

Linkers include those that may be cleaved, for instance, by enzymatic hydrolysis, photolysis, hydrolysis under acidic conditions, hydrolysis under basic conditions, oxidation, disulfide reduction, nucleophilic cleavage, or organometallic cleavage (see, for example, Leriche et al., Bioorg. Med. Chem., 20:571-582, 2012, the disclosure of which is incorporated herein by reference as it pertains to linkers suitable for covalent conjugation).

Linkers hydrolyzable under acidic conditions include, for example, hydrazones, semicarbazones, thiosemicarbazones, cis-aconitic amides, orthoesters, acetals, ketals, or the like. (See, e.g., U.S. Pat. Nos. 5,122,368; 5,824,805; 5,622,929; Dubowchik and Walker, 1999, Pharm. Therapeutics 83:67-123; Neville et al., 1989, Biol. Chem. 264:14653-14661, the disclosure of each of which is incorporated herein by reference in its entirety as it pertains to linkers suitable for covalent conjugation. Such linkers are relatively stable under neutral pH conditions, such as those in the blood, but are unstable at below pH 5.5 or 5.0, the approximate pH of the lysosome.

Linkers cleavable under reducing conditions include, for example, a disulfide. A variety of disulfide linkers are known in the art, including, for example, those that can be formed using SATA (N-succinimidyl-S-acetylthioacetate), SPDP (N-succinimidyl-3-(2-pyridyldithio)propionate), SPDB (N-succinimidyl-3-(2-pyridyldithio)butyrate) and SMPT (N-succinimidyl-oxycarbonyl-alpha-methyl-alpha-(2-pyridyl-dithio)toluene), SPDB and SMPT (See, e.g., Thorpe et al., 1987, Cancer Res. 47:5924-5931; Wawrzynczak et al., In Immunoconjugates: Antibody Conjugates in Radioimagery and Therapy of Cancer (C. W. Vogel ed., Oxford U. Press, 1987. See also U.S. Pat. No. 4,880,935, the disclosure of each of which is incorporated herein by reference in its entirety as it pertains to linkers suitable for covalent conjugation.

Additional linkers include the non-cleavable maleimidocaproyl linkers, which are particularly useful for the conjugation of microtubule-disrupting agents such as auristatins, are described by Doronina et al., Bioconjugate Chem. 17:14-24, 2006, the disclosure of which is incorporated herein by reference as it pertains to linkers for chemical conjugation. Additional linkers suitable for the synthesis of drug-antibody and drug-ligand conjugates as described herein include those capable of releasing a cytotoxin by a 1,6-elimination process, (a “self-immolative” group), such as p-aminobenzyl alcohol (PABC), 6-maleimidohexanoic acid, pH-sensitive carbonates, and other reagents described in Jain et al., Pharm. Res. 32:3526-3540, 2015, the disclosure of which is incorporated herein by reference in its entirety.

In some embodiments, the linker includes a self-immolative group such as the afore-mentioned PAB or PABC (para-aminobenzyloxycarbonyl), which are disclosed in, for example, Carl et al., J. Med. Chem. (1981) 24:479-480; Chakravarty et al (1983) J. Med. Chem. 26:638-644; U.S. Pat. No. 6,214,345; US20030130189; US20030096743; U.S. Pat. No. 6,759,509; US20040052793; U.S. Pat. Nos. 6,218,519; 6,835,807; 6,268,488; US20040018194; WO98/13059; US20040052793; U.S. Pat. No. 6,677,435; 5,621,002; US20040121940; WO2004/032828). Other such chemical moieties capable of this process (“self-immolative linkers”) include methylene carbamates and heteroaryl groups such as aminothiazoles, aminoimidazoles, aminopyrimidines, and the like. Linkers containing such heterocyclic self-immolative groups are disclosed in, for example, U.S. Patent Publication Nos. 20160303254 and 20150079114, and U.S. Pat. No. 7,754,681; Hay et al. (1999) Bioorg. Med. Chem. Lett. 9:2237; US 2005/0256030; de Groot et al (2001) J. Org. Chem. 66:8815-8830; and U.S. Pat. No. 7,223,837.

Linkers susceptible to enzymatic hydrolysis can be, e.g., a peptide-containing linker that is cleaved by an intracellular peptidase or protease enzyme, including, but not limited to, a lysosomal or endosomal protease. One advantage of using intracellular proteolytic release of the therapeutic agent is that the agent is typically attenuated when conjugated and the serum stabilities of the conjugates are typically high. In some embodiments, the peptidyl linker is at least two amino acids long or at least three amino acids long. Exemplary amino acid linkers include a dipeptide, a tripeptide, a tetrapeptide or a pentapeptide. Examples of suitable peptides include those containing amino acids such as Valine, Alanine, Citrulline (Cit), Phenylalanine, Lysine, Leucine, and Glycine. Amino acid residues which comprise an amino acid linker component include those occurring naturally, as well as minor amino acids and non-naturally occurring amino acid analogs, such as citrulline. Exemplary dipeptides include valine-citrulline (vc or val-cit) and alanine-phenylalanine (af or ala-phe). Exemplary tripeptides include glycine-valine-citrulline (gly-val-cit) and glycine-glycine-glycine (gly-gly-gly). In some embodiments, the linker includes a dipeptide such as Val-Cit, Ala-Val, or Phe-Lys, Val-Lys, Ala-Lys, Phe-Cit, Leu-Cit, Ile-Cit, Phe-Arg, or Trp-Cit. Linkers containing dipeptides such as Val-Cit or Phe-Lys are disclosed in, for example, U.S. Pat. No. 6,214,345, the disclosure of which is incorporated herein by reference in its entirety as it pertains to linkers suitable for covalent conjugation. In some embodiments, the linker includes a dipeptide selected from Val-Ala and Val-Cit. In some embodiments, a dipeptide is used in combination with a self-immolative linker.

Linkers suitable for use herein further may include one or more groups selected from C₁-C₆ alkylene, C₁-C₆ heteroalkylene, C₂-C₆ alkenylene, C₂-C₆ heteroalkenylene, C₂-C₆ alkynylene, C₂-C₆ heteroalkynylene, C₃-C₆ cycloalkylene, heterocycloalkylene, arylene, heteroarylene, and combinations thereof, each of which may be optionally substituted. Non-limiting examples of such groups include (CH₂)ₙ , (CH₂CH₂O)ₙ, and —(C═O)(CH₂)ₙ— units, wherein n is an integer from 1-6, independently selected for each occasion.

In some embodiments, the linker may include one or more of a hydrazine, a disulfide, a thioether, a dipeptide, a p-aminobenzyl (PAB) group, a heterocyclic self-immolative group, an optionally substituted C₁-C₆ alkyl, an optionally substituted C₁-C₆ heteroalkyl, an optionally substituted C₂-C₆ alkenyl, an optionally substituted C₂-C₆ heteroalkenyl, an optionally substituted C₂-C₆ alkynyl, an optionally substituted C₂-C₆ heteroalkynyl, an optionally substituted C₃-C₆ cycloalkyl, an optionally substituted heterocycloalkyl, an optionally substituted aryl, an optionally substituted heteroaryl, acyl, —(C═O)—, or —(CH₂CH₂O)ₙ— group, wherein n is an integer from 1-6. One of skill in the art will recognize that one or more of the groups listed may be present in the form of a bivalent (diradical) species, e.g., C₁-C₆ alkylene and the like.

In some embodiments, the linker includes a p-aminobenzyl group (PAB). In one embodiment, the p-aminobenzyl group is disposed between the cytotoxic drug and a protease cleavage site in the linker. In one embodiment, the p-aminobenzyl group is part of a p-aminobenzyloxycarbonyl unit. In one embodiment, the p-aminobenzyl group is part of a p-aminobenzylamido unit.

In some embodiments, the linker comprises PAB, Val-Cit-PAB, Val-Ala-PAB, Val-Lys(Ac)-PAB, Phe-Lys-PAB, Phe-Lys(Ac)-PAB, D-Val-Leu-Lys, Gly-Gly-Arg, Ala-Ala-Asn-PAB, or Ala-PAB.

In some embodiments, the linker comprises a combination of one or more of a peptide, oligosaccharide, —(CH₂)ₙ—, —(CH₂CH₂O)ₙ—, PAB, Val-Cit-PAB, Val-Ala-PAB, Val-Lys(Ac)-PAB, Phe-Lys-PAB, Phe-Lys(Ac)-PAB, D-Val-Leu-Lys, Gly-Gly-Arg, Ala-Ala-Asn-PAB, or Ala-PAB.

In some embodiments, the linker comprises a —(C═O)(CH₂)ₙ— unit, wherein n is an integer from 1-6.

In some embodiments, the linker comprises a —(CH₂)ₙ— unit, wherein n is an integer from 2 to 6.

In certain embodiments, the linker of the ADC is N-beta-maleimidopropyl-Val-Ala-para-aminobenzyl (BMP-Val-Ala-PAB).

Linkers that can be used to conjugate an antibody, antigen-binding fragment thereof, or ligand to a cytotoxic agent include those that are covalently bound to the cytotoxic agent on one end of the linker and, on the other end of the linker, contain a chemical moiety formed from a coupling reaction between a reactive substituent present on the linker and a reactive substituent present within the antibody, antigen-binding fragment thereof, or ligand that binds CD134 or CD278. Reactive substituents that may be present within an antibody, antigen-binding fragment thereof, or ligand that binds CD134 or CD278 include, without limitation, hydroxyl moieties of serine, threonine, and tyrosine residues; amino moieties of lysine residues; carboxyl moieties of aspartic acid and glutamic acid residues; and thiol moieties of cysteine residues, as well as propargyl, azido, haloaryl (e.g., fluoroaryl), haloheteroaryl (e.g., fluoroheteroaryl), haloalkyl, and haloheteroalkyl moieties of non-naturally occurring amino acids. Examples of linkers useful for the synthesis of drug-antibody conjugates conjugates include those that contain electrophiles, such as Michael acceptors (e.g., maleimides), activated esters, electron-deficient carbonyl compounds, and aldehydes, among others, suitable for reaction with nucleophilic substituents present within antibodies or antigen-binding fragments, such as amine and thiol moieties. For instance, linkers suitable for the synthesis of drug-antibody conjugates include, without limitation, succinimidyl 4-(N-maleimidomethyl)-cyclohexane-L-carboxylate (SMCC), N-succinimidyl iodoacetate (SIA), sulfo-SMCC, m-maleimidobenzoyl-N-hydroxysuccinimidyl ester (MBS), sulfo-MBS, and succinimidyl iodoacetate, among others described, for instance, Liu et al., 18:690-697, 1979, the disclosure of which is incorporated herein by reference as it pertains to linkers for chemical conjugation.

It will be recognized by one of skill in the art that any one or more of the chemical groups, moieties and features disclosed herein may be combined in multiple ways to form linkers useful for conjugation of the antibodies and cytotoxins as disclosed herein. Further linkers useful in conjunction with the compositions and methods described herein, are described, for example, in U.S. Patent Application Publication No. 2015/0218220, the disclosure of which is incorporated herein by reference in its entirety.

Linkers useful in conjunction with the antibody-drug and ligand-conjugates described herein include, without limitation, linkers containing chemical moieties formed by coupling reactions as depicted in Table 1, below. Curved lines designate points of attachment to the antibody, antigen-binding fragment, or ligand and the cytotoxic molecule, respectively.

TABLE 1
Exemplary chemical moieties formed by coupling reactions in the formation of antibody-
drug conjugates

| Exemplary | |
|---|---|
| Coupling Reactions | Chemical Moiety Z Formed by Coupling Reactions |
| [3 + 2] Cycloaddition | 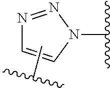 |
| [3 + 2] Cycloaddition | 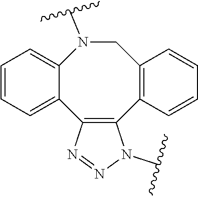 [SMILES: *N1Cc2ccccc2-c2c(nnn2*)-c2ccccc21] |
| [3 + 2] Cycloaddition, Esterification | 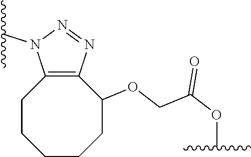 [SMILES: *OC(=O)COC1CCCCCc2c1nnn2*] |
| [3 + 2] Cycloaddition, Esterification | 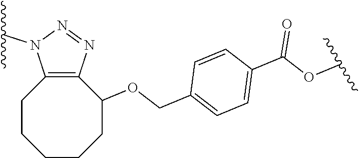 [SMILES: *OC(=O)c1ccc(COC2CCCCCc3c2nnn3*)cc1] |
| [3 + 2] Cycloaddition, Esterification | 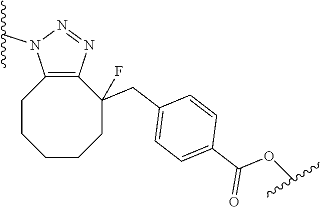 [SMILES: *OC(=O)c1ccc(CC2(F)CCCCCc3c2nnn3*)cc1] |
| [3 + 2] Cycloaddition, Esterification | 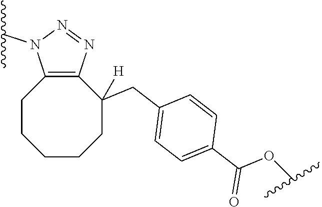 [SMILES: *OC(=O)c1ccc(CC2CCCCCc3c2nnn3*)cc1] |
| [3 + 2] Cycloaddition, Esterification | 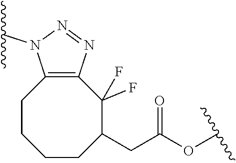 [SMILES: *OC(=O)CC1CCCCc2c(nnn2*)C1(F)F] |
| [3 + 2] Cycloaddition, Esterification | 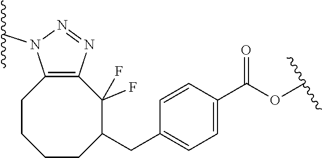 [SMILES: *OC(=O)c1ccc(CC2CCCCc3c(nnn3*)C2(F)F)cc1] |
| [3 + 2] Cycloaddition, Esterification | 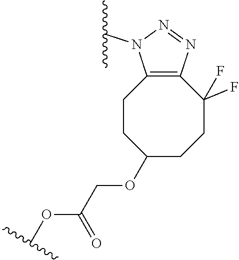 [SMILES: *OC(=O)COC1CCc2c(nnn2*)C(F)(F)CC1] |
| [3 + 2] Cycloaddition, Esterification | 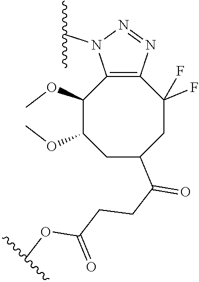 [SMILES: *OC(=O)CCC(=O)C1C[C@H](OC)[C@@H](OC)c2c(nnn2*)C(F)(F)C1] |
| [3 + 2] Cycloaddition, Esterification | 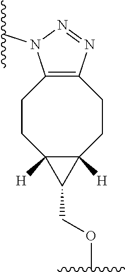 [SMILES: *OC[C@@H]1[C@@H]2CCc3c(nnn3*)CC[C@@H]21] |
| [3 + 2] Cycloaddition, Esterification | 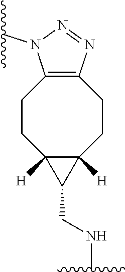 [SMILES: *NC[C@@H]1[C@@H]2CCc3c(nnn3*)CC[C@@H]21] |
| [3 + 2] Cycloaddition, Esterification | 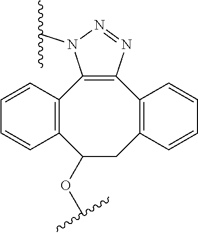 [SMILES: *OC1Cc2ccccc2-c2nnn(*)c2-c2ccccc21] |
| [3 + 2] Cycloaddition, Etherification | 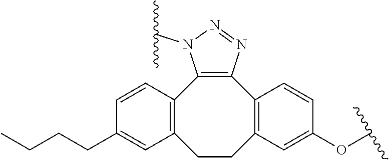 [SMILES: *Oc1ccc2c(c1)CCc1cc(CCCC)ccc1-c1c-2nnn1*] |
| [3 + 2] Cycloaddition | 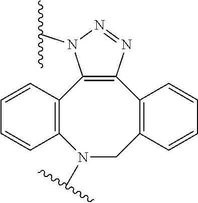 [SMILES: *N1Cc2ccccc2-c2nnn(*)c2-c2ccccc21] |
| Michael addition | 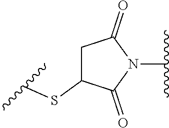 [SMILES: *SC1CC(=O)N(*)C1=O] |
| Michael addition | 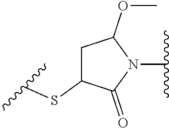 [SMILES: *SC1CC(OC)N(*)C1=O] |
| Imine condensation, Amidation | 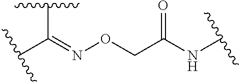 [SMILES: *NC(=O)CON=C(*)*] |
| Imine condensation | 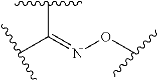 [SMILES: *ON=C(*)*] |
| Disulfide formation | 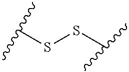 [SMILES: *SS*] |
| Thiol alkylation | 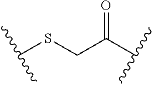 [SMILES: *SCC(*)=O] |
| Condensation, Michael addition | 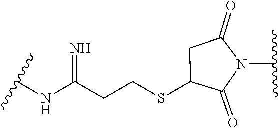 [SMILES: *NC(=N)CCSC1CC(=O)N(*)C1=O] |

One of skill in the art will recognize that a reactive substituent Z attached to the linker and a reactive substituent on the antibody or antigen-binding fragment thereof, are engaged in the covalent coupling reaction to produce the chemical moiety Z, and will recognize the reactive substituent Z. Therefore, antibody-drug conjugates useful in conjunction with the methods described herein may be formed by the reaction of an antibody, or antigen-binding fragment thereof, with a linker or cytotoxin-linker conjugate, as described herein, the linker or cytotoxin-linker conjugate including a reactive substituent Z, suitable for reaction with a reactive substituent on the antibody, or antigen-binding fragment thereof, to form the chemical moiety Z.

As depicted in Table 1, examples of suitably reactive substituents on the linker and antibody or antigen-binding fragment thereof include a nucleophile/electrophile pair (e.g., a thiol/haloalkyl pair, an amine/carbonyl pair, or a thiol/α,β-unsaturated carbonyl pair, and the like), a diene/dienophile pair (e.g., an azide/alkyne pair, or a diene/α,β-unsaturated carbonyl pair, among others), and the like. Coupling reactions between the reactive substituents to form the chemical moiety Z include, without limitation, thiol alkylation, hydroxyl alkylation, amine alkylation, amine or hydroxylamine condensation, hydrazine formation, amidation, esterification, disulfide formation, cycloaddition (e.g., [4+2] Diels-Alder cycloaddition, [3+2] Huisgen cycloaddition, among others), nucleophilic aromatic substitution, electrophilic aromatic substitution, and other reactive modalities known in the art or described herein. Preferably, the linker contains an electrophilic functional group for reaction with a nucleophilic functional group on the antibody, or antigen-binding fragment thereof.

Reactive substituents that may be present within an antibody, or antigen-binding fragment thereof, as disclosed herein include, without limitation, nucleophilic groups such as (i) N-terminal amine groups, (ii) side chain amine groups, e.g. lysine, (iii) side chain thiol groups, e.g. cysteine, and (iv) sugar hydroxyl or amino groups where the antibody is glycosylated. Reactive substituents that may be present within an antibody, or antigen-binding fragment thereof, as disclosed herein include, without limitation, hydroxyl moieties of serine, threonine, and tyrosine residues; amino moieties of lysine residues; carboxyl moieties of aspartic acid and glutamic acid residues; and thiol moieties of cysteine residues, as well as propargyl, azido, haloaryl (e.g., fluoroaryl), haloheteroaryl (e.g., fluoroheteroaryl), haloalkyl, and haloheteroalkyl moieties of non-naturally occurring amino acids. In some embodiments, the reactive substituents present within an antibody, or antigen-binding fragment thereof as disclosed herein include, are amine or thiol moieties. Certain antibodies have reducible interchain disulfides, i.e. cysteine bridges. Antibodies may be made reactive for conjugation with linker reagents by treatment with a reducing agent such as DTT (dithiothreitol). Each cysteine bridge will thus form, theoretically, two reactive thiol nucleophiles. Additional nucleophilic groups can be introduced into antibodies through the reaction of lysines with 2-iminothiolane (Traut's reagent) resulting in conversion of an amine into a thiol. Reactive thiol groups may be introduced into the antibody (or fragment thereof) by introducing one, two, three, four, or more cysteine residues (e.g., preparing mutant antibodies comprising one or more non-native cysteine amino acid residues). U.S. Pat. No. 7,521,541 teaches engineering antibodies by introduction of reactive cysteine amino acids.

In some embodiments, the reactive moiety Z attached to the linker is a nucleophilic group which is reactive with an electrophilic group present on an antibody. Useful electrophilic groups on an antibody include, but are not limited to, aldehyde and ketone carbonyl groups. The heteroatom of a nucleophilic group can react with an electrophilic group on an antibody and form a covalent bond to the antibody. Useful nucleophilic groups include, but are not limited to, hydrazide, oxime, amino, hydroxyl, hydrazine, thiosemicarbazone, hydrazine carbon/late, and arylhydrazide. In some embodiments, Z is the product of a reaction between reactive nucleophilic substituents present within the antibodies, or antigen-binding fragments thereof, such as amine and thiol moieties, and a reactive electrophilic substituent Z. For instance, Z may be a Michael acceptor (e.g., maleimide), activated ester, electron-deficient carbonyl compound, or an aldehyde, among others.

In some embodiments, the ADC comprises an anti-CD134 or anti-CD278 antibody conjugated to an amatoxin of any of formulae III, IIIA, or IIIB as disclosed herein via a linker and a chemical moiety Z, to form an amatoxin-linker conjugate of any of formulae I, IA, IB, II, IIA, or IIB as disclosed herein. In some embodiments, the linker includes a dipeptide. In some embodiments, the linker includes a dipeptide selected from Val-Ala and Val-Cit. In some embodiments, the linker includes a para-aminobenzyl group (PAB). In some embodiments, the linker includes the moiety PAB-Cit-Val. In some embodiments, the linker includes the moiety PAB-Ala-Val. In some embodiments, the linker includes a —((C═O)(CH₂)ₙ— unit, wherein n is an integer from 1-6. In some embodiments, the linker is -PAB-Cit-Val-((C═O)(CH₂)ₙ—.

In some embodiments, the linker includes a —(CH₂)ₙ— unit, where n is an integer from 2-6. In some embodiments, the linker is -PAB-Cit-Val-((C═O)(CH₂)ₙ—. In some embodiments, the linker is -PAB-Ala-Val-((C═O)(CH₂)ₙ—. In some embodiments, the linker is —(CH₂)ₙ—. In some embodiments, the linker is —((CH₂)ₙ— , wherein n is 6.

In some embodiments, the chemical moiety Z is selected from Table 1. In some embodiments, the chemical moiety Z is

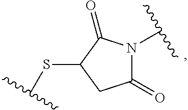

where S is a sulfur atom which represents the reactive substituent present within an antibody, or antigen-binding fragment thereof, that binds CD134 or CD278 (e.g., from the —SH group of a cysteine residue).

In some embodiments, the linker L and the chemical moiety Z, taken together as L-Z, is

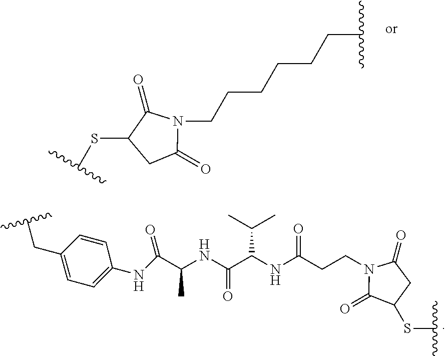

One of skill in the art will recognize the linker-reactive substituent group structure, prior to conjugation with the antibody or antigen binding fragment thereof, includes a maleimide as the group Z. The foregoing linker moieties and amatoxin-linker conjugates, among others useful in conjunction with the compositions and methods described herein, are described, for example, in U.S. Patent Application Publication No. 2015/0218220 and Patent Application Publication No. WO2017/149077, the disclosure of each of which is incorporated herein by reference in its entirety.

In some embodiments, the linker-reactive substituent group structure, prior to conjugation with the antibody or antigen binding fragment thereof, is:

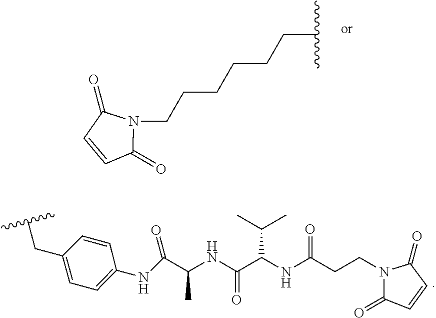

### Preparation of Antibody-Drug Conjugates

In the ADCs of formulae I, IA, IB, II, IIA, and IIB as disclosed herein, an antibody or antigen binding fragment thereof is conjugated to one or more cytotoxic drug moieties (D), e.g. about 1 to about 20 drug moieties per antibody, through a linker L and a chemical moiety Z as disclosed herein. The ADCs of the present disclosure may be prepared by several routes, employing organic chemistry reactions, conditions, and reagents known to those skilled in the art, including: (1) reaction of a reactive substituent of an antibody or antigen binding fragment thereof with a bivalent linker reagent to form Ab-Z-L as described herein above, followed by reaction with a drug moiety D; or (2) reaction of a reactive substituent of a drug moiety with a bivalent linker reagent to form D-L-Z, followed by reaction with a reactive substituent of an antibody or antigen binding fragment thereof as described herein above to form an ADC of formula D-L-Z-Ab, such as Am-Z-L-Ab. Additional methods for preparing ADC are described herein.

In another aspect, the antibody or antigen binding fragment thereof has one or more lysine residues that can be chemically modified to introduce one or more sulfhydryl groups. The ADC is then formed by conjugation through the sulfhydryl group's sulfur atom as described herein above. The reagents that can be used to modify lysine include, but are not limited to, N-succinimidyl S-acetylthioacetate (SATA) and 2-Iminothiolane hydrochloride (Traut's Reagent). In another aspect, the antibody or antigen binding fragment thereof can have one or more carbohydrate groups that can be chemically modified to have one or more sulfhydryl groups. The ADC is then formed by conjugation through the sulfhydryl group's sulfur atom as described herein above.

In yet another aspect, the antibody can have one or more carbohydrate groups that can be oxidized to provide an aldehyde (—CHO) group (see, for e.g., Laguzza, et al., J. Med. Chem. 1989, 32(3), 548-55). The ADC is then formed by conjugation through the corresponding aldehyde as described herein above. Other protocols for the modification of proteins for the attachment or association of cytotoxins are described in Coligan et al., Current Protocols in Protein Science, vol. 2, John Wiley & Sons (2002), incorporated herein by reference.

Methods for the conjugation of linker-drug moieties to cell-targeted proteins such as antibodies, immunoglobulins or fragments thereof are found, for example, in U.S. Pat. Nos. 5,208,020; 6,441,163; WO2005037992; WO2005081711; and WO2006/034488, all of which are hereby expressly incorporated by reference in their entirety.

Alternatively, a fusion protein comprising the antibody and cytotoxic agent may be made, e.g., by recombinant techniques or peptide synthesis. The length of DNA may comprise respective regions encoding the two portions of the conjugate either adjacent one another or separated by a region encoding a linker peptide which does not destroy the desired properties of the conjugate.

### Antibody Pharmacokinetic Profile

In some embodiments, the antibody, antigen-binding fragment thereof, or drug-antibody conjugate has a defined serum half-life. Antibodies, antigen-binding fragments thereof, and conjugates useful in the methods herein include those that have a serum half-life, for example, from 1-24 hours. In some embodiments, the transplant is administered prior, at the same time or after the antibody, antigen-binding fragment thereof, drug-antibody conjugate, when the level of the circulating antibody is at a therapeutically effective level. Pharmacokinetic analysis by measurement of serum levels can be performed by assays known in the art.

### Routes of Administration and Dosing

Antibodies, antigen-binding fragments thereof, ADCs, and ligands described herein can be administered to a patient (e.g., a human patient suffering from or at risk for GVHD or an autoimmune disease) in a variety of dosage forms. For instance, antibodies, antigen-binding fragments thereof, ADCs, and ligands described herein can be administered to a patient patient suffering from or at risk for GVHD in the form of an aqueous solution, such as an aqueous solution containing one or more pharmaceutically acceptable excipients. Suitable pharmaceutically acceptable excipients for use with the compositions and methods described herein include viscosity-modifying agents. The aqueous solution may be sterilized using techniques known in the art.

Pharmaceutical formulations comprising an anti-CD134 ADC or anti-CD278 ADC as described herein are prepared by mixing such ADC with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

The antibodies, antigen-binding fragments, ADCs, and ligands described herein may be administered by a variety of routes, such as orally, transdermally, subcutaneously, intranasally, intravenously, intramuscularly, intraocularly, or parenterally. The most suitable route for administration in any given case will depend on the particular antibody, antigen-binding fragment administered, or ADC, the patient, pharmaceutical formulation methods, administration methods (e.g., administration time and administration route), the patient's age, body weight, sex, severity of the diseases being treated, the patient's diet, and the patient's excretion rate.

The effective dose of an antibody, antigen-binding fragment thereof, ADC, or ligand described herein can range, for example from about 0.001 to about 100 mg/kg of body weight per single (e.g., bolus) administration, multiple administrations, or continuous administration, or to achieve an optimal serum concentration (e.g., a serum concentration of 0.0001-5000 μg/mL) of the antibody, antigen-binding fragment thereof, ADC, or soluble ligand. The dose may be administered one or more times (e.g., 2-10 times) per day, week, or month to a subject (e.g., a human) suffering from or at risk for GVHD or an autoimmune disease. The antibody, antigen-binding fragment thereof, ADC, or ligand can be administered in an amount sufficient to reduce the quantity of host-reactive T cells, for example, by 10%, 20%, 30%, 40%, 50%, 60%, 70%, 80%, 90%, 95%, or more prior to hematopoietic stem cell transplant.

### Methods of Treatment

The compositions and methods described herein may be used to deplete activated T cells that are associated with graft failure, particularly allograft rejection, and autoimmune diseases in order to achieve transplant tolerance. The compositions and methods described herein are particularly useful for preventing and treating GVHD and/or autoimmune diseases. The compositions and methods described herein are also useful for preventing or treating host versus graft disease (HvGD). The methods and compositions disclosed herein are also useful in reducing the risk of transplant failure in a human patient receiving an allogenic transplant. The preferred subject is human. The amount of antibody, antibody-drug conjugate, or ligand-drug conjugate administered should be sufficient to deplete cells, e.g., activated T cells, that promote GVHD or autoimmune disease. The determination of a therapeutically effective dose is within the capability of practitioners in this art, however, as an example, in embodiments of the method described herein utilizing systemic administration of an antibody for the treatment of GHVD or autoimmune disease, an effective human dose will be in the range of 0.1-150 mg/kg (e.g., 5 mg/kg, 10 mg/kg, 25 mg/kg, 50 mg/kg, 75 mg/kg, 100 mg/kg, 150 mg/kg etc.). The route of administration may affect the recommended dose. Repeated systemic doses are contemplated in order to maintain an effective level, e.g., to attenuate or inhibit GVHD or autoimmune disease, depending on the mode of administration adopted.

The antibody, antibody-drug conjugate, or ligand-drug conjugate can be administered to the human patient in need prior to, concomitantly with, or after transplantation of cells or a solid organ to the patient. In one embodiment, an anti-CD134 ADC or anti-CD278 ADC is administered to the human patient in need thereof prior to (e.g., about 3 days before, about 2 days before, about 12 hours before) transplantation of cells or a solid organ. In one embodiment, an anti-CD134 ADC or anti-CD278 ADC is administered to the human patient in need thereof after (e.g., about 1 days after, about 2 days after, about 3 days after, or about 4 days after) transplantation of cells or a solid organ. In certain embodiments, the ADCs described herein are administered to the patient prior to, concomitantly with, and/or following transplantation. A single dose of an anti-CD134 ADC or anti-CD278 ADC may be administered to the human patient either prior to, concomitantly with, or after transplantation of cells or an organ, where such single dose is sufficient to treat or prevent GVHD or graft failure.

Anti-CD134 ADCs or anti-CD278 ADCs may be used as an alternative to traditional agents (e.g., chemotherapy and/or radiation) used to promote acceptance of a transplant, including an allogeneic transplant. Traditional agents generally reduce a patient's immune response in order to promote engraftment and acceptance of the transplanted cells or organ. The methods and compositions described herein provide a more selective therapy that allows much of the patient's immune system to remain intact, while targeting and depleting CD134 expressing activated T cells or CD278 expressing activated T cells. Thus, the ability of anti-CD134 ADCs or anti-CD278 ADCs disclosed herein to selectively deplete activated T cells provides an advantageous therapy over traditional therapy in the context of transplantation given that, in particular, allo-activated immune cells can be targeted and depleted in order to achieve successful transplantation of cells or a solid organ.

The methods and compositions disclosed herein may be used to prevent or treat graft failure. Graft failure or graft rejection, including failure after allogeneic hematopoietic stem cell transplantation, may be manifested generally as either lack of initial engraftment of donor cells, or loss of donor cells after initial engraftment (for review see Mattsson et al. (2008) Biol Blood Marrow Transplant. 14(Suppl 1): 165-170). Compositions and methods disclosed herein may be used to deplete CD134 or CD278 expressing activated T cells in a graft or transplantation scenario where graft failure is of concern, e.g., where the human patient is at risk of developing graft failure following transplantation of a solid organ or cells, particularly where the transplanted cells or organ is allogeneic.

In one embodiment, the anti-CD134 or anti-CD278 antibody, antibody-drug conjugate, or ligand-drug conjugate is used to deplete CD134 or CD278 expressing donor cells, e.g., activated T cells expressing CD134 or CD278, by contacting the cells, graft or solid organ with the anti-CD134 or anti-CD278 antibody, antibody-drug conjugate, or ligand-drug conjugate prior to transplantation of the cells, graft or organ to a human patient. In one embodiment, the cells, graft or organ are allogeneic.

The risk of GVHD remains high following transplantation with current therapies. The methods and compositions disclosed herein may be used to inhibit graft versus host disease (GVHD) in a human patient. The anti-CD134 ADCs or anti-CD278 ADCs may be used to selectively target activated T cells in a patient who will be receiving a transplant, such as a stem cell transplant. Anti-CD134 ADCs or anti-CD278 ADCs, as described herein, may also be used to reduce the risk of GVHD by targeting and depleting CD134 positive or CD278 positive cells in a human patient who is going to be or has already received a transplant, such as but not limited to, an HSC transplant. In certain embodiments, the compositions and methods disclosed herein are for treating GvHD prior to appearance of symptoms of GVHD in a patient following a transplantation therapy, e.g., allogeneic HSCs.

The methods described herein are also useful for preventing host versus graft (HvG) reactions. An anti-CD134-ADC or anti-CD278-ADC can also be used as an immunosuppressant to prevent host versus graft (HvG) reactions thereby preventing or reducing the risk of allogeneic graft failure. Use of an anti-CD134 or anti-CD278 ADC in a patient at risk for a HvG reaction would enable engraftment of donor cells with a greater degree of HLA-mismatch. Additional uses include tolerance induction in solid organ transplant, where host versus graft reactions are prevented or dampened by the CD134-ADC or CD278-ADC. These would include solid organ transplants done with or without hematopoietic stem cell transplants, including xeno-transplants where the organ is non-human in origin and/or genetically modified.

In one embodiment, an anti-CD134-ADC or anti-CD278-ADC is used to prevent graft versus graft (GvG) in the context of allogeneic transplants where two donors are used. Examples include the use of 2 cord blood stem cell donors in adults and pediatric patients. Prevention of GvG would enable more rapid hematopoietic (e.g. neutrophil and platelet) reconstitution post-transplant as both stem cell sources would successfully engraft.

In some embodiments, the transplant is allogeneic. In some embodiments, the transplant is autologous.

In some embodiments, the transplant is a bone marrow transplant, a peripheral blood transplant, or a cord blood transplant.

In some embodiments, the transplant includes hematopoietic cells (e.g., hematopoietic stem cells).

In any of the embodiments described herein, the transplant maybe any solid organ or skin transplant. In some embodiments, the transplant is selected from the group consisting of kidney transplant, heart transplant, liver transplant, pancreas transplant, lung transplant, intestine transplant and skin transplant.

The methods described herein are useful for treating multiple sclerosis (MS). MS is a devastating autoimmune inflammatory disease of the central nervous system. It is well accepted that the damage in the central nervous system (CNS) results from an autoimmune attack against (auto) antigens within the myelin sheath. The mechanisms responsible for tissue damage in MS involve the activation of self-reactive T cells, which attack proteins in the myelin sheath. It is common for individuals to experience the first signs between the ages of 15 and 50. Affected individuals encounter bouts of inflammatory demyelination producing the classic course of the disease of exacerbation—remittance.

The methods described herein are also useful for treating human systemic lupus (SLE). SLE, or lupus, is a systemic chronic autoimmune disease characterized by autoantibody production against self-antigens. Autoreactive B cells are driven by self-antigen, including antibodies to double stranded DNA, to nuclear protein antigens and to ribonucleoproteins. The factors that promote the loss of B cell tolerance and drive autoantibody production are unknown. Systemic lupus can affect almost any organ or system of the body. Systemic lupus may include periods in which few, if any, symptoms are evident (“remission”) and other times when the disease becomes more active (“flare”).

The methods described herein are also useful for treating rheumatoid arthritis (RA). RA is a systemic autoimmune disease which initially attacks the synovium, a connective tissue membrane that lines the cavity between joints and secretes a lubricating fluid. Although the cause of RA is unknown, infectious, genetic, and hormonal factors may contribute to the RA. RA is associated with abnormal immunity, as the joints of patients suffering from RA are severely infiltrated with leukocytes, such as macrophages and dendritic cells, and T and B cells. The disease can occur at any age, but the peak incidence of disease onset is between the ages of 25 and 55. The incidence increases with age. The onset of the disease is usually gradual, with fatigue, morning stiffness lasting more than one hour, diffuse muscular aches, loss of appetite, and weakness. Eventually, joint pain appears, with warmth, swelling, tenderness, and stiffness of the joint after inactivity.

The methods described herein are also useful for treating inflammatory bowel disease (IBD). Manifestations of IBD include ulcerative colitis, Crohn's disease, lymphocytic colitis, and collagenous colitis. IBD is a spontaneously relapsing, immunologically mediated disorder of the gastrointestinal tract, characterized by uncontrolled inflammation and persistent activation of the mucosal immune system. CD4 T cells are believed to play a critical role in the pathogenesis of human IBD, due to their influx into the inflamed mucosa.

The methods described herein are particularly useful for treating psoriasis. Psoriasis is a chronic inflammatory skin disease characterized by red, scaly, raised plaques. Psoriasis is mediated by T cells and associated elevation in cytokine levels leading to increased cell division and aberrant differentiation. Psoriasis is a chronic, recurrent skin condition with varying degrees of severity and is also associated with serious co-morbidities, including psoriatic arthritis, depression, malignancy, metabolic syndrome, cardiovascular morbidity and mortality and autoimmune diseases, such as inflammatory bowel disease (IBD).

The methods described herein are also useful for treating Type 1 diabetes mellitus (Type 1 diabetes). Type 1 diabetes is a metabolic disorder in humans that include juvenile onset patients that are not over-weight relative to their age and height, with rapid onset of the disease at an early age, often before 30, although Type 1 diabetes can occur at any age. Type 1 diabetes is considered to be a disease of autoimmune etiology. CD4 and CD8 T cells have been implicated as causative agents for damage to beta cells (insulin producing cells).

The methods described herein are also useful for treating other autoimmune diseases including, but not limited to, acute disseminated encephalomyelitis (ADEM), Addison's disease, alopecia universalis, ankylosing spondylitisis, antiphospholipid antibody syndrome (APS), aplastic anemia, autoimmune hemolytic anemia, autoimmune hepatitis, autoimmune inner ear disease (AIED), autoimmune lymphoproliferative syndrome (ALPS), autoimmune oophoritis, Balo disease, Behcet's disease, bullous pemphigoid, cardiomyopathy, Chagas' disease, chronic fatigue immune dysfunction syndrome (CFIDS), chronic inflammatory demyelinating polyneuropathy, Crohn's disease, cicatrical pemphigoid, coeliac sprue-dermatitis herpetiformis, cold agglutinin disease, CREST syndrome, Degos disease, discoid lupus, dysautonomia, endometriosis, essential mixed cryoglobulinemia, fibromyalgia-fibromyositis, Goodpasture's syndrome, Grave's disease, Guillain-Barre syndrome (GBS), Hashimoto's thyroiditis, Hidradenitis suppurativa, idiopathic and/or acute thrombocytopenic purpura, idiopathic pulmonary fibrosis, IgA neuropathy, interstitial cystitis, juvenile arthritis, Kawasaki's disease, lichen planus, Lyme disease, Meniere disease, mixed connective tissue disease (MCTD), myasthenia gravis, neuromyotonia, opsoclonus myoclonus syndrome (OMS), optic neuritis, Ord's thyroiditis, pemphigus vulgaris, pernicious anemia, polychondritis, polymyositis and dermatomyositis, primary biliary cirrhosis, polyarteritis nodosa, polyglandular syndromes, polymyalgia rheumatica, primary agammaglobulinemia, Raynaud phenomenon, Reiter's syndrome, rheumatic fever, sarcoidosis, scleroderma, Sjogren's syndrome, stiff person syndrome, Takayasu's arteritis, temporal arteritis (also known as “giant cell arteritis”), ulcerative colitis, uveitis, vasculitis, vitiligo, vulvodynia (“vulvar vestibulitis”), and Wegener's granulomatosis.

The compositions and methods described herein can be used to treat a variety of disorders, including, without limitation, a non-malignant hemoglobinopathy (e.g., a hemoglobinopathy selected from the group consisting of sickle cell anemia, thalassemia, Fanconi anemia, and Wiskott-Aldrich syndrome). Additionally or alternatively, the compositions and methods described herein can be used to treat an immunodeficiency, such as a congenital immunodeficiency. Additionally or alternatively, the compositions and methods described herein can be used to treat an acquired immunodeficiency (e.g., an acquired immunodeficiency selected from the group consisting of HIV and AIDS). The compositions and methods described herein can be used to treat a metabolic disorder (e.g., a metabolic disorder selected from the group consisting of glycogen storage diseases, mucopolysaccharidoses, Gaucher's Disease, Hurlers Disease, sphingolipidoses, and metachromatic leukodystrophy). Additionally or alternatively, the compositions and methods described herein can be used to treat a malignancy, such as a hematologic cancer (e.g., leukemia, lymphoma, multiple myeloma and myelodysplastic syndrome), as well as other cancerous conditions, including neuroblastoma.

In one embodiment, the ADCs described herein are used to treat or prevent GVHD in a human patient being treated for leukemia, e.g., acute myelogenous leukemia, whereby the patient received an allogeneic transplant, e.g., an allogeneic transplant of hematopoietic stem cells (HSCs). The ADCs described here may also be used prior to the transplant to deplete T cells and promote acceptance of the allogeneic transplantation.

In one embodiment, the ADCs described herein are used to treat or prevent GVHD in a human patient being treated for a metabolic disorder, e.g, inherited metabolic disease, whereby the patient received an allogeneic transplant, e.g., an allogeneic transplant of cord blood cells. The ADCs described here may also be used prior to the transplant to deplete T cells and promote acceptance of the allogeneic cord blood cell transplantation.

Additional disorders that can be treated by administration of the compositions and methods described herein include adenosine deaminase deficiency and severe combined immunodeficiency, hyper immunoglobulin M syndrome, Chediak-Higashi disease, hereditary lymphohistiocytosis, osteopetrosis, osteogenesis imperfecta, storage diseases, thalassemia major, systemic sclerosis, systemic lupus erythematosus, multiple sclerosis, and juvenile rheumatoid arthritis.

According to the methods disclosed herein, an anti-CD134 or anti-CD278 antibody, antigen-binding fragment thereof, or ADC may be administered to a human patient in preparation for hematopoietic stem cell transplant therapy.

The anti-CD134 or anti-CD278 antibody, fragment thereof, ADC, or soluble ligand may be covalently conjugated to a toxin, such as a cytotoxic molecule described herein or known in the art, or an Fc domain. For instance, an anti-CD134 or anti-CD278 antibody, antigen-binding fragment thereof, ADC, or soluble ligand can be covalently conjugated to a cytotoxin, such as microtubule-binding agent, maytansine, a maytansinoid, an amatoxin, pseudomonas exotoxin A, deBouganin, diphtheria toxin, such as α-amanitin, saporin, an auristatin, an anthracycline, a calicheamicin, irinotecan, SN-38, a duocarmycin, a pyrrolobenzodiazepine, a pyrrolobenzodiazepine dimer, an indolinobenzodiazepine, and an indolinobenzodiazepine dimer, or a variant thereof.

This conjugation can be performed using covalent bond-forming techniques described herein or known in the art. The antibody, antigen-binding fragment thereof, or drug-antibody conjugate, or drug-ligand conjugate can subsequently be administered to the patient, for example, by intravenous administration, prior to transplantation of exogenous hematopoietic stem cells (such allogeneic hematopoietic stem cells) to the patient.

The anti-CD134 or anti-CD278 antibody, antigen-binding fragment thereof, drug-antibody conjugate, or drug-ligand conjugate can be administered in an amount sufficient to reduce the quantity of host-reactive T cells, for example, by 10%, 20%, 30%, 40%, 50%, 60%, 70%, 80%, 90%, 95%, or more prior, at the time of, or after the hematopoietic stem cell transplant therapy. The reduction in donor T cell count can be monitored using conventional techniques known in the art, such as by FACS analysis of cells expressing characteristic hematopoietic cell surface antigens in a blood sample withdrawn from the patient. For instance, a physician of skill in the art can withdraw a blood sample from the patient at various time points and determine the extent of donor CD134+ or CD278+ T cell reduction by conducting a FACS analysis to elucidate the relative concentrations of T cells in the sample using antibodies that bind to donor T cell antigens.

The anti-CD134 or anti-CD278 antibody, antigen-binding fragment thereof, drug-antibody conjugate, or drug-ligand conjugate can be administered to the patient in an aqueous solution containing one or more pharmaceutically acceptable excipients, such as a viscosity-modifying agent. The aqueous solution may be sterilized using techniques described herein or known in the art. The antibody, antigen-binding fragment thereof, drug-antibody conjugate, or drug-ligand conjugate can be administered to the patient at a dosage of, for example, from 0.001 mg/kg to 100 mg/kg prior to administration of a hematopoietic stem cell graft to the patient. The antibody, antigen-binding fragment thereof, drug-antibody conjugate, or drug-ligand conjugate can be administered to the patient at a time that optimally promotes engraftment of the exogenous hematopoietic stem cells, for instance, from about 1 hour to about 7 days (e.g., 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 2 days, 3 days, 4 days, 5 days, 6 days, or 7 days) or more prior to the administration of the exogenous hematopoietic stem cell transplant. For example, the antibody, antigen-binding fragment thereof, drug-antibody conjugate, or drug-ligand conjugate may be administered about 3 days prior to transplant. Alternatively, the antibody, antigen-binding fragment thereof, drug-antibody conjugate, or drug-ligand conjugate can be administered to the patient at a time that optimally promotes engraftment of the exogenous hematopoietic stem cells, for instance, concurrent with the administration of the exogenous hematopoietic stem cell transplant. Additionally, the antibody, antigen-binding fragment thereof, drug-antibody conjugate, or drug-ligand conjugate can be administered to the patient at a time that optimally promotes engraftment of the exogenous hematopoietic stem cells, for instance, from about 1 hour to about 10 days (e.g., 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 2 days, 3 days, 4 days, 5 days, 6 days, 7 days, 8 days, 9 days, or 10 days) or more after the administration of the exogenous hematopoietic stem cell transplant. For example, the antibody, antigen-binding fragment thereof, drug-antibody conjugate, or drug-ligand conjugate may be administered about 3 to 4 days after the transplant. The amount of antibody, antigen-binding fragment thereof, drug-antibody conjugate, or drug-ligand conjugate can be quantified, by methods known in the art, in the plasma of patients to determine when the concentration of antibody, antigen-binding fragment thereof, drug-antibody conjugate, or drug-ligand conjugate has reached its maximum.

The patient may then receive an infusion (e.g., an intravenous infusion) of exogenous hematopoietic stem cells, such as from the same physician that administered the antibody or antigen-binding fragment thereof or drug-antibody conjugate or from a different physician. The physician may administer the patient an infusion of autologous, syngeneic, or allogeneic hematopoietic stem cells, for instance, at a dosage of from 1×10³ to 1×10⁹ CD34⁺ cells/kg. The physician may monitor the engraftment of the hematopoietic stem cell transplant, for example, by withdrawing a blood sample from the patient and determining the increase in concentration of hematopoietic stem cells or cells of the hematopoietic lineage (such as megakaryocytes, thrombocytes, platelets, erythrocytes, mast cells, myeoblasts, basophils, neutrophils, eosinophils, microglia, granulocytes, monocytes, osteoclasts, antigen-presenting cells, macrophages, dendritic cells, natural killer cells, T cells, and B cells) following administration of the transplant. This analysis may be conducted, for example, from 1 hour to 6 months, or more, following hematopoietic stem cell transplant therapy (e.g., 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, or more). A finding that the concentration of hematopoietic stem cells or cells of the hematopoietic lineage has increased (e.g., by 1%, 2%, 3%, 4%, 5%, 6%, 7%, 8%, 9%, 10%, 20%, 30%, 40%, 50%, 60%, 70%, 80%, 90%, 100%, 200%, 500%, or more) following the transplant therapy relative to the concentration of the corresponding cell type prior to transplant therapy provides one indication that treatment with the anti-CD134 or anti-CD278 antibody, antigen-binding fragment thereof, drug-antibody conjugate, or drug-ligand conjugate has successfully promoted engraftment of the transplanted hematopoietic stem cell graft.

According to the methods disclosed herein, an anti-CD134 or anti-CD278 antibody or ADC may be administered to a human patient at risk for or suffering from GVHD. The antibody, fragment thereof, ADC, or soluble ligand may be covalently conjugated to a toxin, such as a cytotoxic molecule described herein or known in the art, or an Fc domain. For instance, an anti-CD134 or anti-CD278 antibody, antigen-binding fragment thereof, or soluble ligand can be covalently conjugated to a cytotoxin, such as a microtubule-binding agent, maytansine, a maytansinoid, an amatoxin, pseudomonas exotoxin A, deBouganin, diphtheria toxin, such as α-amanitin, saporin, an auristatin, an anthracycline, a calicheamicin, irinotecan, SN-38, a duocarmycin, a pyrrolobenzodiazepine, a pyrrolobenzodiazepine dimer, an indolinobenzodiazepine, and an indolinobenzodiazepine dimer, or a variant thereof.

This conjugation can be performed using covalent bond-forming techniques described herein or known in the art. The antibody, antigen-binding fragment thereof, or drug-antibody conjugate, or drug-ligand conjugate can subsequently be administered by intravenous administration, for example, to a patient at risk for GVHD. The antibody, antigen-binding fragment thereof, or drug-antibody conjugate, or drug-ligand conjugate can subsequently be administered by intravenous administration, for example, to a patient suffering from GVHD. For example, the antibody, antigen-binding fragment thereof, or drug-antibody conjugate, or drug-ligand conjugate can be administered to the patient prior, at the time of, or after the transplantation of exogenous hematopoietic stem cells (such as allogeneic hematopoietic stem cells).

The anti-CD134 or anti-CD278 antibody, antigen-binding fragment thereof, drug-antibody conjugate, or drug-ligand conjugate can be administered in an amount sufficient to reduce the quantity of host-reactive T cells, for example, by 10%, 20%, 30%, 40%, 50%, 60%, 70%, 80%, 90%, 95%, or more following hematopoietic stem cell transplant therapy. The reduction in donor T cell count can be monitored using conventional techniques known in the art, such as by FACS analysis of cells expressing characteristic hematopoietic cell surface antigens in a blood sample withdrawn from the patient. For instance, a physician of skill in the art can withdraw a blood sample from the patient at various time points and determine the extent of donor CD134+ or CD278+ T cell reduction by conducting a FACS analysis to elucidate the relative concentrations of T cells in the sample using antibodies that bind to donor T cell antigens.

The anti-CD134 or anti-CD278 antibody, antigen-binding fragment thereof, drug-antibody conjugate, or drug-ligand conjugate can be administered to the patient in an aqueous solution containing one or more pharmaceutically acceptable excipients, such as a viscosity-modifying agent. The aqueous solution may be sterilized using techniques described herein or known in the art. The antibody, antigen-binding fragment thereof, drug-antibody conjugate, or drug-ligand conjugate can be administered to the patient at a dosage of, for example, from 0.001 mg/kg to 100 mg/kg prior to administration of a hematopoietic stem cell graft to the patient. The antibody, antigen-binding fragment thereof, drug-antibody conjugate , or drug-ligand conjugate can be administered to the patient at a time that optimally promotes prevention and treatment of GVHD, for instance, from 1 hour to 7 days (e.g., 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 2 days, 3 days, 4 days, 5 days, 6 days, or 7 days) or more prior to the administration of the exogenous hematopoietic stem cell transplant. For example, the antibody, antigen-binding fragment thereof, drug-antibody conjugate, or drug-ligand conjugate may be administered about 3 days prior to transplant. Alternatively, the antibody, antigen-binding fragment thereof, drug-antibody conjugate, or drug-ligand conjugate can be administered to the patient at a time that optimally promotes engraftment of the exogenous hematopoietic stem cells, for instance, concurrent with the administration of the exogenous hematopoietic stem cell transplant. Additionally, the antibody, antigen-binding fragment thereof, drug-antibody conjugate, or drug-ligand conjugate can be administered to the patient at a time that optimally promotes engraftment of the exogenous hematopoietic stem cells, for instance, from about 1 hour to about 10 days (e.g., 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 2 days, 3 days, 4 days, 5 days, 6 days, 7 days, 8 days, 9 days, or 10 days) or more after the administration of the exogenous hematopoietic stem cell transplant . For example, the antibody, antigen-binding fragment thereof, drug-antibody conjugate, or drug-ligand conjugate may be administered about 3 to 4 days after the transplant. The amount of antibody, antigen-binding fragment thereof, drug-antibody conjugate, or drug-ligand conjugate can be quantified, by methods known in the art, in the plasma of patients to determine when the concentration of antibody, antigen-binding fragment thereof, drug-antibody conjugate, or drug-ligand conjugate has reached its maximum.

The patient may then receive an infusion (e.g., an intravenous infusion) of exogenous hematopoietic stem cells, such as from the same physician that administered the antibody or antigen-binding fragment thereof or drug-antibody conjugate, or from a different physician. The physician may administer the patient an infusion of autologous or allogeneic hematopoietic stem cells, for instance, at a dosage of from 1×10³ to 1×10⁹ CD34⁺ cells/kg.

A physician of skill in the art can evaluate the clinical manifestations of GVHD after administering to the human patient an antibody, antigen-binding fragment thereof, ADC, or soluble ligand capable of binding CD134 or CD278, such as an anti-CD134 or anti-CD278 antibody described herein.

According to the methods disclosed herein, an anti-CD134 or CD278 antibody or ADC may be administered to a human patient develops an autoimmune disease as a result of hematopoietic stem cell transplantation. According to the methods disclosed herein, a physician of skill in the art can administer to the human patient an antibody, antigen-binding fragment thereof, ADC, or soluble ligand capable of binding CD134 or CD278, such as an anti-CD134 or anti-CD278 antibody or ADC described herein. The antibody, fragment thereof, or soluble ligand may be covalently conjugated to a toxin, such as a cytotoxic molecule described herein or known in the art, or an Fc domain. For instance, an anti-CD134 or anti-CD278 antibody, antigen-binding fragment thereof, or soluble ligand can be covalently conjugated to a cytotoxin, such as a microtubule-binding agent, maytansine, a maytansinoid, an amatoxin, pseudomonas exotoxin A, deBouganin, diphtheria toxin, such as α-amanitin, saporin, an auristatin, an anthracycline, a calicheamicin, irinotecan, SN-38, a duocarmycin, a pyrrolobenzodiazepine, a pyrrolobenzodiazepine dimer, an indolinobenzodiazepine, and an indolinobenzodiazepine dimer, or a variant thereof.

This conjugation can be performed using covalent bond-forming techniques described herein or known in the art. The antibody, antigen-binding fragment thereof, or drug-antibody conjugate, or drug-ligand conjugate can subsequently be administered by intravenous administration, for example, to a patient at risk for an autoimmune disease (e.g., multiple sclerois, rheumatoid arthritis, intestinal bowel disease, psoriasis, lupus, and Type 1 diabetes). For example, the antibody, antigen-binding fragment thereof, or drug-antibody conjugate, or drug-ligand conjugate can be administered to the patient suffering from autoimmune disease that develops after the transplantation of exogenous hematopoietic stem cells (such as autologous or allogeneic hematopoietic stem cells).

The anti-CD134 or anti-CD278 antibody, antigen-binding fragment thereof, drug-antibody conjugate, or drug-ligand conjugate can be administered in an amount sufficient to reduce the quantity of host-reactive lymphocytes, for example, by 10%, 20%, 30%, 40%, 50%, 60%, 70%, 80%, 90%, 95%, or more prior to hematopoietic stem cell transplant therapy. The reduction in donor lymphocyte count can be monitored using conventional techniques known in the art, such as by FACS analysis of cells expressing characteristic hematopoietic cell surface antigens in a blood sample withdrawn from the patient. For instance, a physician of skill in the art can withdraw a blood sample from the patient at various time points and determine the extent of CD134+ or CD278+ T cell reduction by conducting a FACS analysis to elucidate the relative concentrations of T cells in the sample using antibodies that bind to T cell antigens. Efficacy against autoimmune disease can be measured by assays known in the art (e.g., autoantibody responses measurement from serum samples, and T cell proliferation in response to autoantigens).

The anti-CD134 or anti-CD278 antibody, antigen-binding fragment thereof, drug-antibody conjugate, or drug-ligand conjugate can be administered to the patient in an aqueous solution containing one or more pharmaceutically acceptable excipients, such as a viscosity-modifying agent. The aqueous solution may be sterilized using techniques described herein or known in the art. The antibody, antigen-binding fragment thereof, drug-antibody conjugate, or drug-ligand conjugate can be administered to the patient at a dosage of, for example, from 0.001 mg/kg to 100 mg/kg prior to administration of a hematopoietic stem cell graft to the patient. The antibody, antigen-binding fragment thereof, drug-antibody conjugate, or drug-ligand conjugate can be administered to the patient at a time that optimally promotes prevention and treatment of autoimmune disease, for instance, from about 1 hour to about 7 days (e.g., 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 2 days, 3 days, 4 days, 5 days, 6 days, or 7 days) or more prior to the administration of the exogenous hematopoietic stem cell transplant. For example, the antibody, antigen-binding fragment thereof, drug-antibody conjugate, or drug-ligand conjugate may be administered about 3 days prior to transplant. Alternatively, the antibody, antigen-binding fragment thereof, drug-antibody conjugate, or drug-ligand conjugate can be administered to the patient at a time that optimally promotes engraftment of the exogenous hematopoietic stem cells, for instance, concurrent with the administration of the exogenous hematopoietic stem cell transplant. Additionally, the antibody, antigen-binding fragment thereof, drug-antibody conjugate, or drug-ligand conjugate can be administered to the patient at a time that optimally promotes engraftment of the exogenous hematopoietic stem cells, for instance, from about 1 hour to about 10 days (e.g., 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 2 days, 3 days, 4 days, 5 days, 6 days, 7 days, 8 days, 9 days, or 10 days) or more after the administration of the exogenous hematopoietic stem cell transplant. For example, the antibody, antigen-binding fragment thereof, drug-antibody conjugate, or drug-ligand conjugate may be administered about 3 to 4 days after the transplant. The amount of antibody, antigen-binding fragment thereof, drug-antibody conjugate, or drug-ligand conjugate can be quantified, by methods known in the art, in the plasma of patients to determine when the concentration of antibody, antigen-binding fragment thereof, drug-antibody conjugate, or drug-ligand conjugate has reached its maximum.

The patient may then receive an infusion (e.g., an intravenous infusion) of exogenous hematopoietic stem cells, such as from the same physician that administered the antibody or antigen-binding fragment thereof or drug-antibody conjugate or from a different physician. The physician may administer the patient an infusion of autologous or allogeneic hematopoietic stem cells, for instance, at a dosage of from 1×10³ to 1×10⁹CD34⁺ cells/kg.

A physician of skill in the art can evaluate the clinical manifestations of autoimmune disease after administering to the human patient an antibody, antigen-binding fragment thereof, ADC, or soluble ligand capable of binding CD134 or CD278, such as an anti-CD134 or anti-CD278 antibody or ADC described herein.

According to the methods disclosed herein, an anti-CD134 or anti-CD278 antibody or ADC may be administered to a human patient at risk or suffering from an autoimmune disease. According to the methods disclosed herein, a physician of skill in the art can administer to the human patient an antibody, antigen-binding fragment thereof, ADC, or soluble ligand capable of binding CD134 or CD278, such as an anti-CD134 or anti-CD278 antibody or ADC described herein. The antibody, fragment thereof, or soluble ligand may be covalently conjugated to a toxin, such as a cytotoxic molecule described herein or known in the art, or an Fc domain.

This conjugation can be performed using covalent bond-forming techniques described herein or known in the art. The antibody, antigen-binding fragment thereof, or drug-antibody conjugate, or drug-ligand conjugate can subsequently be administered by intravenous administration, for example, to a patient at risk for autoimmune disease (e.g., multiple sclerois, rheumatoid arthritis, intestinal bowel disease, psoriasis, lupus, and Type 1 diabetes). The antibody, antigen-binding fragment thereof, or drug-antibody conjugate, or drug-ligand conjugate can subsequently be administered by intravenous administration, for example, to a patient suffering from autoimmune disease (e.g., multiple sclerois, rheumatoid arthritis, intestinal bowel disease, psoriasis, lupus, and Type 1 diabetes).

The anti-CD134 or anti-CD278 antibody, antigen-binding fragment thereof, drug-antibody conjugate, or drug-ligand conjugate can be administered in an amount sufficient to reduce the quantity of host-reactive lymphocytes, for example, by 10%, 20%, 30%, 40%, 50%, 60%, 70%, 80%, 90%, 95%, or more. The reduction in donor lymphocyte count can be monitored using conventional techniques known in the art, such as by FACS analysis of cells expressing characteristic hematopoietic cell surface antigens in a blood sample withdrawn from the patient. For instance, a physician of skill in the art can withdraw a blood sample from the patient at various time points and determine the extent of CD134+ or CD278+ T cell reduction by conducting a FACS analysis to elucidate the relative concentrations of T cells in the sample using antibodies that bind to T cell antigens. Efficacy against autoimmune disease can be measured by assays known in the art (e.g., autoantibody responses measurement from serum samples, and T cell proliferation in response to autoantigens).

The anti-CD134 or anti-CD278 antibody, antigen-binding fragment thereof, drug-antibody conjugate, or drug-ligand conjugate can be administered to the patient in an aqueous solution containing one or more pharmaceutically acceptable excipients, such as a viscosity-modifying agent. The aqueous solution may be sterilized using techniques described herein or known in the art. The antibody, antigen-binding fragment thereof, drug-antibody conjugate, or drug-ligand conjugate can be administered to the patient at a dosage of, for example, from 0.001 mg/kg to 100 mg/kg prior to administration of a hematopoietic stem cell graft to the patient. The antibody, antigen-binding fragment thereof, drug-antibody conjugate, or drug-ligand conjugate can be administered to the patient at a time that optimally promotes prevention and treatment of autoimmune disease. The amount of antibody, antigen-binding fragment thereof, drug-antibody conjugate, or drug-ligand conjugate can be quantified, by methods known in the art, in the plasma of patients to determine when the concentration of antibody, antigen-binding fragment thereof, drug-antibody conjugate, or drug-ligand conjugate has reached its maximum.

The methods herein provide a means by which alloreactive T cells, in particular, can be targeted specifically. Alloreactive T cells frequently arise in human patients following an allogeneic transplantation. One advantage of the therapies described herein is that the methods spare the patient's immune system by using specific targeting of either CD134 or CD278 cells such that the immune system is left largely intact allowing for greater immune recovery and protection from infections. Thus, in certain embodiments the methods are performed in the absence of conventional immune suppressing drugs, e.g., Methotrexate (Trexall®), cyclosporine, tacrolimus (Prograf®), mycophenolate mofetil (CellCept®), sirolimus (Rapamune®), corticosteroids (methylprednisolone or prednisone), antithymocyte globulin (ATG), alemtuzumab (Campath®) or cyclophosphamide (Cytoxan®).

The methods and compositions described herein can also, in certain embodiments, be used in combination with a conditioning therapy. As used herein, the terms “condition” and “conditioning” refer to processes by which a patient is prepared for receipt of a transplant containing stem cells, particularly hematopoietic stem cells. Such conditioning methods promote the engraftment of a hematopoietic stem cell transplant. A conditioning therapy involves administering to the transplant recipient (patient) an agent, such as an ADC, which selectively eliminates stem and/or immune cells from the patient prior to receiving the transplant, e.g., HSCs. For example, a patient may be conditioned for hematopoietic stem cell transplant therapy by administration to the patient of an antibody or antigen-binding fragment thereof capable of binding an antigen expressed by hematopoietic stem cells, such as CD117 or CD45. The antibody may be covalently conjugated to a cytotoxin so as to form an ADC. Administration of an antibody, antigen-binding fragment thereof, or drug-antibody conjugate capable of binding an antigen on a stem and/or immune cell to a patient in need of stem cell transplantation, including allogeneic stem cell transplantation, can promote the engraftment of a stem cell graft, for example, by selectively depleting endogenous hematopoietic stem cells, thereby creating a vacancy filled by an exogenous hematopoietic stem cell transplant. In one embodiment, the invention includes a method of conditioning a human patient for receiving a hematopoietic stem cell (HSC) transplantation, whereby an effective amount of an ADC comprising an anti-CD117 or an anti-CD45 monoclonal antibody and a cytotoxin (e.g., an amatoxin) is administered to the patient prior to transplantation, wherein the patient is also administered an anti-CD134 or an anti-CD278 ADC prior to, concomitantly with, and/or following the conditioning step in order to delete activated T cells expressing either CD134 or CD278. Such a combination therapy supports allogeneic transplantations by reducing the risk of or treating allograft rejection to the transplanted allogeneic cells. Examples of conditioning methods that may be used in combination with the methods and compositions disclosed herein can be found in U.S. Pat. No. 10,111,966; WO 2017/219025; and US 2016/0324982, each of which is incorporated by reference herein.

A physician of skill in the art can evaluate the clinical manifestations of autoimmune disease after administering to the human patient an antibody, antigen-binding fragment thereof, or soluble ligand capable of binding CD134 or CD278, such as an anti-CD134 or anti-CD278 antibody or ADC described herein.

### EXAMPLES

The following examples are put forth so as to provide those of ordinary skill in the art with a description of how the compositions and methods described herein may be used, made, and evaluated, and are intended to be purely exemplary of the invention and are not intended to limit the scope of what the inventors regard as their invention.

### Example 1

### Expression of CD134 on Resting and Activated T Cells

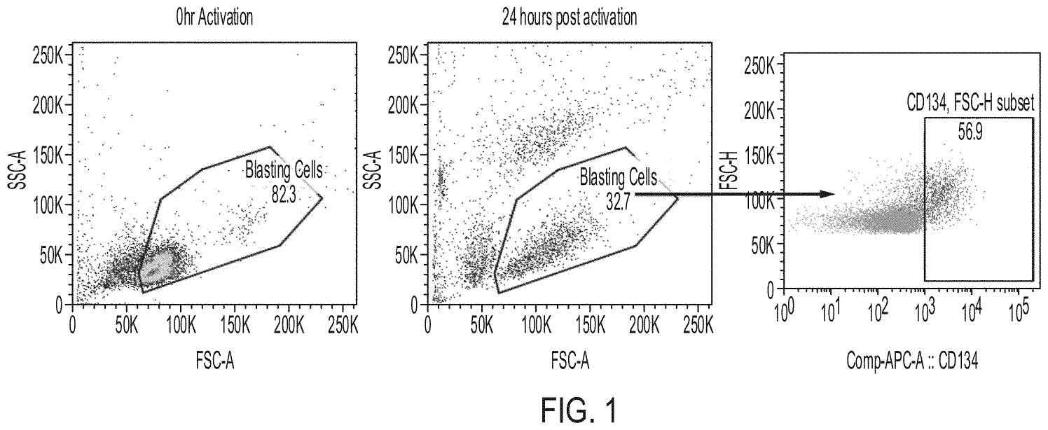

Expression of CD134 on both activated and resting T cells was determined. As shown in FIG. 1, at 24 hours past activation, T cells were 56.9% positive for CD134 (FIG. 1, third panel from left) compared to the stained 0 hour control (FIG. 1, first panel from left).

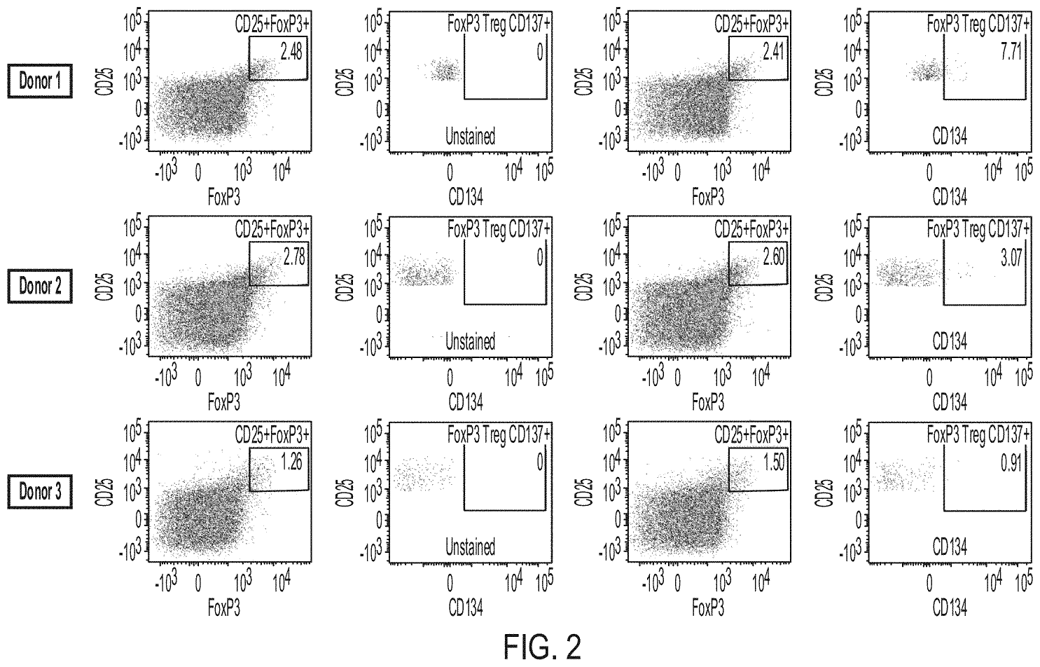

In FIG. 2, fresh whole blood from three individual healthy donor controls was evaluated for CD134 expression on regulatory T cells (Treg). Briefly, 50 μl of whole blood was added to a 96 well U bottom plate. Cells were stained with antibodies against human CD3, CD4, CD8, CD25, and CD134 for 30 minutes at 4° C. Red cells were lysed by adding 200 μl of lysis buffer (Qiagen) and incubating at room temperature for 10 minutes, twice. Cells were washed once in PBS and then fixed at room temperature in the dark for 45 minutes using Fix Solution (eBiosciences). Cells were washed twice in Perm Solution (eBiosciences) before being resuspended in 50 μl Perm Solution and stained with FoxP3 antibody or relevant isotype control. Stained samples were incubated at 4° C. for 30 minutes, washed with PBS, and run on a flow cytometer. Treg cells were determined as CD3+CD4+CD25+FoxP3+. CD134 levels were gated against a matched isotype control.

These data demonstrate that CD134 is expressed on activated T cells but is not significantly expressed on resting T cells or Tregs.

### Example 2

### T Cell Antibody Binding Assay

Anti-CD134 antibodies were tested to determine if they could bind to activated T cells.

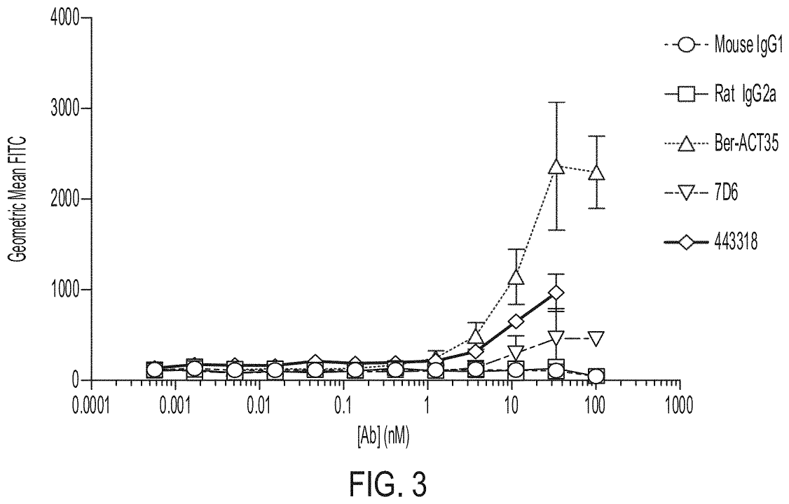

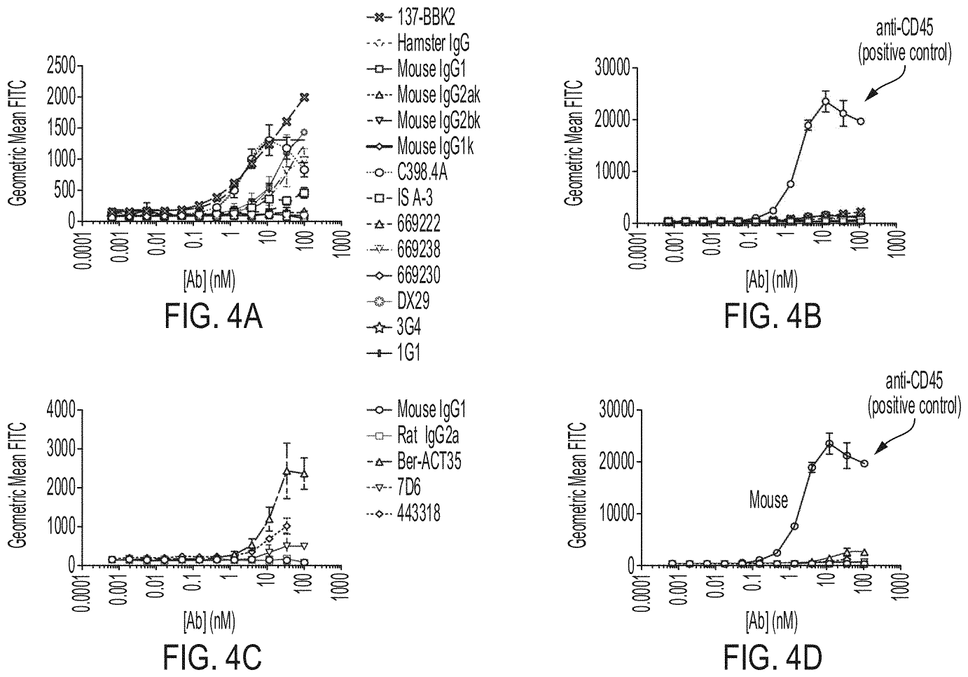

The T cell binding assays described in FIGS. 3 and 4 were performed according to the following protocol. Primary human CD3+ T cells were negatively selected from peripheral blood mononuclear cells. Cells were stimulated with anti-CD3/anti-CD28 beads (Invitrogen) overnight in AimV media at a bead:cell ratio of 0.5:1. The following day, 20,000 viable cells were plated per well and stained with a titration of primary antibody (anti-CD134 antibody or anti-CD278 antibody) for 4 hours at 4° C. Secondary anti-mouse AF488 stain at a constant amount was added for 30 minutes at 4° C. After washing, plates were run on a flow cytometer and binding was determined based on geometric mean fluorescence intensity in the AF488 channel.

As shown in FIG. 3, the anti-CD134 clone (antibody Ber-ACT35 (mouse IgG1)) showed binding to activated T cells. Antibody Ber-ACT35 is a murine anti-human CD134 antibody (Biolegend; Catalog #350002 (dated Jan. 17, 2019)). Anti-CD134 antibody 443318 (rat anti-human CD134 antibody (rat IgG2a); Novus; Catalog #MAB3388-SP (dated Jan. 17, 2019)) and anti-CD134 antibody 7D6 (mouse anti-human CD134 antibody (mouse IgG1); Thermo Fisher Scientific, Catalog #MA5-1648 (dated Jan. 17, 2019)) also showed binding to activated T cells above the negative control levels. A mouse IgG1 and a rat IgG2a were used as negative controls and did not show binding.

FIG. 4A describes binding of anti-CD278 antibodies to activated T cells. Antibody C398.4A (hamster anti-human CD278 antibody (BioLegend; Catalog #313502 (dated Jan. 17, 2019)), antibody ISA-3 (mouse anti-human CD278 antibody; Thermo Fisher; Catalog #14-9948-82(dated Jan. 17, 2019)), antibody 669222 (mouse anti-human CD278 antibody; Novus; Catalog #MAB6975 (dated Jan. 17, 2019)), antibody 669238 (mouse anti-human CD278 antibody; Novus; Catalog #MAB69752-SP (dated Jan. 17, 2019)), antibody 669230 (mouse anti-human CD278 antibody; Novus; Catalog #MAB69751-SP), anti-CD278 antibody DX29 (mouse anti-human CD278 antibody; BD Biosciences; Catalog #557801(dated Jan. 17, 2019)), antibody 3G4 (mouse anti-human Novus; Catalog #H00029851-M02 (dated Jan. 17, 2019)) and antibody 1G1 (mouse anti-human; Novus; Catalog #H00029851-M01 (dated Jan. 17, 2019)). Binding is observed with antibodies C398.4A, ISA-3, DX29, and 669238 above isotype control levels.

FIG. 4A also includes results for an anti-CD137 antibody (“137-BBK2”), which serves as a positive control because activated T cells are characterized by expression of CD137 on the cell surface. FIG. 4B shows the same data in FIG. 4A, but in comparison to results of a positive control, i.e., an anti-CD45 antibody. Activated T cells are characterized by high levels of expression of CD45 on the cell surface and thus, the data in FIG. 4B validated the binding assay used in FIG. 4A.

FIG. 4C describes binding of anti-CD134 antibodies to activated T cells (note these data are identical to the data in FIG. 3). FIG. 4D shows the same data in FIGS. 3 and 4A, but in comparison to the results from a positive control, i.e., an anti-CD45 antibody. Activated T cells are characterized by high levels of expression of CD45 on the cell surface and thus, the data in FIG. 4D validated the binding assay used in FIG. 4C.

### Example 3

### Anti-CD134 ADCs and Anti-CD278 ADCs Kill Primary Human T Cells

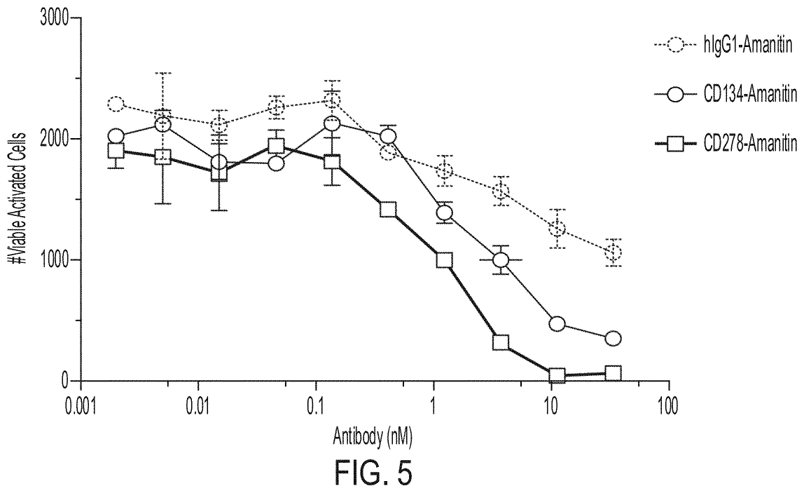

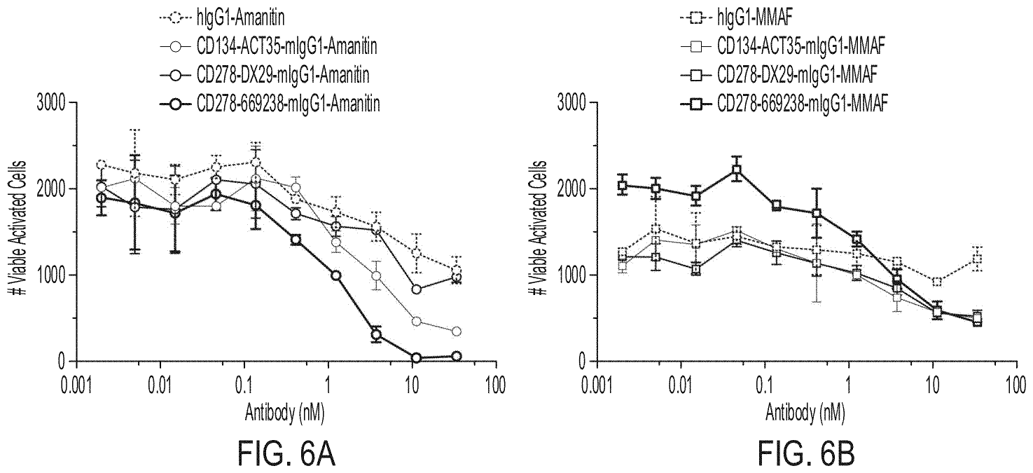

Primary human T cells were activated in the presence of ADCs targeting CD134 or CD278, and relevant controls. ADCs tested included a negative amanitin human IgG1 isotype control (i.e., M295), which is an antibody that is non-binding to either CD134 or CD278, and is conjugated to alpha amanitin via an MC cleavable linker (referred to as “hIgG1-Amanitin” in FIG. 5 and FIG. 6A), an anti-CD134-Amanitin ADC (i.e., M299), which is an antibody Ber-ACT35 (ACT35) conjugated to alpha amanitin via an MC cleavable linker (referred to as “CD134-Amanitin” in FIG. 5 and as “CD134-ACT35-mIgG1-Amanitin in FIG. 6A), an anti-CD278 ADC (i.e., M301), which is an antibody 669238 conjugated to alpha amanitin via an MC cleavable linker (referred to as “CD278-Amanitin” in FIG. 5 and as “CD278-669238-mIgG1-Amanitin” in FIG. 6A), and a further anti-CD278 ADC (i.e., M300), which is an antibody DX29 conjugated to alpha amanitin via an MC cleavable linker (referred to as “CD278-DX29-mIgG1-Amanitin” in FIG. 6A). Thus, the toxins and linkers were common among the ADCs tested in FIG. 5 and FIG. 6A.

The T cell killing assay in FIGS. 5 and 6 was performed as follows: cryopreserved negatively-selected primary human T cells were thawed and stimulated with anti-CD3/anti-CD28 beads (Invitrogen) at a bead:cell ratio of 0.5:1. At the start of the assay, 2×10⁴ T cells were seeded per well of a 384 well plate and antibodies were added to the cells at various concentrations between 30 nm and 0.003 nm before being placed in an incubator with 37° C. and 5% CO2. Following 4 days of culture, cells were analyzed by flow cytometry. Cells were stained with a viability marker Live/Dead Yellow (Invitrogen) and run on a volumetric flow cytometer. Numbers of viable, activated cells and viable, non-activated cells were determined by FSC vs SSC.

The results in FIG. 5 show that the number of viable, activated (blasting) T cells were dramatically decreased when exposed to either the anti-CD278-669238-Amanitin ADC (i.e., M301) or the anti-CD134-ACT35-Amanitin ADC (i.e., M299) compared to an isotype hIgG1-Amanitin ADC control (i.e., M295). Thus, both the CD134-Amanitin and the CD278-Amanitin ADCs showed killing in T cell assay with activated T cells. As described above, an hIgG1-Amanitin ADC (i.e., M295) was used as a negative control, and in addition, the CD134-amanitin ADC and the CD278-amanitin ADC contained an mIgG antibody. The ADC was also approximately 40% unconjugated. The results in FIG. 6A reproduce the same results described in FIG. 5, but also include results for the anti-CD278-DX29-Amanitin ADC (i.e., M300). These data demonstrated that the anti-CD278-DX29-Amanitin ADC (i.e., M300) is similarly capable of killing activated T cells.

The results shown in FIG. 6B show that the number of viable, activated (blasting) T cells were decreased upon exposure to the anti-CD134-ACT35-MMAF ADC (i.e., M307), the anti-CD278-DX29-MMAF ADC (i.e., M308), and the anti-CD278-669238-MMAF ADC (i.e., M309), compared to an isotype hIgG1-MMAF ADC control (i.e., M303). FIGS. 5 and 6 together demonstrate that the anti-CD134 and anti-CD278 antibodies conjugated to amanitin with the specific linker used here are more potent than those conjugated to MMAF with the specific linker used here.

### Example 4

### T Cell Killing Assay With Fab-Saporin

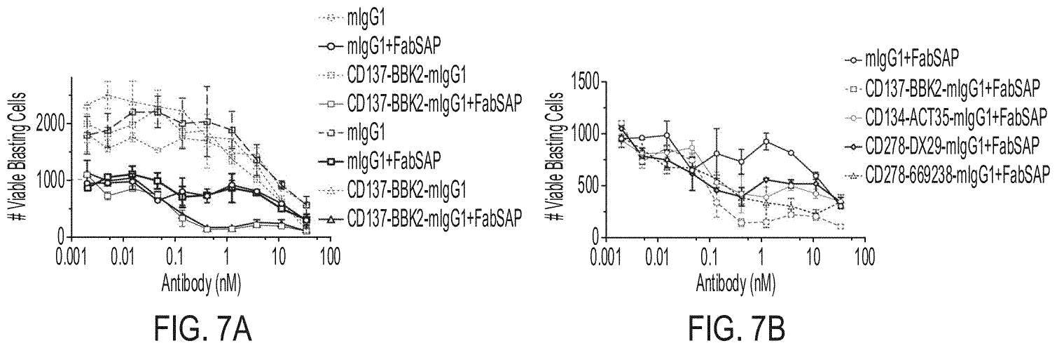

Anti-CD134 and anti-CD278 antibodies administered in combination with a Fab-Saporin were tested in a T cell killing assay, following the protocol described in Example 3. Results are provided in FIGS. 7A and 7B.

The results described in FIG. 7A include various positive (i.e., antibodies with Fab-SAP) and various negative controls (i.e., antibodies with Fab-SAP). These results indicate that the anti-CD137 antibody BBK2 (Thermo Fisher; catalog no. MA5-13739 and the mIgG1 isotype control were able to effectively kill T cells when administered in the presence of a Fab-Saporin. The results show that the cell numbers are lower in the Fab-Saporin groups regardless of antibody, as these Fab-Saporin have baseline toxicity to the cells (i.e, the isotype control and anti-CD137 antibody BBK2 have similar levels of cell loss at the lowest dilution). These results also indicate that the combination of anti-CD137 antibody BBK2 with Fab-Saporin was more effective at killing T cells than the combination of isotype mIgG1 with Fab-Saporin, likely due to the greater binding of the anti-CD137 antibody to activated T cells, which express CD137 on the cell surface. The results in FIG. 7B show that both the anti-CD134 antibody (ACT35) and the anti-CD278 antibodies (DX29 and 669238), when administered in combination with Fab-Saporin, are effective at killing activated T cells at levels similar to anti-CD137 antibody BBK2 with Fab-Saporin.

A key to the antibody descriptions is provided in the Table below:

| Sample | |
|---|---|
| ID/M# | mAb/L-T Description |
| M295 | hIgG isotype -MC-cleavable-α-Amanitin |
| M299 | Purified anti-human CD-134 (OX40) Be-ACT35 |
| | (ACT35)-MC-cleavable-α-Amanitin |
| M300 | Purified Mouse anti-Human CD278 |
| | (mIgG1 DX-29)-MC-cleavable-α-Amanitin |
| M301 | anti-ICOS (669238) MA69752-SP-MC-cleavable-α-Amanitin |
| M303 | hIgG isotype -MC-MMAF |
| M307 | Purified anti-human CD-134 (OX40) |
| | Be-ACT35 (ACT35)-MC-MMAF |
| M308 | Purified Mouse anti-Human CD278 |
| | (mIgG1 DX-29)-MC-MMAF |
| M309 | anti-ICOS (669238) MA69752-SP-MC-MMAF |

SEQUENCE TABLE

| Sequence | | |
|---|---|---|
| Identifier | Description | Sequence |
| SEQ ID NO: 1 | Human CD134 | MCVGARRLGRGPCAALLLLGLGLSTVTGLHCVGD |
| | (NCBI Reference | TYPSNDRCCHECRPGNGMVSRCSRSQNTVCRP |
| | Sequence: | CGPGFYNDWSSKPCKPCTWCNLRSGSERKQLC |
| | NP_003318.1) | TATQDTVCRCRAGTQPLDSYKPGVDCAPCPPGH |
| | | FSPGDNQACKPWTNCTLAGKHTLQPASNSSDAIC |
| | | EDRDPPATQPQETQGPPARPITVQPTEAWPRTS |
| | | QGPSTRPVEVPGGRAVAAILGLGLVLGLLGPLAIL |
| | | LALYLLRRDQRLPPDAHKPPGGGSFRTPIQEEQA |
| | | DAHSTLAKI |
| | Human CD278 | MKSGLWYFFLFCLRIKVLTGEINGSANYEMFIFHN |
| SEQ ID NO: 2 | (NCBI Reference | GGVQILCKYPDIVQQFKMQLLKGGQILCDLTKTKG |
| | Sequence. | SGNTVSIKSLKFCHSQLSNNSVSFFLYNLDHSHAN |
| | NP_036224.1) | YYFCNLSIFDPPPFKVTLTGGYLHIYESQLCCQLK |
| | | FWLPIGCAAFWVCILGCILICWLTKKKYSSSVHDP |
| | | NGEYMFMRAVNTAKKSRLTDVTL |

### Other Embodiments

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

1. A method of treating or preventing graft-versus-host disease (GVHD) in a human patient in need thereof, the method comprising administering to the patient an effective amount of an antibody, or antigen-binding fragment thereof, is capable of binding CD134, wherein the antibody or antigen-binding fragment thereof is conjugated to a cytotoxin via a linker.

2. A method of depleting a population of CD134 positive cells in a human patient suffering from or at risk for graft-versus-host disease (GVHD), the method comprising administering to the patient an effective amount of an antibody or antigen-binding fragment thereof capable of binding CD134, wherein the antibody or antigen-binding fragment thereof is conjugated to a cytotoxin via a linker.

3. A method of treating or preventing allograft rejection in a human patient in need thereof, the method comprising administering to the patient an effective amount of an antibody, or antigen-binding fragment thereof, is capable of binding CD134, wherein the antibody or antigen-binding fragment thereof is conjugated to a cytotoxin via a linker.

4. The method of claim 3, wherein the allograft rejection is host versus graft disease (HvGD).

5.-6. (canceled)

7. The method of claim 1, wherein the antibody or antigen-binding fragment thereof is a monoclonal antibody.

8. The method of claim 1, wherein the antibody has an isotype selected from the group consisting of IgG, IgA, IgM, IgD, and IgE.

9. The method of claim 8, wherein the antibody is an IgG and contains a human IgG1, IgG2, IgG3, or IgG4 isotype Fc domain.

10. The method of claim 1, wherein the cytotoxin is a microtubule-binding agent or an RNA polymerase inhibitor.

11.-12. (canceled)

13. The method of claim 10, wherein the RNA polymerase inhibitor is an amatoxin.

14. The method of claim 1, wherein the method comprises administering the antibody or antigen-binding fragment thereof to the human patient prior to the patient receiving a transplant comprising hematopoietic stem cells.

15. (canceled)

16. The method of claim 1, wherein the method comprises administering the antibody, antigen-binding fragment thereof, to the human patient concomitant with the patient receiving a transplant comprising hematopoietic stem cells.

17. The method of claim 1, wherein the method comprises administering the antibody, antigen-binding fragment thereof, to the human patient after the patient receives a transplant comprising hematopoietic stem cells.

18. The method of claim 17, the method comprising administering the antibody, antigen-binding fragment thereof, to the human patient about 1 hour to 10 days after the patient receives a transplant comprising hematopoietic stem cells.

19. (canceled)

20. The method of claim 14, wherein the transplant is a bone marrow transplant, a peripheral blood transplant, or a cord blood transplant.

21. The method of claim 1, wherein the human patient received an allogeneic transplant comprising hematopoietic stem cells.

22. The method of claim 2, wherein the CD134 positive cells are activated T cells.

23. The method of claim 1, wherein the antibody, antigen-binding fragment thereof, is internalized by a T cell upon contact.

24. The method of claim 1, wherein the antibody, antigen-binding fragment thereof promotes death or suppresses proliferation of a T cell.

25. The method of claim 1, wherein said patient is suffering from a stem cell disorder.

26. The method of claim 1, wherein said patient is suffering from a hemoglobinopathy disorder, an immunodeficiency disorder, a metabolic disorder, or cancer.

27.-59. (canceled)

60. An antibody drug conjugate (ADC) comprising an anti-CD134 antibody or an anti-CD278 antibody conjugated to a cytotoxin via a peptide linker, wherein the cytotoxin is a microtubule-binding agent or an RNA polymerase inhibitor.

61. The ADC of claim 60, wherein the RNA polymerase inhibitor is an amatoxin.

62. The anti-CD134 ADC of claim 61, wherein the amatoxin is an amanitin.

63. The anti-CD134 ADC of claim 62, wherein the amanitin is selected from the group consisting of α-amanitin, β-amanitin, γ-amanitin, ε-amanitin, amanin, amaninamide, amanullin, amanullinic acid, and proamanullin.

64. A pharmaceutical composition comprising the ADC of claim 60, and a pharmaceutically active carrier.

65.-136. (canceled)


SEQUENCE LISTING (2 sequences)

SEQ ID NO: 1 - protein, 277 aa, Homo sapiens
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 2 - protein, 199 aa, Homo sapiens
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL